(19) United States
(12) Patent Application Publication
(10) Pub. No.: US 2026/0190853 A1
(43) Pub. Date: Jul. 2, 2026

(54) ORGANIC ELECTROLUMINESCENT COMPOUND, COMPOSITION CONTAINING ORGANIC COMPOUND, AND ORGANIC ELECTROLUMINESCENT DEVICE

(71) Applicant: Shijiazhuang Chengzhi Yonghua Display Material Co., Ltd., Shijiazhuang, Hebei (CN)

(72) Inventors: Tianyu WEI, Shijiazhuang, Hebei (CN); Songyuan LIU, Shijiazhuang, Hebei (CN); Zhicheng CAO, Shijiazhuang, Hebei (CN); Zaifeng XIE, Shijiazhuang, Hebei (CN); Feng LIANG, Shijiazhuang, Hebei (CN)

(73) Assignee: Shijiazhuang Chengzhi Yonghua Display Material Co., Ltd., Shijiazhuang, Hebei (CN)

(21) Appl. No.: 18/292,974

(22) PCT Filed: Dec. 13, 2023
(86) PCT No.: PCT/CN2023/138463

§ 371 Date: Jan. 29, 2024

(30) Foreign Application Priority Data
Oct. 31, 2023 (CN) 202311436472.7

(51) Int. Cl. H10K 85/60; C07D 401/04; C09K 11/02; C09K 11/06; H10K 50/11

(52) U.S. Cl. CPC H10K 85/626; C07D 401/04; C09K 11/02; C09K 11/06; H10K 85/654; H10K 85/6572; H10K 85/6574; H10K 85/658; C09K 2211/1007; C09K 2211/1011; C09K 2211/1018; H10K 50/11

(57) ABSTRACT

The present invention provides an organic electroluminescent compound, a composition containing the organic compound, and an organic electroluminescent device. The organic electroluminescent compound has a general structural formula as shown in Formula I. The compound can not only enhance the electron-pushing ability and adjust the electron mobility of molecules, but also make the film formation of the material more stable and prolong the lifetime of the organic electroluminescent device. In addition, the present invention further provides a composition containing the organic electroluminescent compound. The composition can not only regulate the balance of carrier transport by means of interaction, but also reduce the energy loss. When the composition provided by the present invention is applied to a luminescent layer of a blue organic electroluminescent device, the luminescent layer material can improve the luminous efficiency of the blue organic electroluminescent device, prolong the lifetime of the blue organic electroluminescent device and overcome the defects in the prior art.

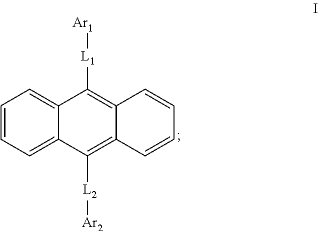

### TECHNICAL FIELD

The present invention belongs to the technical field of OLED and in particular relates to an organic electroluminescent compound, a composition containing the organic electroluminescent compound, and an organic electroluminescent device.

### BACKGROUND ART

Organic Light-Emitting Diode (OLED) is a display lighting technology which have been developed gradually in recent years. Especially in the display industry, OLED display has been widely concerned due to its advantages of high response, high contrast, flexibility, etc. At present, pixel units of full-color OLED display screens on the market are all composed of three primary colors, i.e., red, green and blue. According to the trichromatic theory, various colors can be generated by controlling the gray scales of the monochromatic colors red, green and blue of sub-pixel units, thus displaying color pictures. In three-color light-emitting devices, compared with red light and green light materials, a blue light material has a higher energy and can undergo energy transfer to low-energy organic luminescent materials of green light, yellow light, red light, etc., and according to the trichromatic theory, blue light emission is the basis of realizing white and colored display. Therefore, blue light materials are the focus of research in the field of organic photoelectric materials.

At present, in almost all luminescent layers in blue organic electroluminescent devices, host-guest doped luminescent systems are used, that is, electroluminescence is realized by doping a host material with a guest doping material. Generally, the luminous energy of the host material is necessarily larger than that of the guest doping material, and the luminescent layer where the host material is located is the main area for carrier recombination, and the carriers recombine in the luminescent layer to generate excitons. After absorbing the energy of the excitons, the host material transfers or transmits it to the guest doping material by means of Forster and Dexter energy transfers, so that the guest doping material is excited and emits light.

However, luminescent layers in existing blue organic electroluminescent devices are mainly made of a single host material, which cannot effectively regulate the transport of holes and electrons. Usually, the transport speed of holes is faster than that of electrons, which easily leads to imbalanced carrier recombination and in severe cases may affect the range of the exciton recombination region, thus reducing the luminous efficiency of the organic electroluminescent device. In addition, commonly used blue boron-nitrogen doping materials have planarity in their parent nucleus structures, and the doping material molecules tend to aggregate during film formation, causing concentration quenching, which seriously affects the efficiency and lifetime of organic electroluminescent devices. Therefore, it is urgent to develop a new luminescent layer material for organic electroluminescent devices.

### SUMMARY OF THE INVENTION

In view of this, the present invention provides an organic electroluminescent compound, a composition containing the organic electroluminescent compound, and an organic electroluminescent device. The organic electroluminescent compound can not only enhance the electron-pushing ability and adjust the electron mobility of molecules, but also make the film formation of the material more stable and prolong the lifetime of the organic electroluminescent device; in addition, the composition containing the organic electroluminescent compound can not only regulate the balance of carrier transport, but also reduce the energy loss, by means of interaction. When the composition provided by the present invention is applied to a luminescent layer of a blue organic electroluminescent device, the luminescent layer material can improve the luminous efficiency of the blue organic electroluminescent device, prolong the lifetime of the blue organic electroluminescent device and overcome the defects in the prior art.

In order to achieve the above object of the present invention, the present invention uses the following technical solution:

A first aspect of the present invention provides an organic electroluminescent compound, wherein the organic electroluminescent compound has a general structural formula as shown in Formula I:

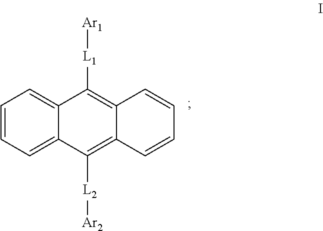

wherein Ar₁ is selected from a substituted or unsubstituted aryl group with a carbon atom number of 6 to 60 and a substituted or unsubstituted fused aryl group with a carbon atom number of 10 to 60;
L₁ and L₂ are each independently selected from a single bond and a substituted or unsubstituted arylene group with a carbon atom number of 6 to 30;
Ar₂ is

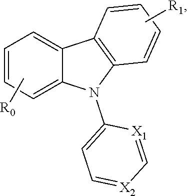

in which X₁ and X₂ are selected from C or N, and R₀ and R₁ respectively represent a single substituent to the maximum allowable number of substituents, and each of the substituents is independently selected from hydrogen, deuterium, a substituted or unsubstituted alkyl group with a carbon atom number of 1 to 10, and a substituted or unsubstituted alkenyl group with a carbon atom number of 2 to 10, wherein two or more of the substituents can be bonded to each other via a linker group or a single bond to form a benzene ring or a fused ring; and
when any one of Ar₁, L₁, L₂, R₀, and R₁ has substituents, Ar₁, L₁, L₂, R₀, and R₁ can have one or more substituents, and each of the substituents is independently selected from deuterium, an aryl group with a carbon atom number of 6 to 30, and a fused cyclic aryl group with a carbon atom number of 10 to 30.

The organic electroluminescent compound provided in the first aspect of the present invention contains an anthracene fragment and a pyridylcarbazole fragment, wherein the anthracene fragment has relatively strong conjugation and can transport both holes and electrons, and the carbazole group has a relatively strong electron-donating ability. In the present invention, introducing pyridyl into the carbazole and then connecting the pyridylcarbazole fragment to the anthracene fragment can not only enhance the electron-pushing ability of the compound and adjust the electron mobility of molecules, but also make the film formation of the material more stable and prolong the lifetime of the organic electroluminescent device.

In conjunction with the first aspect, Ar₂ is selected from any one of

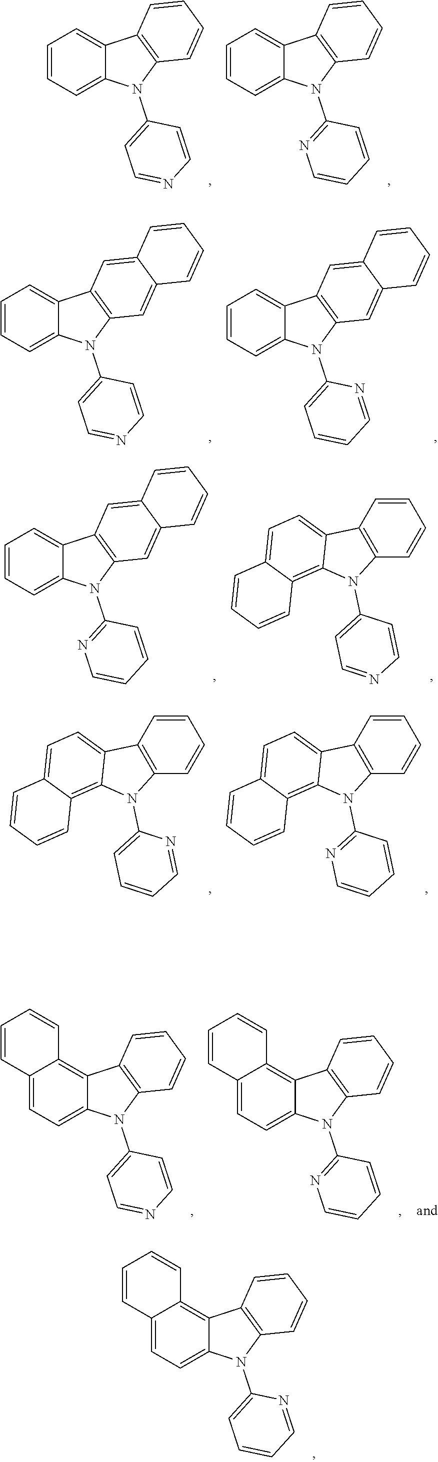

In conjunction with the first aspect, the organic electroluminescent compound represented is selected from any one of the following compounds:

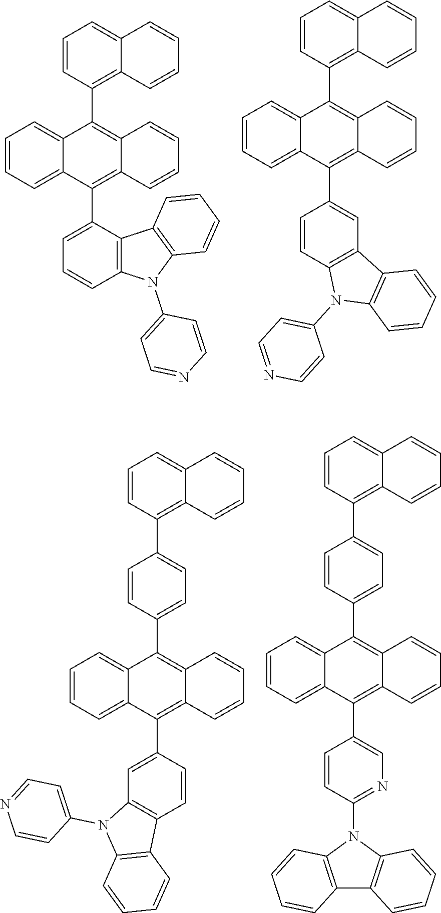

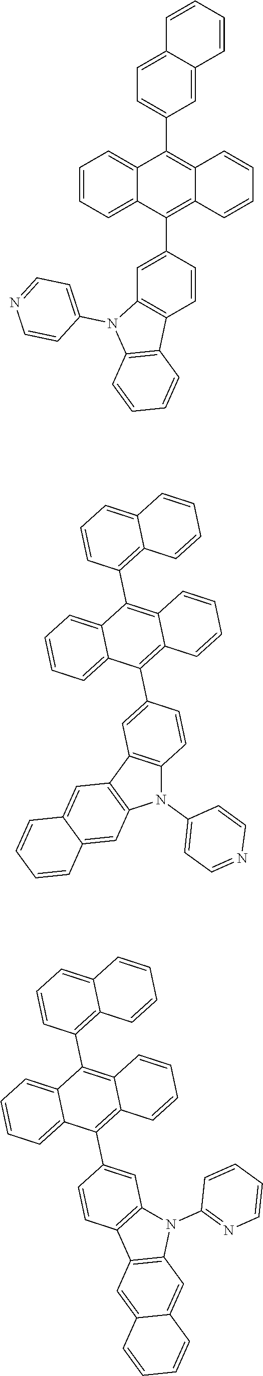 [SMILES: c1ccc2cc(-c3c4ccccc4c(-c4ccc5c6ccccc6n(-c6ccncc6)c5c4)c4ccccc34)ccc2c1 ; c1ccc2cc3c(cc2c1)c1cc(-c2c4ccccc4c(-c4cccc5ccccc45)c4ccccc24)ccc1n3-c1ccncc1 ; c1ccc(-n2c3cc(-c4c5ccccc5c(-c5cccc6ccccc56)c5ccccc45)ccc3c3cc4ccccc4cc32)nc1]

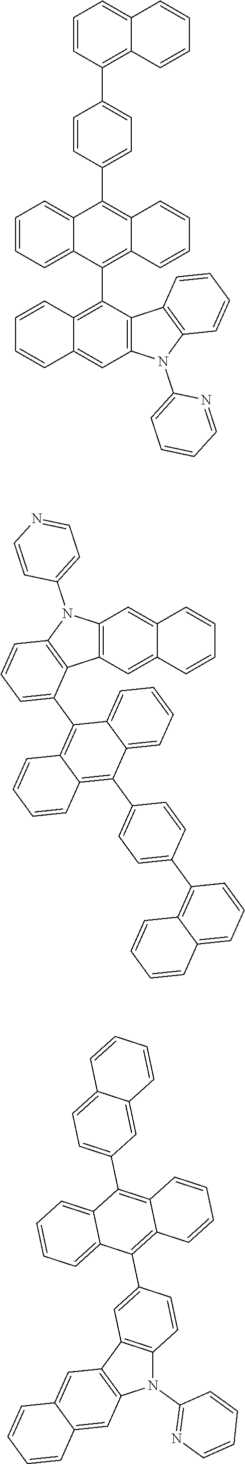 [SMILES: c1ccc(-n2c3ccccc3c3c(-c4c5ccccc5c(-c5ccc(-c6cccc7ccccc67)cc5)c5ccccc45)c4ccccc4cc32)nc1 ; c1ccc2cc3c(cc2c1)c1c(-c2c4ccccc4c(-c4ccc(-c5cccc6ccccc56)cc4)c4ccccc24)cccc1n3-c1ccncc1 ; c1ccc(-n2c3ccc(-c4c5ccccc5c(-c5ccc6ccccc6c5)c5ccccc45)cc3c3cc4ccccc4cc32)nc1]

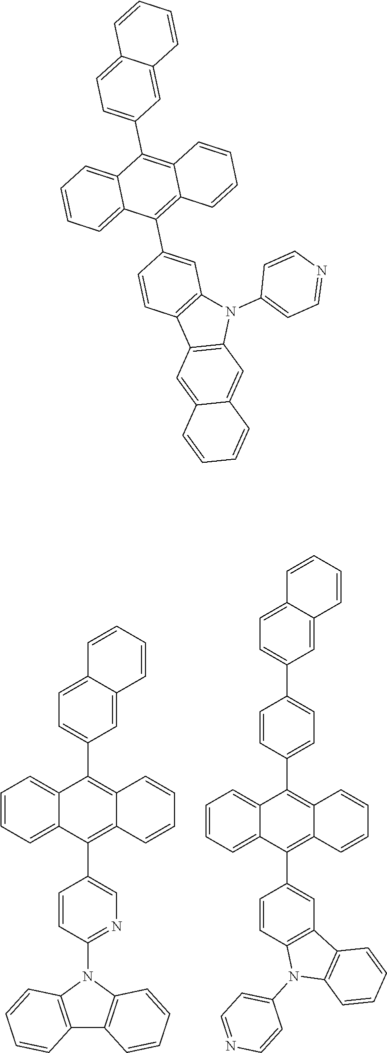

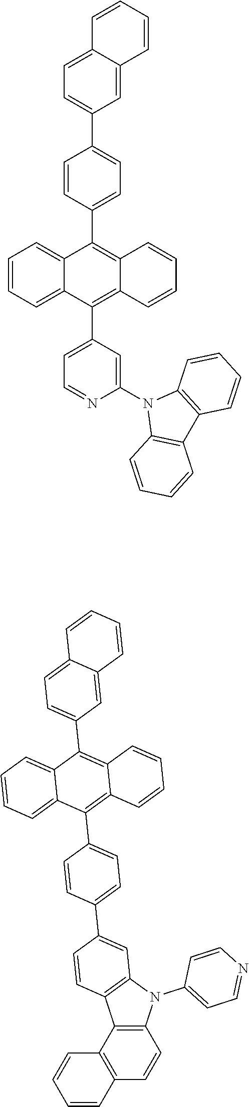 [SMILES: c1ccc2cc(-c3ccc(-c4c5ccccc5c(-c5ccnc(-n6c7ccccc7c7ccccc76)c5)c5ccccc45)cc3)ccc2c1 ; c1ccc2cc(-c3c4ccccc4c(-c4ccc(-c5ccc6c7c8ccccc8ccc7n(-c7ccncc7)c6c5)cc4)c4ccccc34)ccc2c1]

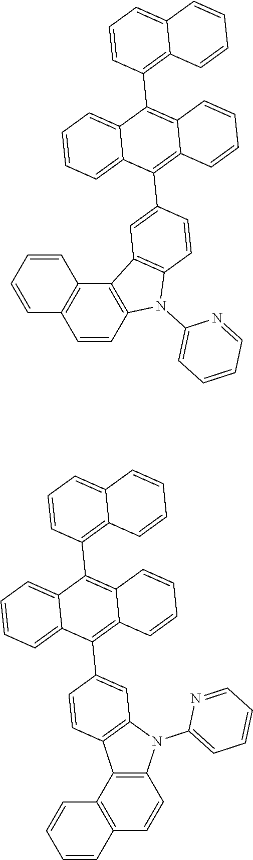 [SMILES: c1ccc(-n2c3ccc(-c4c5ccccc5c(-c5cccc6ccccc56)c5ccccc45)cc3c3c4ccccc4ccc32)nc1 ; c1ccc(-n2c3cc(-c4c5ccccc5c(-c5cccc6ccccc56)c5ccccc45)ccc3c3c4ccccc4ccc32)nc1]

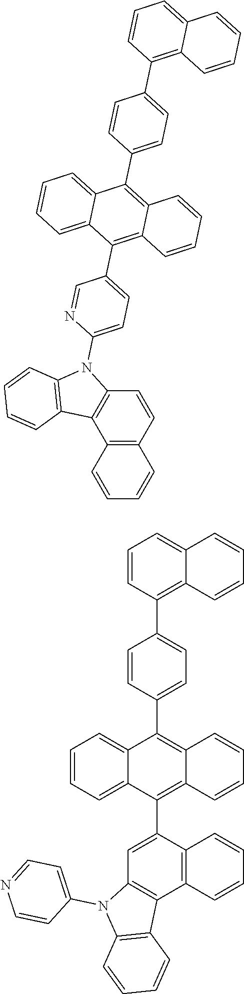

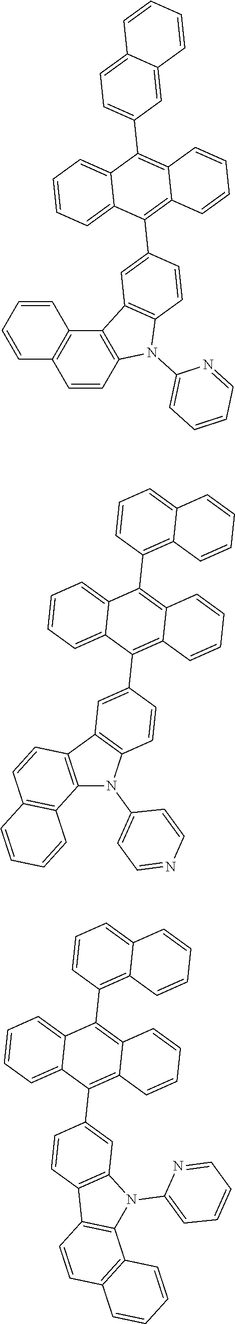 [SMILES: c1ccc(-n2c3ccc(-c4c5ccccc5c(-c5ccc6ccccc6c5)c5ccccc45)cc3c3c4ccccc4ccc32)nc1 ; c1ccc2c(-c3c4ccccc4c(-c4ccc5c(c4)c4ccc6ccccc6c4n5-c4ccncc4)c4ccccc34)cccc2c1 ; c1ccc(-n2c3cc(-c4c5ccccc5c(-c5cccc6ccccc56)c5ccccc45)ccc3c3ccc4ccccc4c32)nc1]

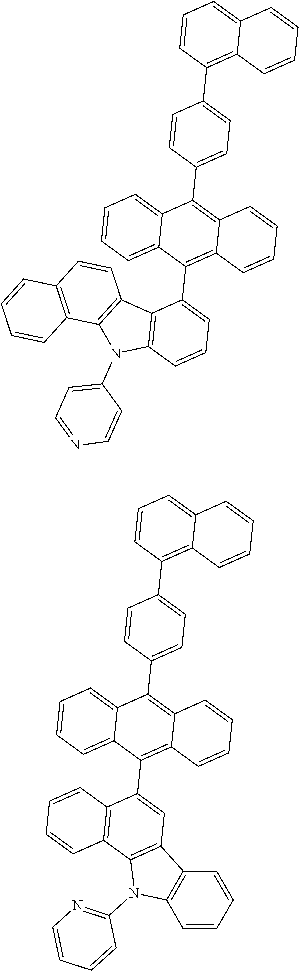 [SMILES: c1ccc2c(-c3ccc(-c4c5ccccc5c(-c5cccc6c5c5ccc7ccccc7c5n6-c5ccncc5)c5ccccc45)cc3)cccc2c1 ; c1ccc(-n2c3ccccc3c3cc(-c4c5ccccc5c(-c5ccc(-c6cccc7ccccc67)cc5)c5ccccc45)c4ccccc4c32)nc1]

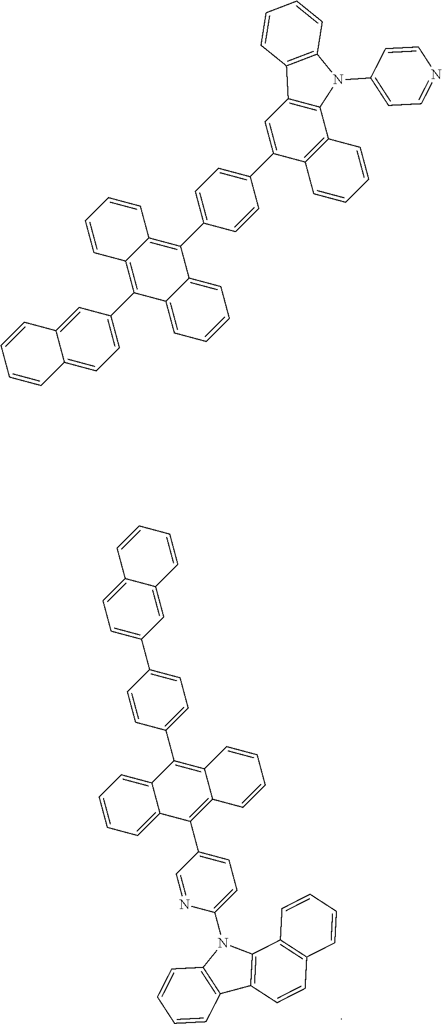 [SMILES: c1ccc2cc(-c3c4ccccc4c(-c4ccc(-c5cc6c7ccccc7n(-c7ccncc7)c6c6ccccc56)cc4)c4ccccc34)ccc2c1 ; c1ccc2cc(-c3ccc(-c4c5ccccc5c(-c5ccc(-n6c7ccccc7c7ccc8ccccc8c76)nc5)c5ccccc45)cc3)ccc2c1]

The composition provided according to the second aspect of the present invention comprises the compound represented by Formula I as described in the first aspect and further comprises a compound represented by Formula II containing a furan structure and a compound represented by Formula III containing a large steric hindrance and a thiophene structure:

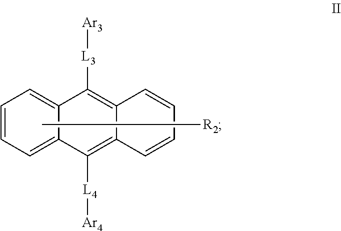

wherein Ar₃ is selected from a substituted or unsubstituted aryl group with a carbon atom number of 6 to 60 and a substituted or unsubstituted fused aryl group with a carbon atom number of 10 to 60;
R₂ represents a single substituent to the maximum allowable number of substituents, and each of the substituents is independently selected from hydrogen or deuterium;
L₃ and L₄ are each independently selected from a single bond and a substituted or unsubstituted arylene group with a carbon atom number of 6 to 30;
Ar₄ is

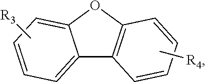

in which R₃ and R₄ represent a single substituent to the maximum allowable number of substituents, and each of the substituents is independently selected from hydrogen, deuterium, a substituted or unsubstituted alkyl group with a carbon atom number of 1 to 10, and a substituted or unsubstituted alkenyl group with a carbon atom number of 2 to 10, wherein two or more of the substituents can be bonded to each other via a linker group or a single bond to form a benzene ring or a fused ring; and
when any one of Ar₃, L₃, L₄, R₃, and R₄ has substituents, Ar₃, L₃, L₄, R₃, and R₄ can have one or more substituents, and each of the substituents is independently selected from deuterium, an aryl group with a carbon atom number of 6 to 30, and a fused cyclic aryl group with a carbon atom number of 10 to 30;

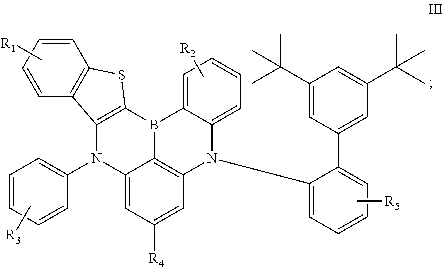

wherein R₅, R₆, and R₈ each independently represent a single substituent to the maximum allowable number of substituents, and each of the substituents is independently selected from any one of or a combination of any two or more of hydrogen, deuterium, a substituted or unsubstituted alkyl group with a carbon atom number of 1 to 10, a substituted or unsubstituted cycloalkyl group with a carbon atom number of 3 to 30, a substituted or unsubstituted aryl group with a carbon atom number of 6 to 30, and a substituted or unsubstituted arylamine group with a carbon atom number of 12 to 30, wherein two or more of the substituents can be bonded to each other via a linker group or a single bond to form an aliphatic ring, an aromatic ring, a heteroaromatic ring, a fused ring, or a fused heterocyclic ring;
R₇ represents a substituted or unsubstituted cycloalkyl group with a carbon atom number of 3 to 30 or a substituted or unsubstituted bridged cycloalkyl group with a carbon atom number of 4 to 30; and when R₇ has substituents, R₇ can have one or more substituents, each of which is independently selected from an alkyl group with a carbon atom number of 1 to 10 or an aryl group with a carbon atom number of 6 to 30, and two or more of the substituents can be bonded to each other via a linker group or a single bond to form an aliphatic ring;
R₉ represents a single substituent to the maximum allowable number of substituents, and each of the substituents is independently selected from any one of or a combination of any two or more of hydrogen, deuterium, a substituted or unsubstituted alkyl group with a carbon atom number of 1 to 10, and a substituted or unsubstituted aryl group with a carbon atom number of 6 to 30, wherein two or more of the substituents can be bonded to each other via a linker group or a single bond to form an aliphatic ring, an aromatic ring, a heteroaromatic ring, a fused ring, or a fused heterocyclic ring; and
when any one of R₅, R₆, R₈, and R₉ has substituents, R₅, R₆, R₈, and R₉ can have one or more substituents, and each of the substituents is independently selected from any one of deuterium, an alkyl group with a carbon atom number of 1 to 10, and a cycloalkyl group with a carbon atom number of 3 to 30.

The compound represented by Formula I provided by the present invention contains a pyridylcarbazole structure, which has electron-pushing property and can adjust the electron cloud density on the anthracene ring so as to facilitate hole transport. The compound represented by Formula II provided by the present invention contains a furan structure, which has electron-pulling property and can adjust the electron cloud density on the anthracene ring so as to facilitate electron transport. Compared with fused cyclic anthracene-based compounds, the mixture formed by the interaction between the compound represented by Formula I and the compound represented by Formula II in the present invention is more suitable for the transport of electrons and holes and can significantly balance the carrier transport. In the composition provided by the present invention, by separately defining the structures of the compounds represented by Formulas I, II and III and by means of the interaction between the compound represented by Formula III and the compounds represented by Formulas I and II, the molecules of the compound represented by Formula III in the composition can be horizontally distributed in the mixture of the compounds represented by Formulas I and II, such that the light emitted from the compound represented by Formula III tends to be emitted in the direction perpendicular to the substrate, thereby reducing the energy loss caused during light transmission.

In conjunction with the second aspect, in the composition, the mass ratio of the compound represented by Formula I to the compound represented by Formula II to the compound represented by Formula III is 49:49:2.

In conjunction with the second aspect, in the composition, Ar₄ is selected from any one of

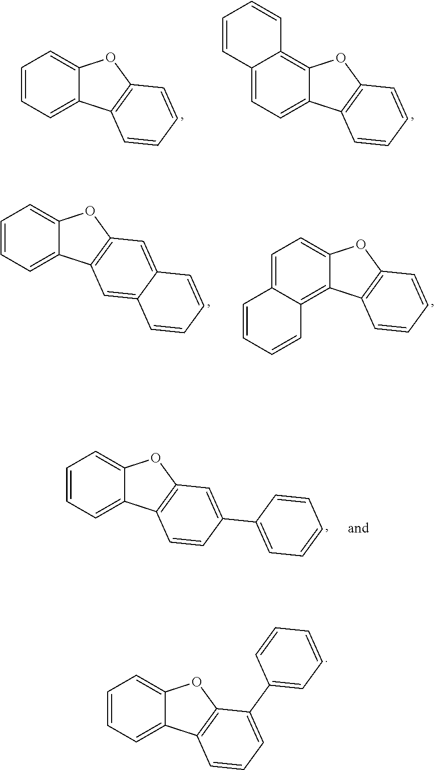

In conjunction with the second aspect, the compound represented by Formula II is selected from any one of the following compounds:

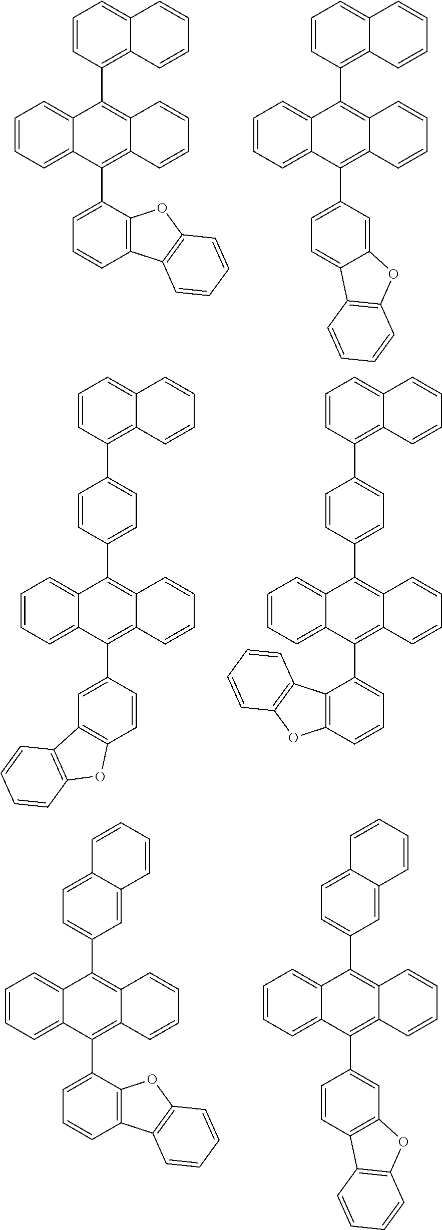

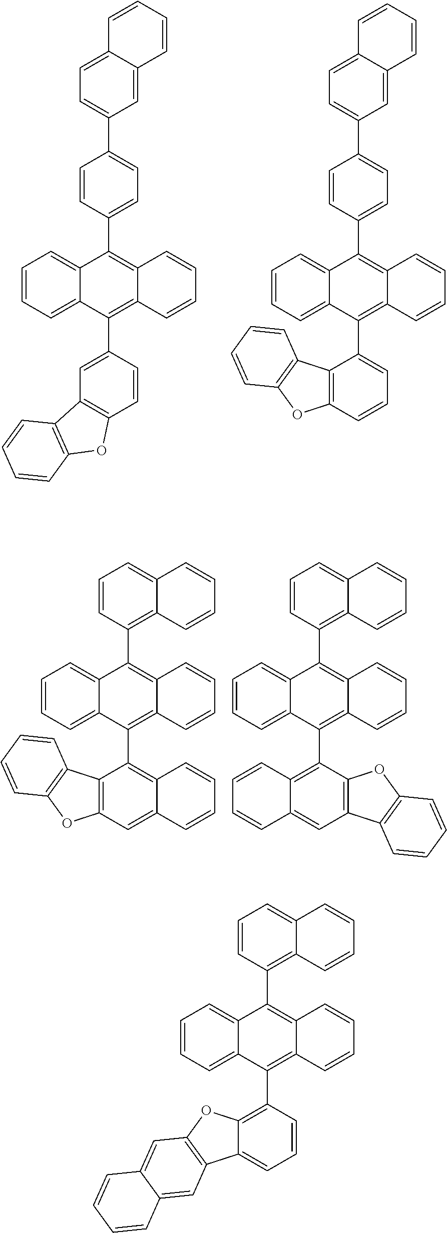

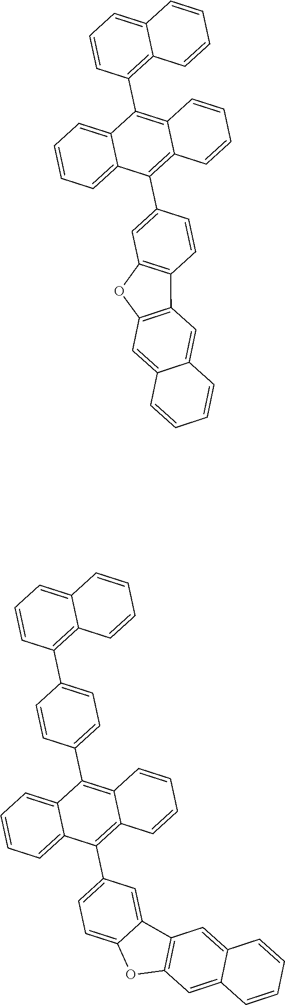 [SMILES: c1ccc2cc3c(cc2c1)oc1cc(-c2c4ccccc4c(-c4cccc5ccccc45)c4ccccc24)ccc13 ; c1ccc2cc3c(cc2c1)oc1ccc(-c2c4ccccc4c(-c4ccc(-c5cccc6ccccc56)cc4)c4ccccc24)cc13]

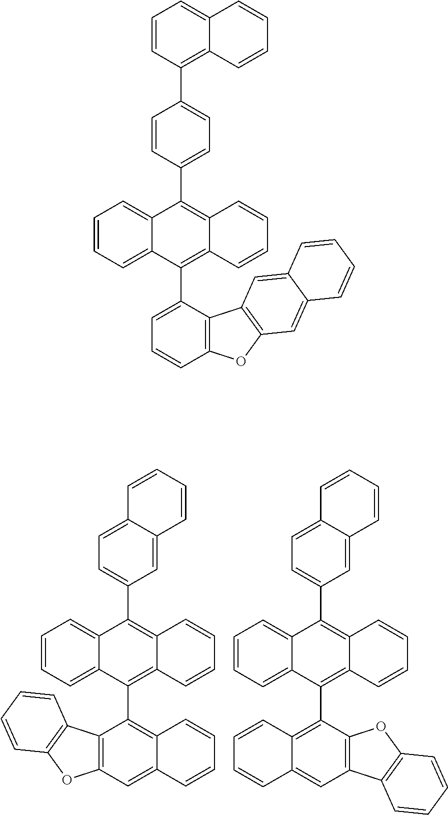

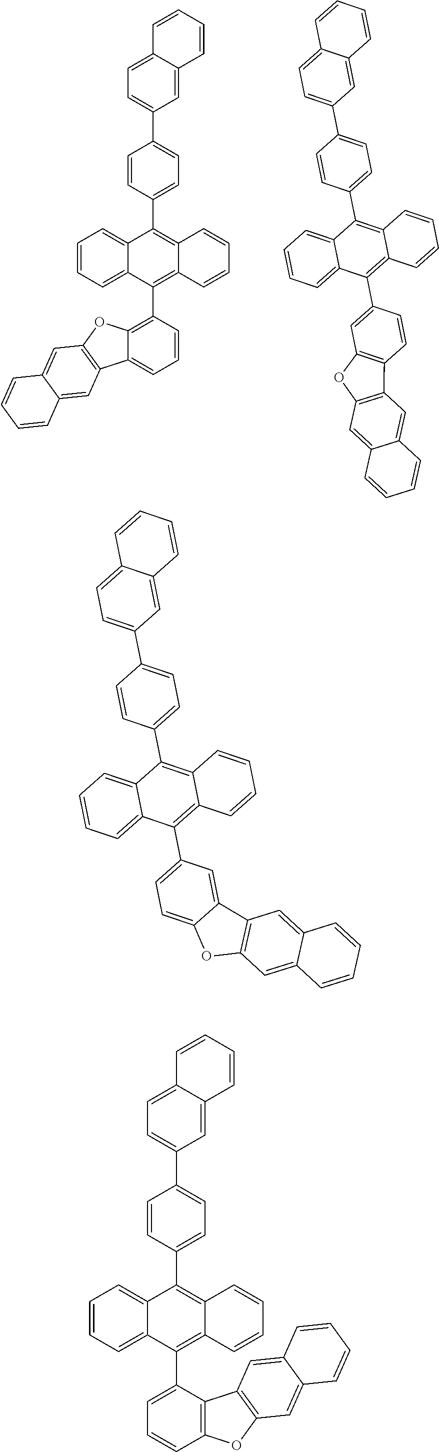 [SMILES: c1ccc2cc(-c3ccc(-c4c5ccccc5c(-c5cccc6c5oc5cc7ccccc7cc56)c5ccccc45)cc3)ccc2c1 ; c1ccc2cc(-c3ccc(-c4c5ccccc5c(-c5ccc6c(c5)oc5cc7ccccc7cc56)c5ccccc45)cc3)ccc2c1 ; c1ccc2cc(-c3ccc(-c4c5ccccc5c(-c5ccc6oc7cc8ccccc8cc7c6c5)c5ccccc45)cc3)ccc2c1 ; c1ccc2cc(-c3ccc(-c4c5ccccc5c(-c5cccc6oc7cc8ccccc8cc7c56)c5ccccc45)cc3)ccc2c1]

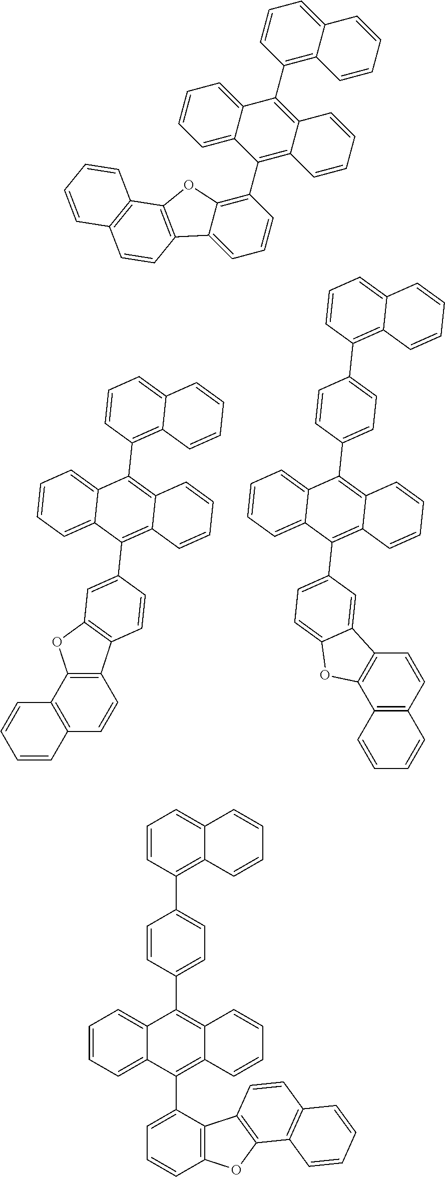

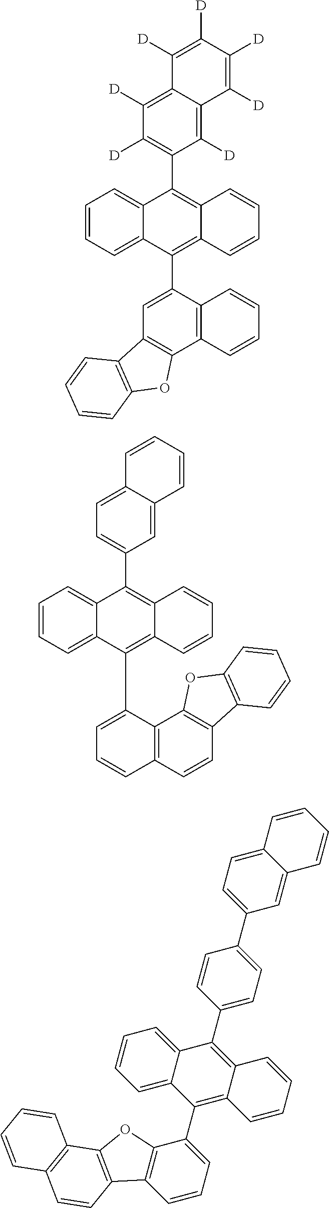

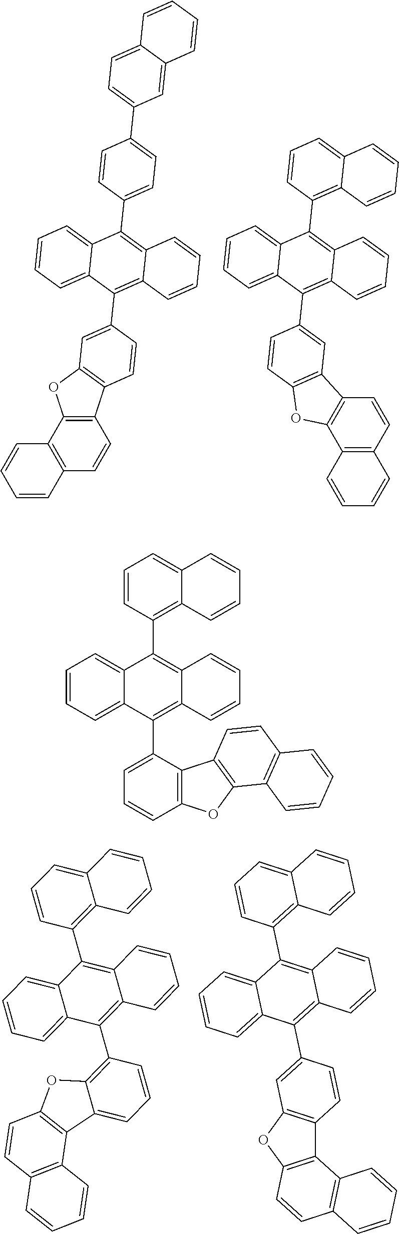

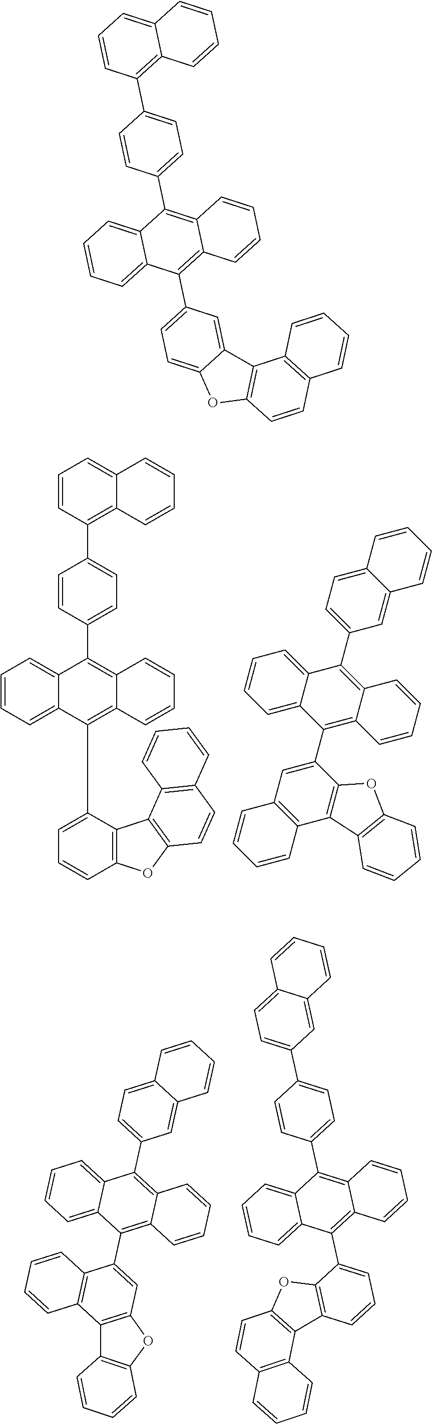

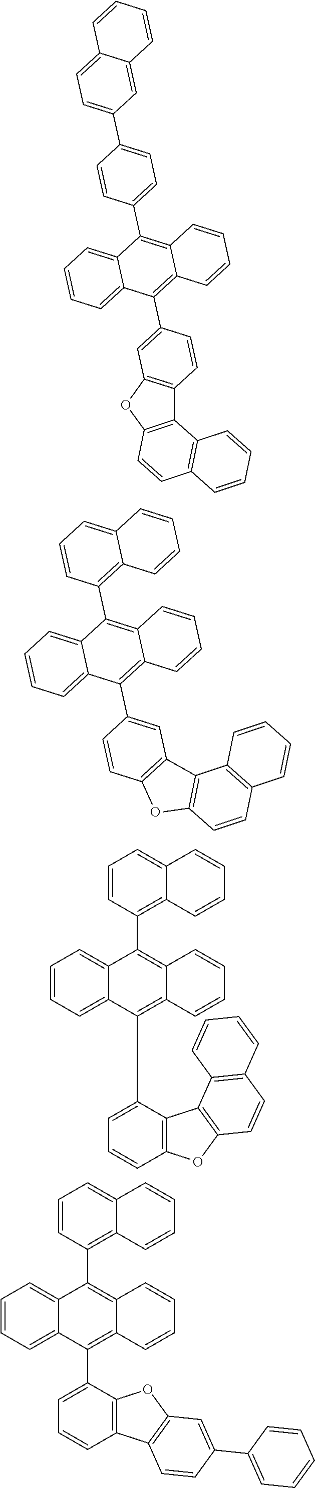

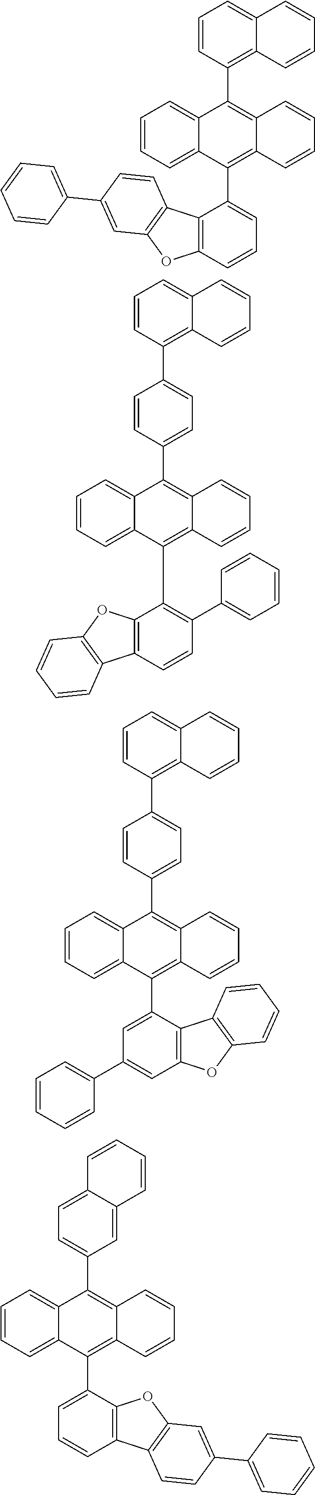

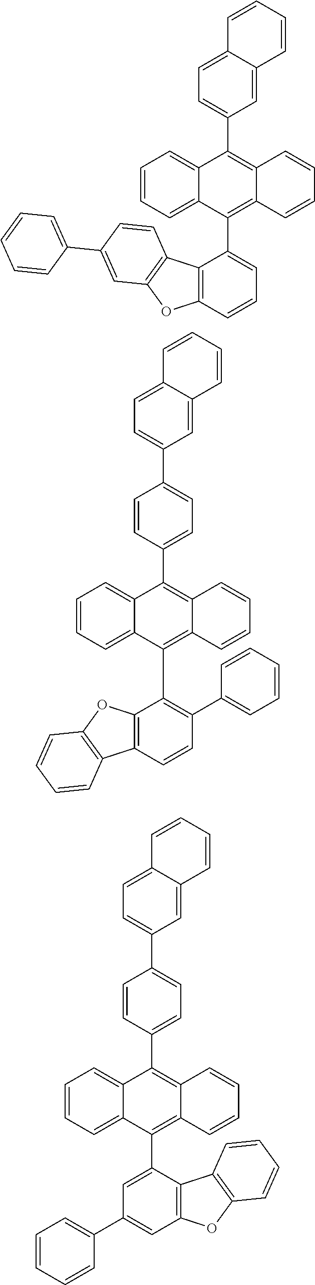

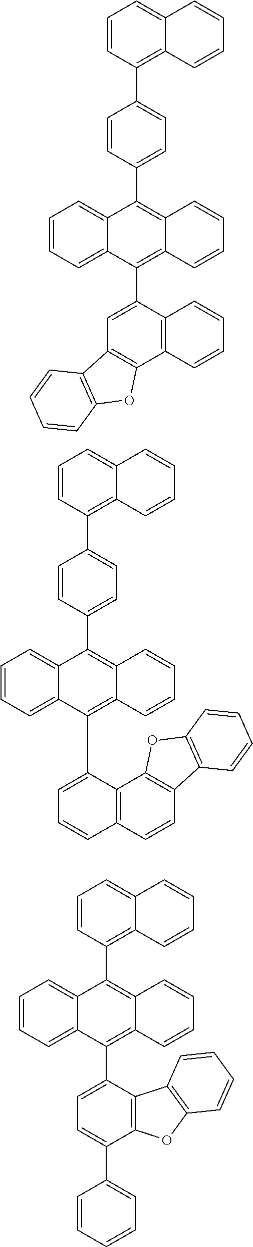

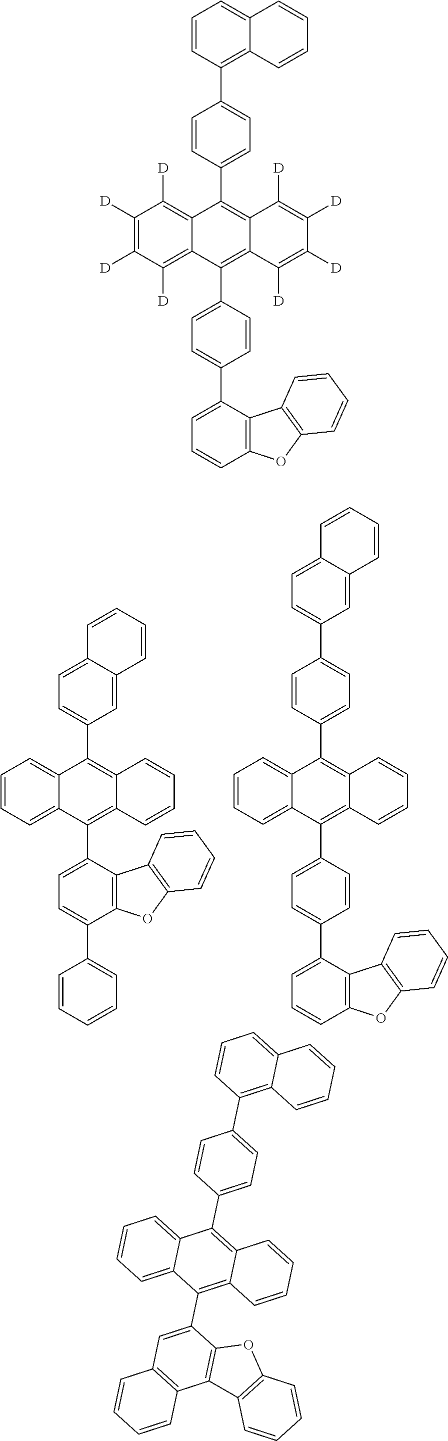

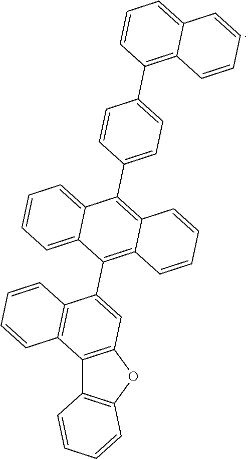 [SMILES: c1ccc2c(-c3ccc(-c4c5ccccc5c(-c5cc6oc7ccccc7c6c6ccccc56)c5ccccc45)cc3)cccc2c1]

In conjunction with the second aspect, the compound represented by Formula III is selected from any one of the following structures represented by Formulas III-1 to III-2:

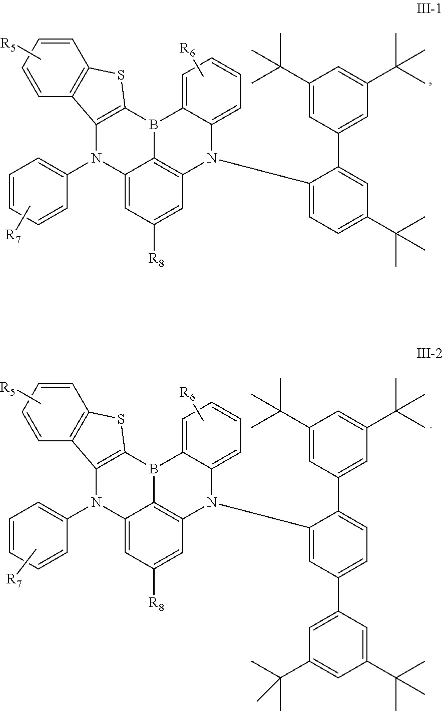

In conjunction with the second aspect, the compound represented by Formula III is selected from any one of the following compounds:

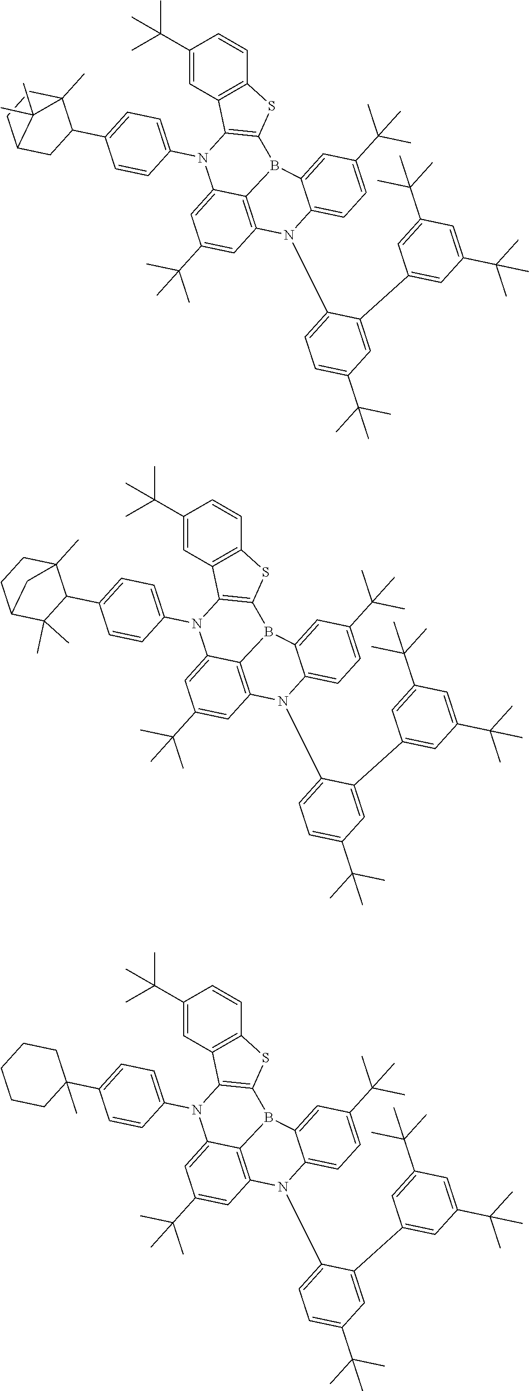 [SMILES: CC(C)(C)c1cc(-c2cc(C(C)(C)C)ccc2N2c3ccc(C(C)(C)C)cc3B3c4sc5ccc(C(C)(C)C)cc5c4N(c4ccc(C5CC6CCC5(C)C6(C)C)cc4)c4cc(C(C)(C)C)cc2c43)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(C5C6(C)CCC(C6)C5(C)C)cc4)c4cc(C(C)(C)C)cc2c43)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(C5(C)CCCCC5)cc4)c4cc(C(C)(C)C)cc2c43)cc(C(C)(C)C)c1]

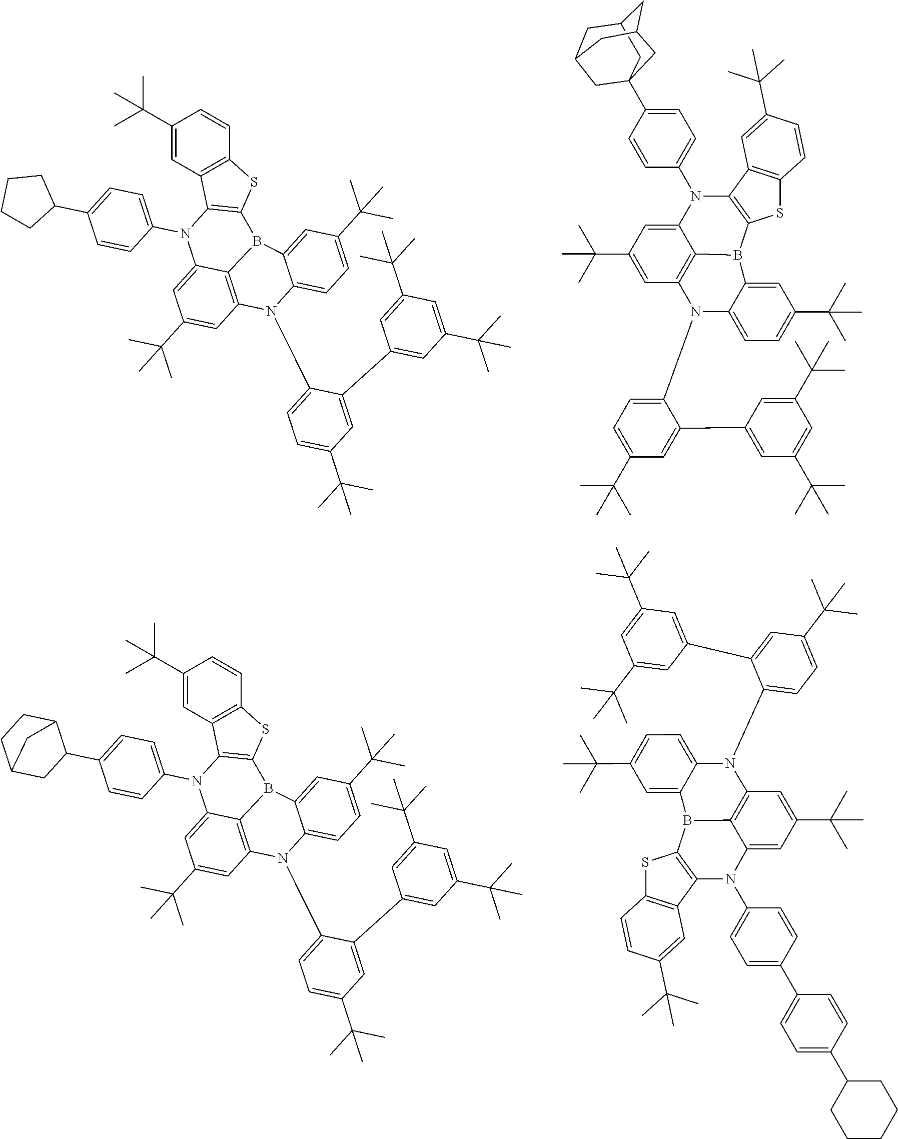

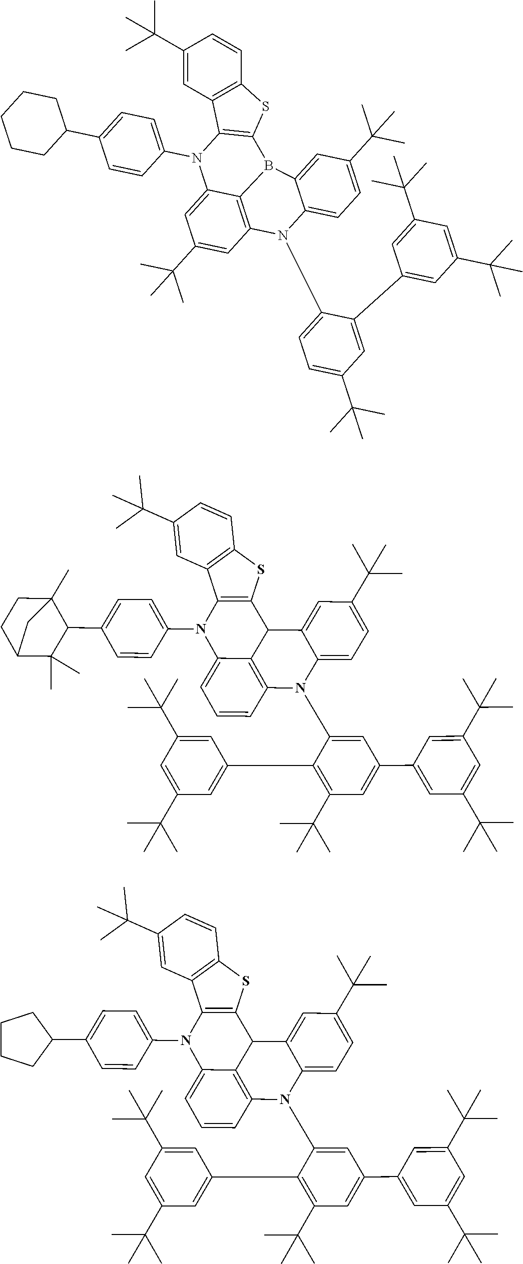

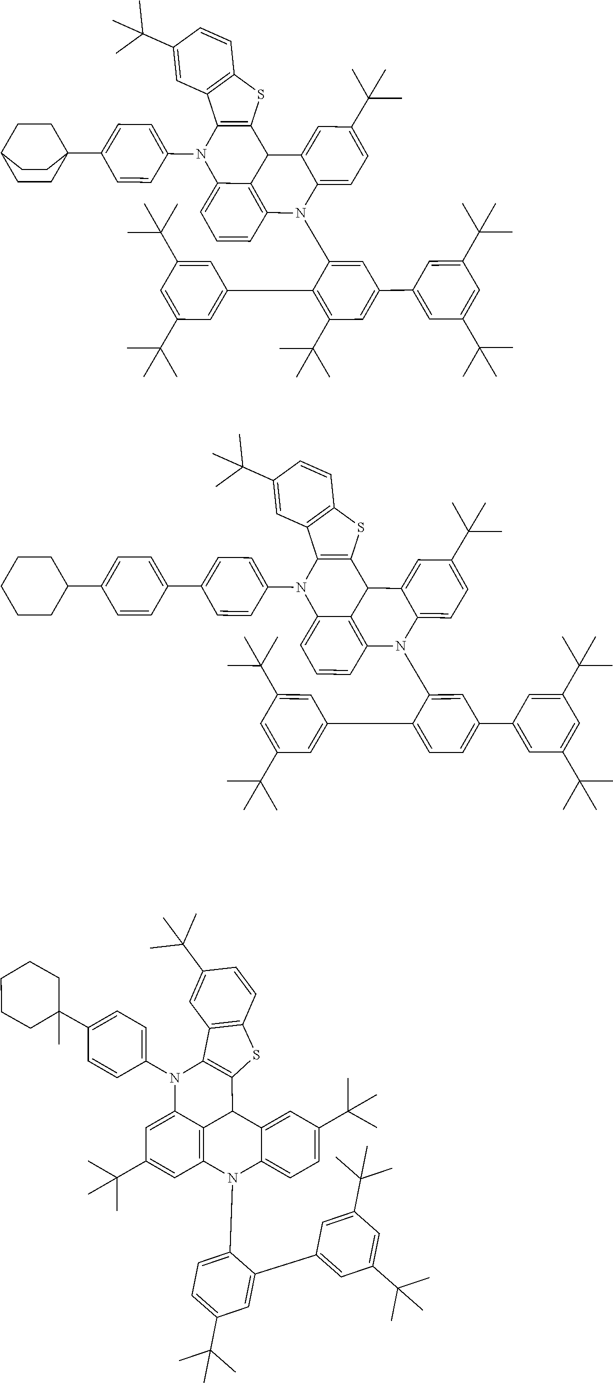 [SMILES: CC(C)(C)c1cc(-c2cc(N3c4ccc(C(C)(C)C)cc4C4c5sc6ccc(C(C)(C)C)cc6c5N(c5ccc(C67CCC(CC6)CC7)cc5)c5cccc3c54)c(-c3cc(C(C)(C)C)cc(C(C)(C)C)c3)c(C(C)(C)C)c2)cc(C(C)(C)C)c1 ; CC(C)(C)c1cc(-c2ccc(-c3cc(C(C)(C)C)cc(C(C)(C)C)c3)c(N3c4ccc(C(C)(C)C)cc4C4c5sc6ccc(C(C)(C)C)cc6c5N(c5ccc(-c6ccc(C7CCCCC7)cc6)cc5)c5cccc3c54)c2)cc(C(C)(C)C)c1 ; CC(C)(C)c1cc(-c2cc(C(C)(C)C)ccc2N2c3ccc(C(C)(C)C)cc3C3c4sc5ccc(C(C)(C)C)cc5c4N(c4ccc(C5(C)CCCCC5)cc4)c4cc(C(C)(C)C)cc2c43)cc(C(C)(C)C)c1]

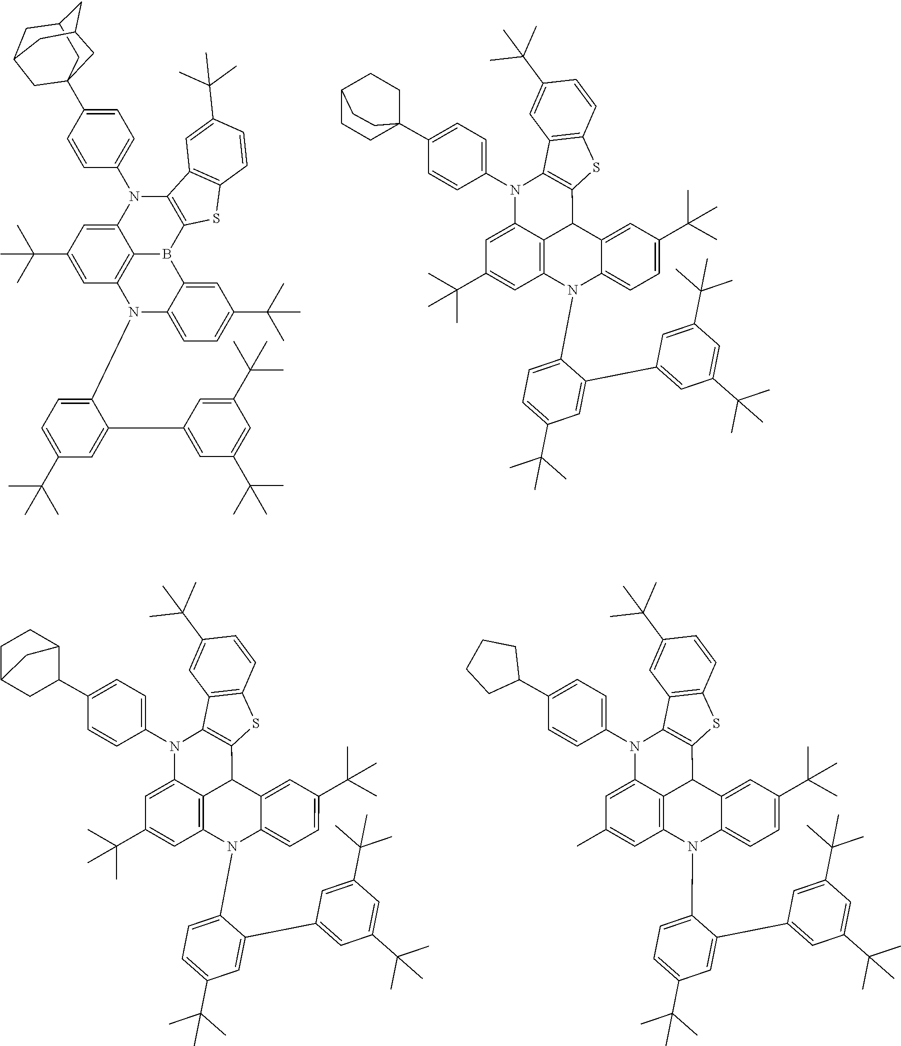

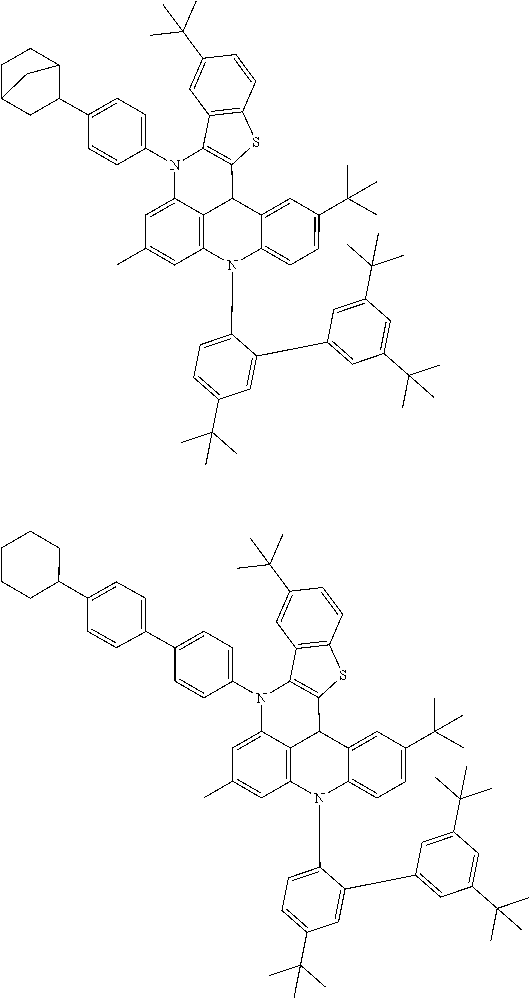 [SMILES: Cc1cc2c3c(c1)N(c1ccc(C4CC5CCC4C5)cc1)c1c(sc4ccc(C(C)(C)C)cc14)C3c1cc(C(C)(C)C)ccc1N2c1ccc(C(C)(C)C)cc1-c1cc(C(C)(C)C)cc(C(C)(C)C)c1 ; Cc1cc2c3c(c1)N(c1ccc(-c4ccc(C5CCCCC5)cc4)cc1)c1c(sc4ccc(C(C)(C)C)cc14)C3c1cc(C(C)(C)C)ccc1N2c1ccc(C(C)(C)C)cc1-c1cc(C(C)(C)C)cc(C(C)(C)C)c1]

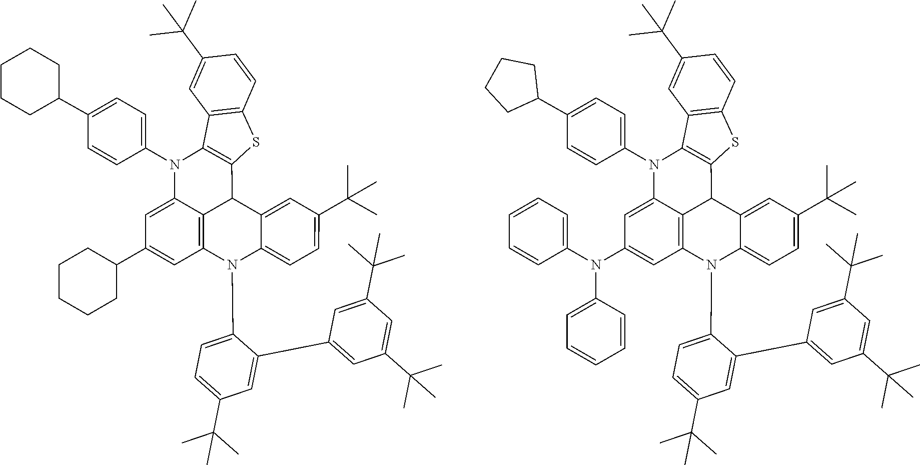 [SMILES: CC(C)(C)c1cc(-c2cc(C(C)(C)C)ccc2N2c3ccc(C(C)(C)C)cc3C3c4sc5ccc(C(C)(C)C)cc5c4N(c4ccc(C5CCCCC5)cc4)c4cc(C5CCCCC5)cc2c43)cc(C(C)(C)C)c1 ; CC(C)(C)c1cc(-c2cc(C(C)(C)C)ccc2N2c3ccc(C(C)(C)C)cc3C3c4sc5ccc(C(C)(C)C)cc5c4N(c4ccc(C5CCCC5)cc4)c4cc(N(c5ccccc5)c5ccccc5)cc2c43)cc(C(C)(C)C)c1]

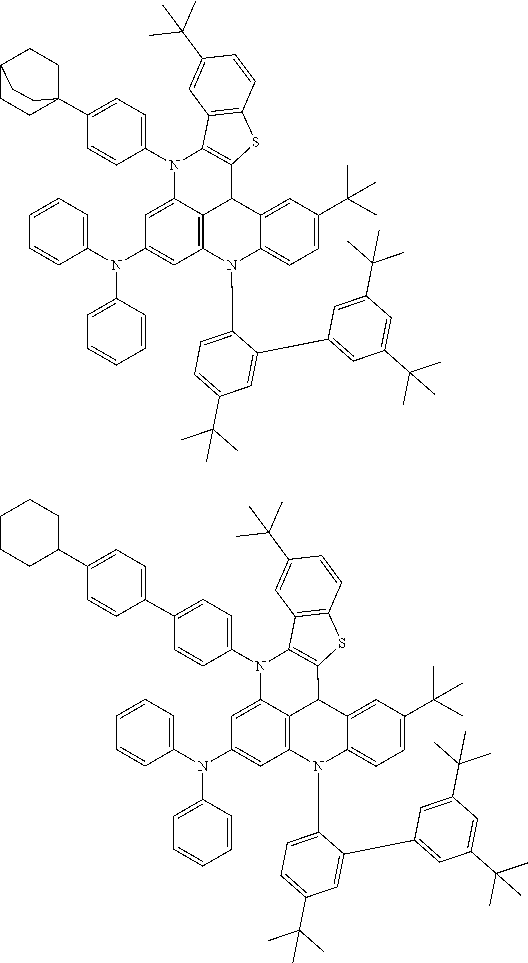 [SMILES: CC(C)(C)c1cc(-c2cc(C(C)(C)C)ccc2N2c3ccc(C(C)(C)C)cc3C3c4sc5ccc(C(C)(C)C)cc5c4N(c4ccc(C56CCC(CC5)CC6)cc4)c4cc(N(c5ccccc5)c5ccccc5)cc2c43)cc(C(C)(C)C)c1 ; CC(C)(C)c1cc(-c2cc(C(C)(C)C)ccc2N2c3ccc(C(C)(C)C)cc3C3c4sc5ccc(C(C)(C)C)cc5c4N(c4ccc(-c5ccc(C6CCCCC6)cc5)cc4)c4cc(N(c5ccccc5)c5ccccc5)cc2c43)cc(C(C)(C)C)c1]

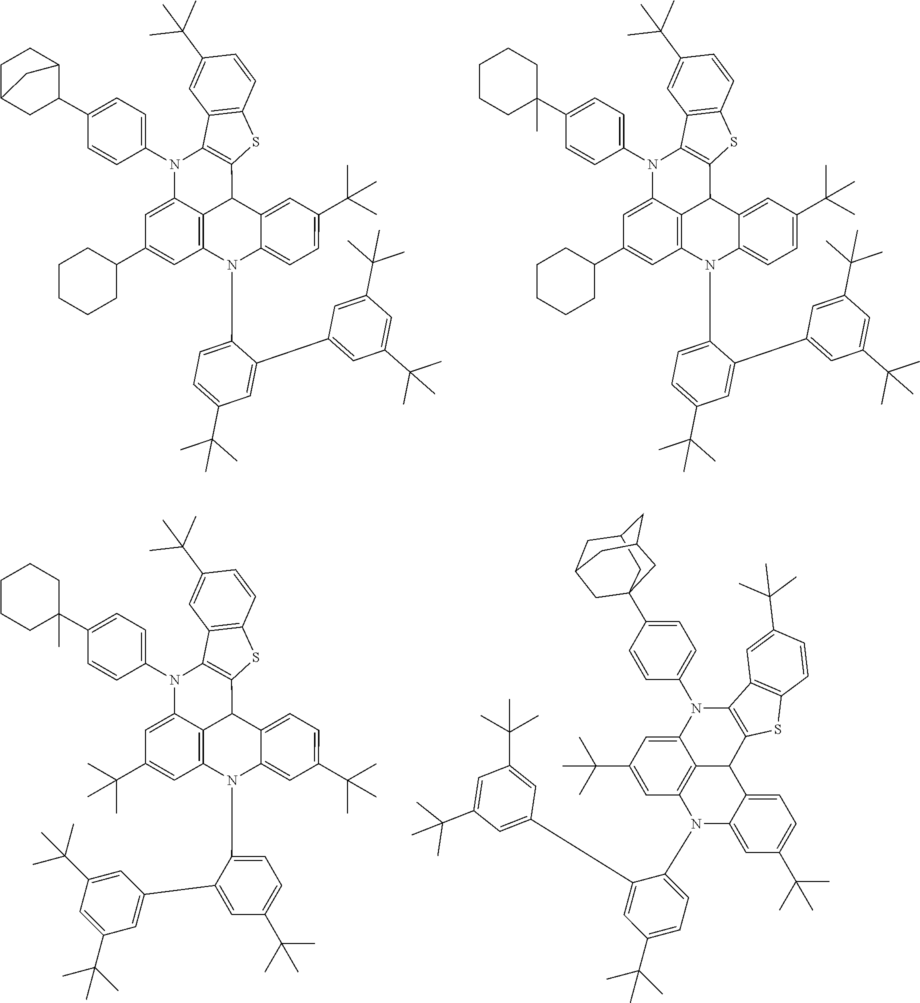

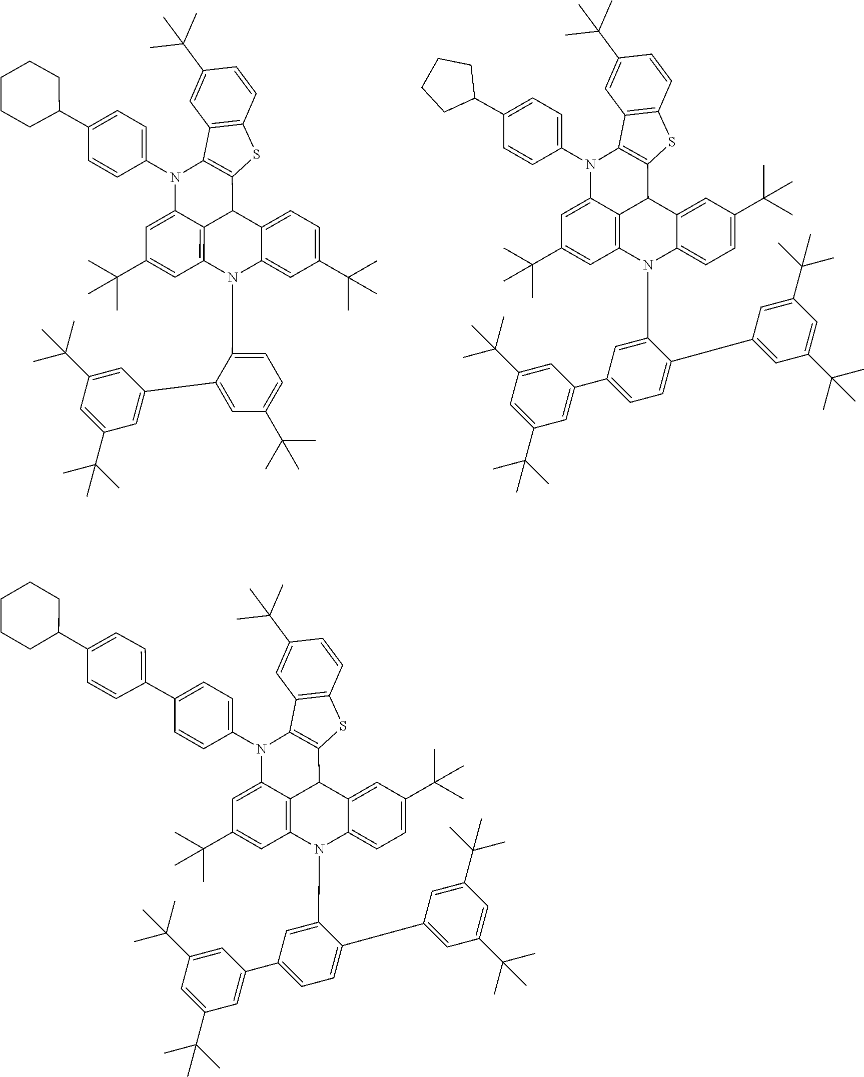 [SMILES: CC(C)(C)c1cc(-c2cc(C(C)(C)C)ccc2N2c3cc(C(C)(C)C)ccc3C3c4sc5ccc(C(C)(C)C)cc5c4N(c4ccc(C5CCCCC5)cc4)c4cc(C(C)(C)C)cc2c43)cc(C(C)(C)C)c1 ; CC(C)(C)c1cc(-c2ccc(-c3cc(C(C)(C)C)cc(C(C)(C)C)c3)c(N3c4ccc(C(C)(C)C)cc4C4c5sc6ccc(C(C)(C)C)cc6c5N(c5ccc(C6CCCC6)cc5)c5cc(C(C)(C)C)cc3c54)c2)cc(C(C)(C)C)c1 ; CC(C)(C)c1cc(-c2ccc(-c3cc(C(C)(C)C)cc(C(C)(C)C)c3)c(N3c4ccc(C(C)(C)C)cc4C4c5sc6ccc(C(C)(C)C)cc6c5N(c5ccc(-c6ccc(C7CCCCC7)cc6)cc5)c5cc(C(C)(C)C)cc3c54)c2)cc(C(C)(C)C)c1]

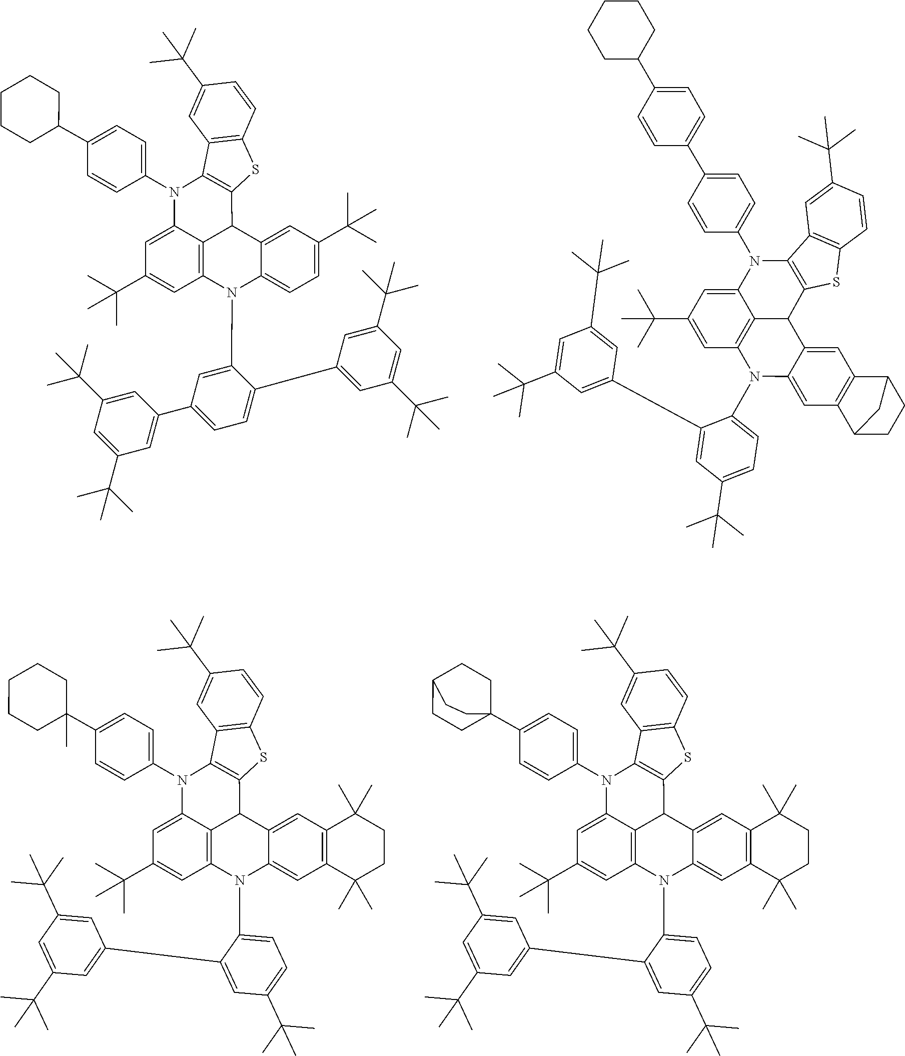

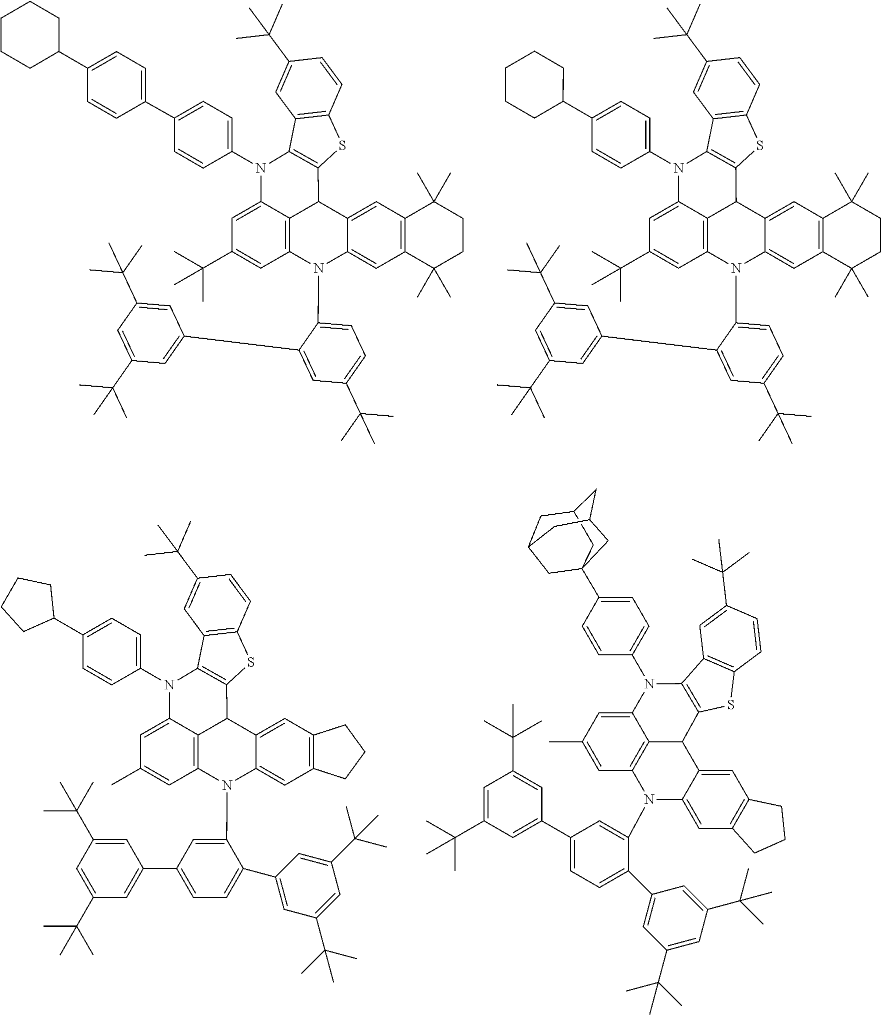

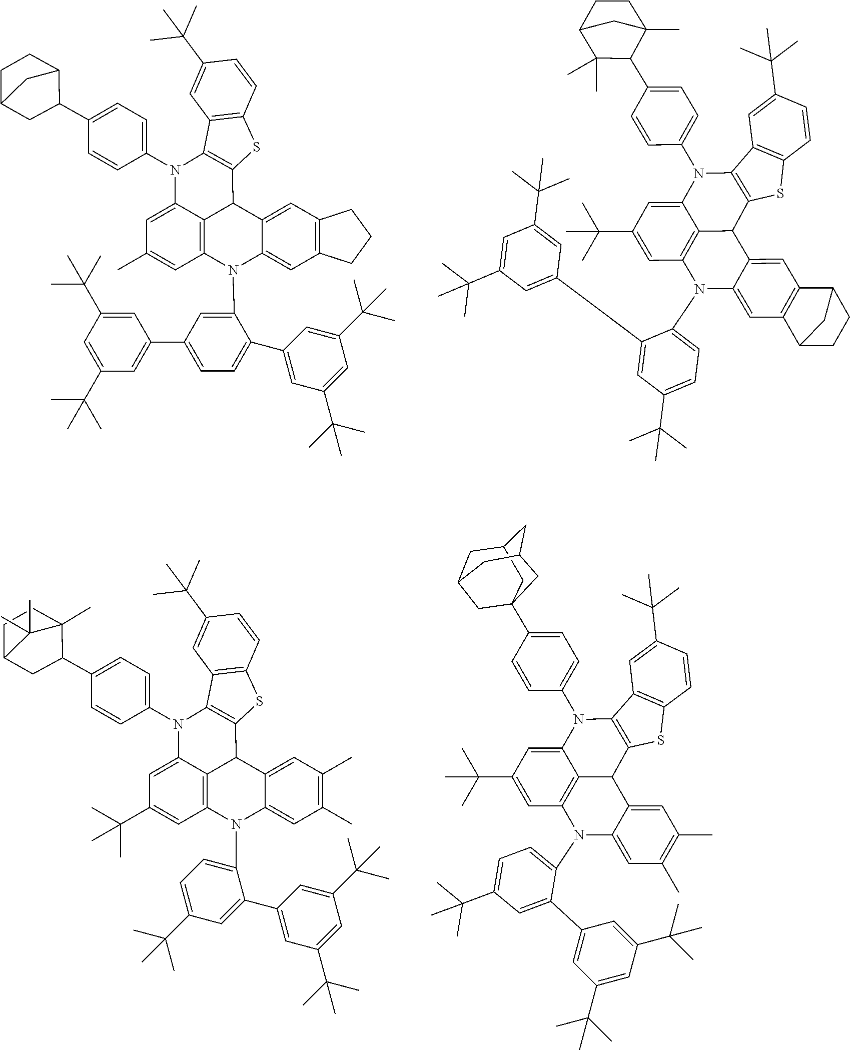

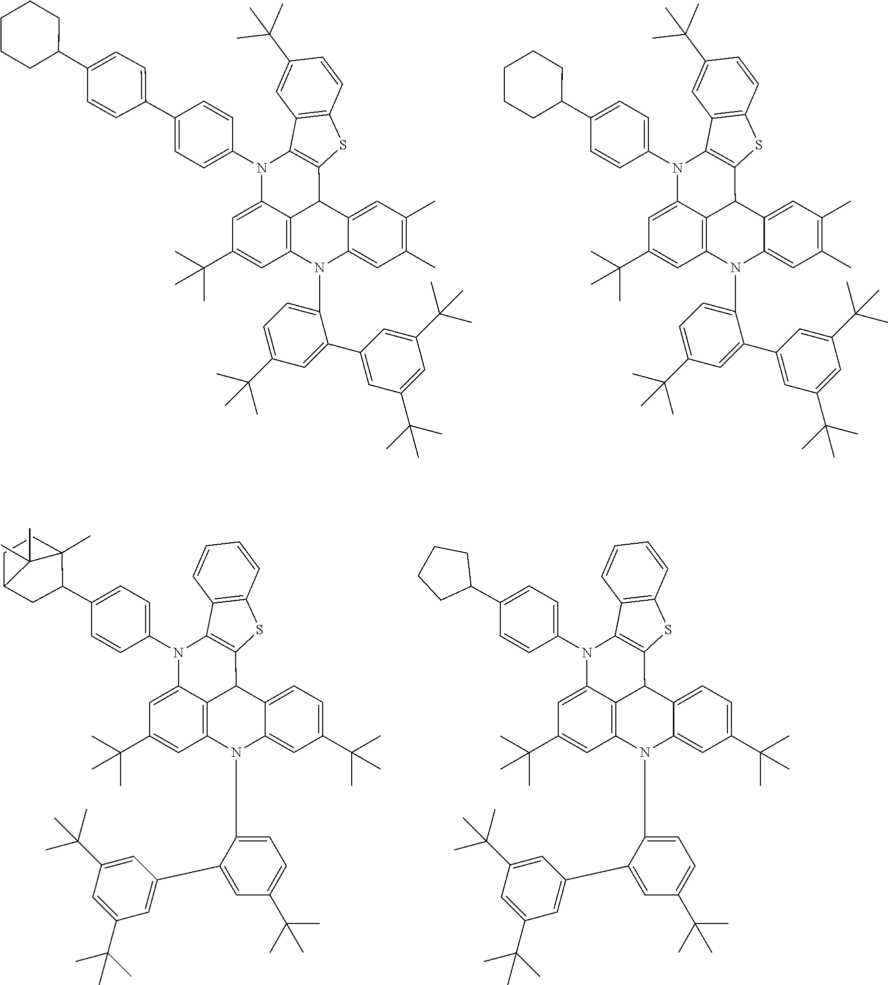

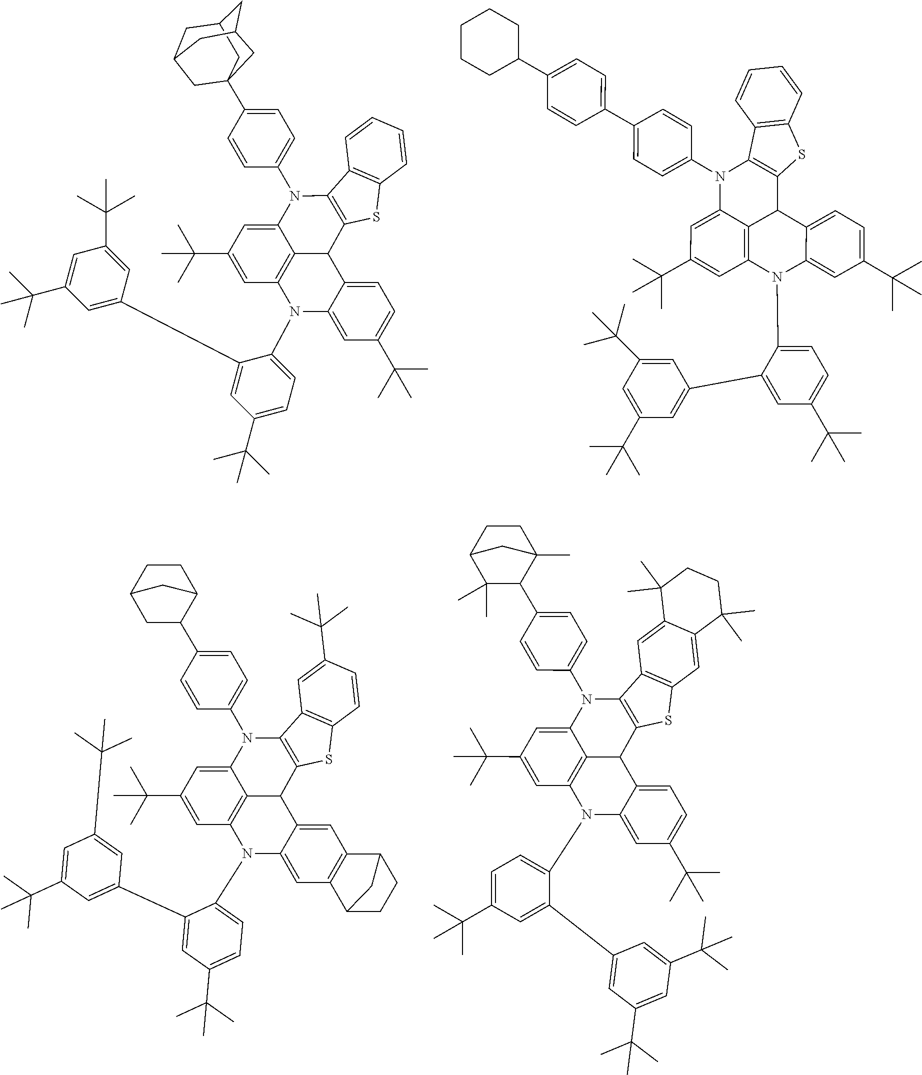

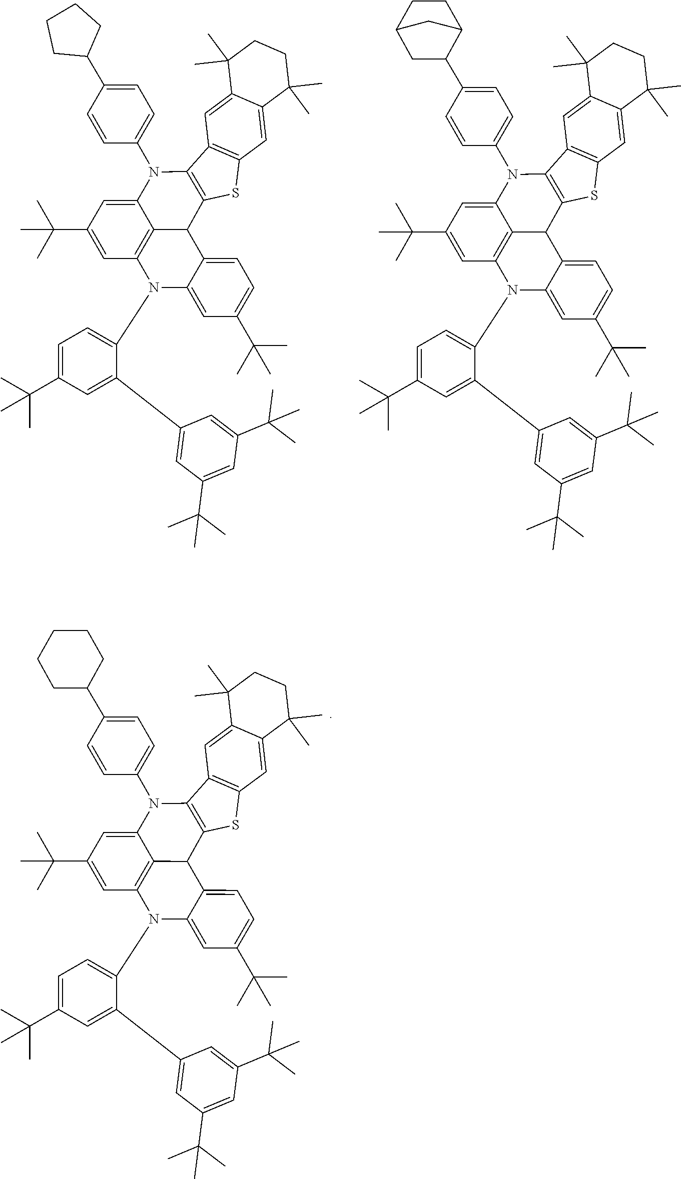 [SMILES: CC(C)(C)c1cc(-c2cc(C(C)(C)C)ccc2N2c3cc(C(C)(C)C)ccc3C3c4sc5cc6c(cc5c4N(c4ccc(C5CCCC5)cc4)c4cc(C(C)(C)C)cc2c43)C(C)(C)CCC6(C)C)cc(C(C)(C)C)c1 ; CC(C)(C)c1cc(-c2cc(C(C)(C)C)ccc2N2c3cc(C(C)(C)C)ccc3C3c4sc5cc6c(cc5c4N(c4ccc(C5CC7CCC5C7)cc4)c4cc(C(C)(C)C)cc2c43)C(C)(C)CCC6(C)C)cc(C(C)(C)C)c1 ; CC(C)(C)c1cc(-c2cc(C(C)(C)C)ccc2N2c3cc(C(C)(C)C)ccc3C3c4sc5cc6c(cc5c4N(c4ccc(C5CCCCC5)cc4)c4cc(C(C)(C)C)cc2c43)C(C)(C)CCC6(C)C)cc(C(C)(C)C)c1]

A third aspect of the present invention provides an organic electroluminescent device, comprising sequentially a first electrode disposed on a substrate plate, a second electrode disposed opposite to the first electrode, and one or more organic functional layers disposed between the first electrode and the second electrode,
wherein the organic functional layer comprises a luminescent layer comprising the composition as described above.

In conjunction with the third aspect, the luminescent layer comprises a host material and a doping material, wherein the host material comprises one or more compounds represented by Chemical Formula I and one or more compounds represented by Chemical Formula II. In particular, the host material comprises a first host material and a second host material, wherein the first host material comprises one or more compounds represented by Formula I, and the second host material comprises one or more compounds represented by Formula II.

In conjunction with the third aspect, the doping material comprises one or more compounds represented by Chemical Formula III.

### Beneficial Effects of the Invention:

The organic electroluminescent compound provided in the first aspect of the present invention has an anthracene structure to which a pyridylcarbazole fragment is connected, wherein the anthracene fragment has relatively strong conjugation and can transport both holes and electrons, and the carbazole group has a relatively strong electron-donating ability. In the present invention, introducing pyridyl into the carbazole and then connecting the pyridylcarbazole fragment to the anthracene structure can not only enhance the electron-pushing ability of the compound and adjust the electron mobility of molecules, but also make the film formation of the material more stable and prolong the lifetime of the organic electroluminescent device.

The composition provided according to the second aspect of the present invention comprises the compound represented by Formula I as described in the first aspect and further comprises a compound represented by Formula II containing a furan structure and a compound represented by Formula III containing a large steric hindrance and a thiophene structure. The compound represented by Formula I provided by the present invention contains a pyridylcarbazole structure, which has electron-pushing property and can adjust the electron cloud density on the anthracene ring so as to facilitate hole transport. The compound represented by Formula II provided by the present invention contains a furan structure, which has electron-pulling property and can adjust the electron cloud density on the anthracene ring so as to facilitate electron transport. Compared with fused cyclic anthracene-based compounds, the mixture formed by the interaction between the compound represented by Formula I and the compound represented by Formula II in the present invention is more suitable for the transport of electrons and holes and can significantly balance the carrier transport. In the composition provided by the present invention, by separately defining the structures of the compounds represented by Formulas I, II and III and by means of the interaction between the compound represented by Formula III and the compounds represented by Formulas I and II, the molecules of the compound represented by Formula III in the composition can be horizontally distributed in the mixture of the compounds represented by Formulas I and II, such that the light emitted from the compound represented by Formula III tends to be emitted in the direction perpendicular to the substrate, thereby reducing the energy loss caused during light transmission.

The third aspect of the present invention provides an organic electroluminescent device, in which a compound represented by Formula I is used as a first host compound, a compound represented by Formula II is used as a second host compound, the compound represented by Formula I and the compound represented by Formula II are mixed as a host material, and a compound represented by Formula III is used as a doping material to be applied in the luminescent layer of the organic electroluminescent device. Compared with a traditional blue light host material, the mixed host material formed by the interaction between the compound represented by Formula I and the compound represented by Formula II in the present invention is more suitable for the transport of electrons and holes and can significantly improve the balance of carriers in the blue light host material, so that the recombination center is located in the center of the luminescent layer, which can avoid the energy loss caused by the deviation of the recombination center, reduce the risk of material decomposition in adjacent layers, and can thus improve the efficiency and lifetime of the organic electroluminescent device. In the present invention, by introducing a pyridine fragment into the carbazole group in the first host compound, which pyridine can form hydrogen bonds with the molecule of the second host compound and the molecule of the doping material, the film formation becomes more stable, and the luminescence lifetime of the organic electroluminescent device can thus be improved. In the composition provided by the present invention, by separately defining the structures of the first host compound, the second host compound, and the doping material and by means of the interaction between the doping material and the first and second host compounds, the molecules of the doping material in the composition can be horizontally distributed in the mixture composed of the first host compound and the second host compound, the light emitted from the doping material tends to be emitted in the direction perpendicular to the substrate, thereby reducing the energy loss caused during light transmission in the organic electroluminescent device, thus improving the efficiency of the organic electroluminescent device. In the present invention, by means of the rational combination of the mixed host material and the doping material, the interaction of the first host compound, the second host compound, and the compound as a doping material imparts the organic electroluminescent device with the significant advantages of a high efficiency and a long lifetime. The use of the compound represented by Formula I, the compound represented by Formula II and the compound represented by Formula III provided by the present invention for use as the luminescent layer material can improve the luminous efficiency of the blue organic electroluminescent device, prolong the lifetime of the blue organic electroluminescent device and overcome the defects in the prior art.

### BRIEF DESCRIPTION OF THE DRAWINGS

The specific embodiments of the present invention will be described in further detail with reference to the accompanying drawings.

FIG. 1 is a schematic structural diagram of an organic electroluminescent device containing the compound and composition of the present invention.

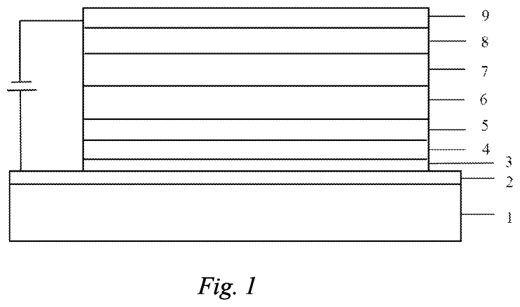

In the Brief Description of the Drawings: 1—substrate, 2—anode, 3—hole injection layer, 4—hole transport layer, 5—luminescent auxiliary layer, 6—luminescent layer, 7—electron transport layer, 8—electron injection layer, and 9—cathode.

### DETAILED DESCRIPTION OF EMBODIMENTS

In order to explain the present invention more clearly, the present invention will be further explained below in conjunction with preferred examples and the accompanying drawings. A person skilled in the art should understand that the following detailed description is illustrative rather than restrictive, and should not limit the scope of protection of the present invention. The examples and comparative examples in the present description are provided to explain the present description more completely to those skilled in the art. The examples and comparative examples according to the present description can be transformed into various forms, and the scope of protection of the present invention should not be limited to the examples and comparative examples detailed below.

The organic compound and composition of the present invention are suitable for light-emitting elements, display panels, and electronic devices, especially suitable for organic electroluminescent devices. The electronic device of the present invention is a device that comprises a layer of at least one organic compound, and the device may also comprise an inorganic material or a layer formed entirely of an inorganic material. The electronic device is preferably an organic electroluminescent device (OLED), an organic integrated circuit (O-IC), an organic field-effect transistor (O-FET), an organic thin film transistor (O-TFT), an organic light-emitting transistor (O-LET), an organic solar cell (O-SC), an organic dye-sensitized solar cell (O-DSSC), an organic optical detector, an organic photosensor, an organic field-quenching device (O-FQD), a luminescent electrochemical cell (LEC), an organic laser diode (O-laser), and an organic plasma emitting device. The electronic device is preferably an organic electroluminescent device (OLED).

In order to understand the content of the present invention more clearly, the organic compound, the preparation method for the compound, and the luminescent characteristics of the device will be explained in detail in conjunction with examples. Various chemical reactions can be applied to the synthesis method for a compound according to one embodiment of the present invention. However, it should be noted that the synthesis method for the compound according to one embodiment of the present invention is not limited to the synthesis method described below. Unless otherwise specified, the subsequent synthesis is carried out in an anhydrous solvent in a protective gas atmosphere. Solvents and reagents can be purchased from conventional reagent suppliers.

### Compound Synthesis Examples

### Synthesis of Intermediate of First Host Compound

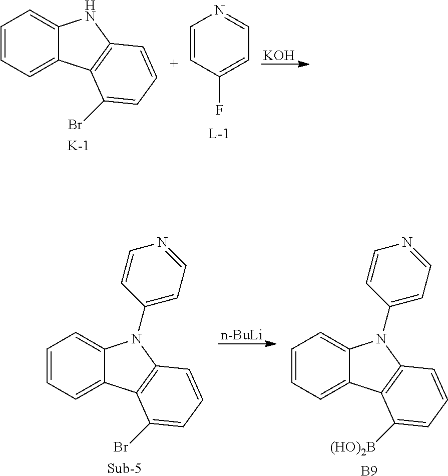

K-1 (24.6 g, 0.1 mol), L-1 (9.7 g, 0.1 mol) and potassium hydroxide (5.6 g, 0.1 mol) were added to 500 mL of dimethylformamide, heated to 80° C., stirred for 6 hours, and cooled to room temperature, 1000 mL of water was added to the reaction system, the reaction system was filtered to obtain a solid, and the solid was subjected to suction filtration to obtain a filter cake. The filter cake was recrystallized twice from a mixed solution of toluene and ethanol (the volume ratio of toluene to ethanol was 1:3) to obtain Sub-5 (24.7 g, 0.077 mol), yield 76.7%, MS (m/z) (M+): 322.2; and

Sub-5 (24.7 g, 0.077 mol) was dissolved in 250 mL of tetrahydrofuran, the mixture was cooled to −78° C. and stirred, 31 mL of n-butyl lithium (2.5 mol/L) was added to the reaction system, the reaction system was stirred at −78° C. for 1 hour, and trimethyl borate (8.32 g, 0.08 mol) was then added and stirred for 30 minutes. The reaction system was heated to 0° C., 100 mL of hydrochloric acid with a molar concentration of 1 mol/L was added, and the mixture was heated to room temperature and stirred for 1 hour. 500 mL of toluene and 500 ml of water were added to the reaction system and separated to obtain an organic phase. The organic phase was subjected to rotary evaporation to dryness to obtain a crude product. The crude product was separated and purified by column chromatography (the volume ratio of petroleum ether/ethyl acetate in a mobile phase for column chromatography was 1:1) to obtain the product B9 (16.6 g, 0.058 mol), yield: 75.3%, MS (m/z) (M+): 287.1.

After B9 was obtained from the above process, another first host compound intermediate could be prepared by a method similar to that for B9.

### Example 1

This example provided first host compound C9, and the synthesis route for this compound was as follows:

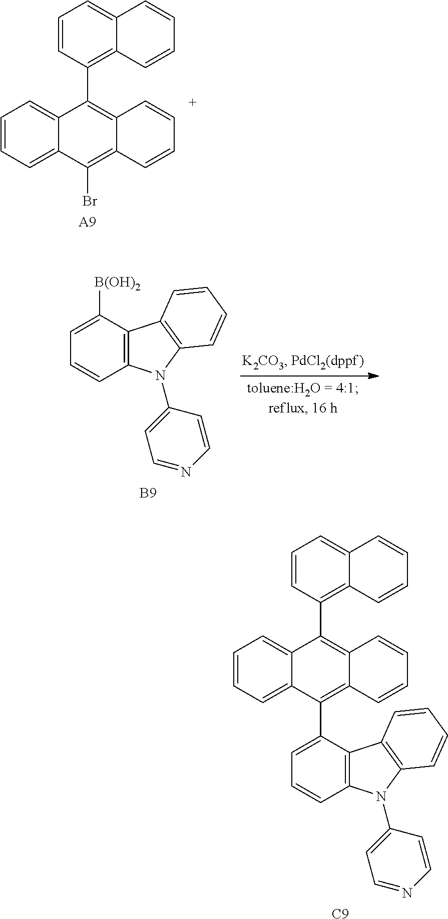

A9 (3.83 g, 10 mmol) and B9 (2.87 g, 10 mmol) were added to 100 mL of a mixed solution of toluene and water (the volume ratio of toluene to water was 4:1). Under nitrogen protection, potassium carbonate (1.65 g, 10 mmol) and [1,1′-bis(diphenylphosphino) ferrocene]dichloropalladium (II) (73 mg, 0.1 mmol) were added. The reaction system was heated to reflux for 16 hours. After cooling to room temperature, the reaction was quenched with ice water and separated to obtain an organic phase. After filtration, the organic phase was dried with anhydrous magnesium sulfate and subjected to rotary evaporation to remove the solvent to obtain a crude product. The crude product was purified by column chromatography to finally obtain the product C9: 3.00 g (yield: 55%), MS (m/z) (M+): 546.7.

### Example 2

This example provided first host compound C10, and the synthesis route for this compound was as follows:

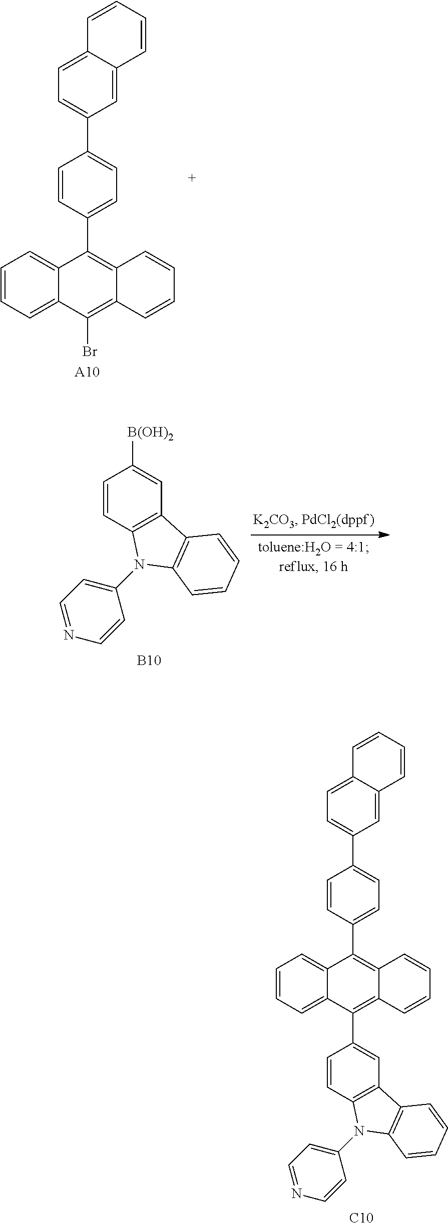

Using the same method as in Example 1, A10 (4.59 g, 10 mmol) was substituted for A9, and B10 (2.87 g, 10 mmol) was substituted for B9 to finally obtain the product C10: 3.42 g (yield: 55%), MS (m/z) (M+): 622.8.

### Example 3

This example provided first host compound C11, and the synthesis route for this compound was as follows:

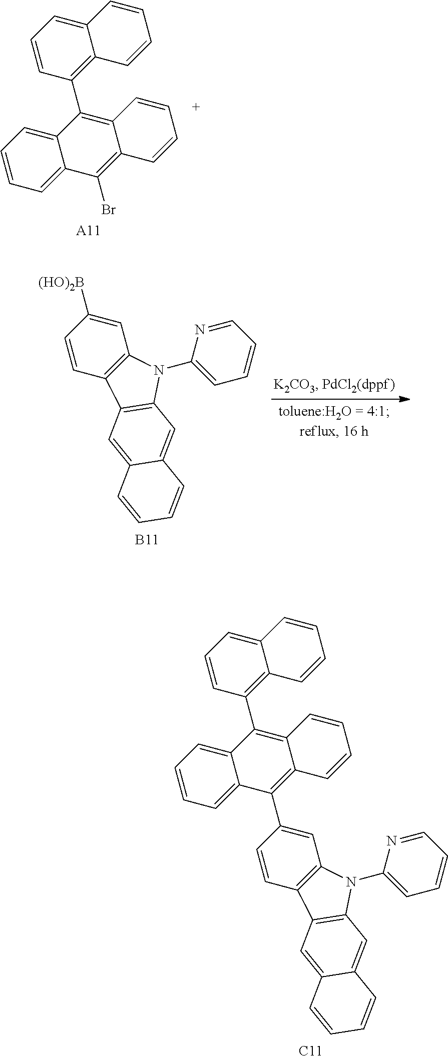

Using the same method as in Example 1, A11 (3.83 g, 10 mmol) was substituted for A9, and B11 (3.37 g, 10 mmol) was substituted for B9 to finally obtain the product C11: 3.34 g (yield: 56%), MS (m/z) (M+): 596.7.

### Example 4

This example provided first host compound C12, and the synthesis route for this compound was as follows:

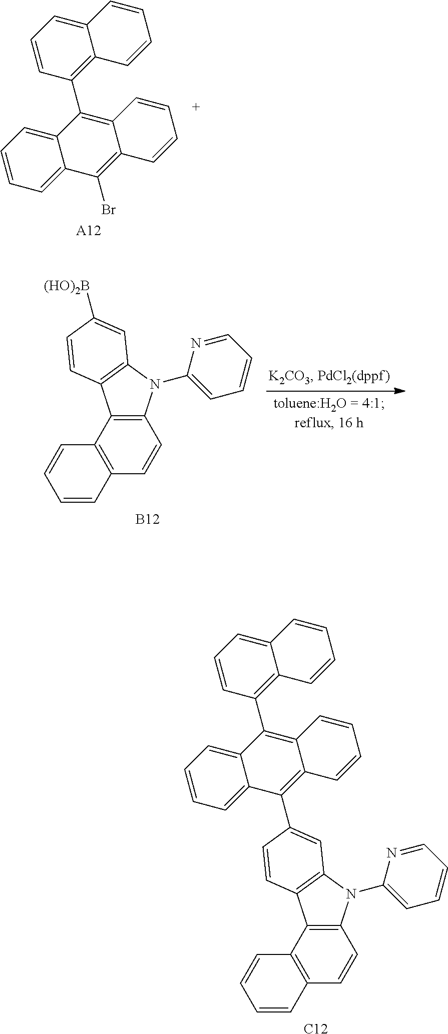

Using the same method as in Example 1, A12 (3.83 g, 10 mmol) was substituted for A9, and B12 (3.37 g, 10 mmol) was substituted for B9 to finally obtain the product C12: 4.05 g (yield: 68%), MS (m/z) (M+): 596.7.

### Example 5

This example provided first host compound C13, and the synthesis route for this compound was as follows:

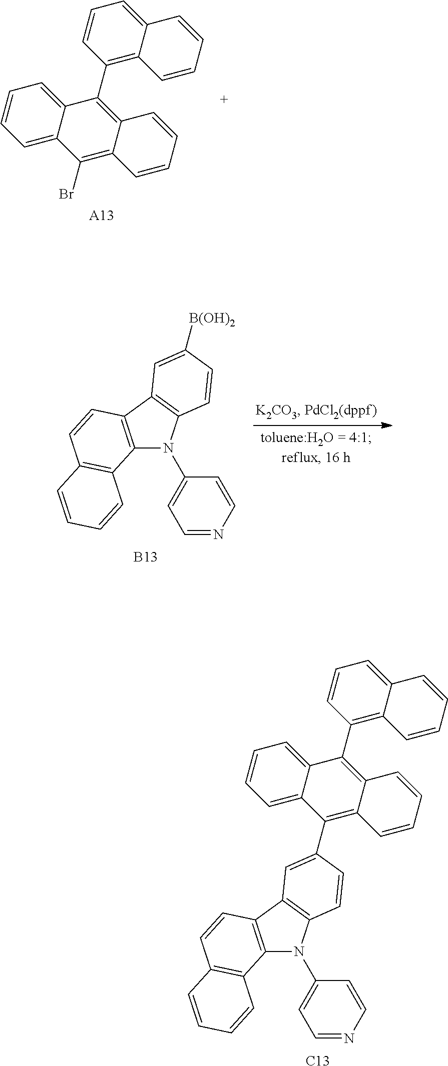

Using the same method as in Example 1, A13 (3.83 g, 10 mmol) was substituted for A9, and B13 (3.37 g, 10 mmol) was substituted for B9 to finally obtain the product C13: 3.69 g (yield: 62%), MS (m/z) (M+): 596.7.

### Example 6

This example provided first host compound C14, and the synthesis route for this compound was as follows:

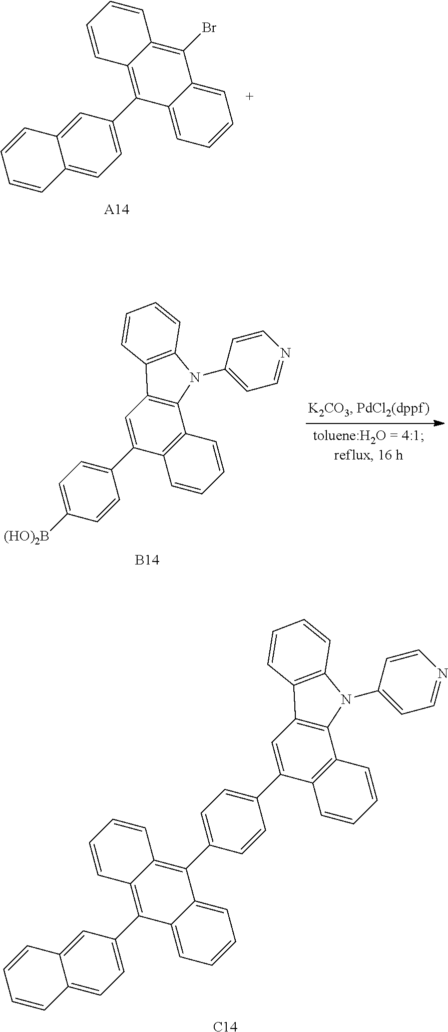

Using the same method as in Example 1, A14 (3.83 g, 10 mmol) was substituted for A9, and B14 (4.13 g, 10 mmol) was substituted for B9 to finally obtain the product C14: 3.56 g (yield: 53%), MS (m/z) (M+): 672.8.

### Example 7

This example provided second host compound C1, and the synthesis route for this compound was as follows:

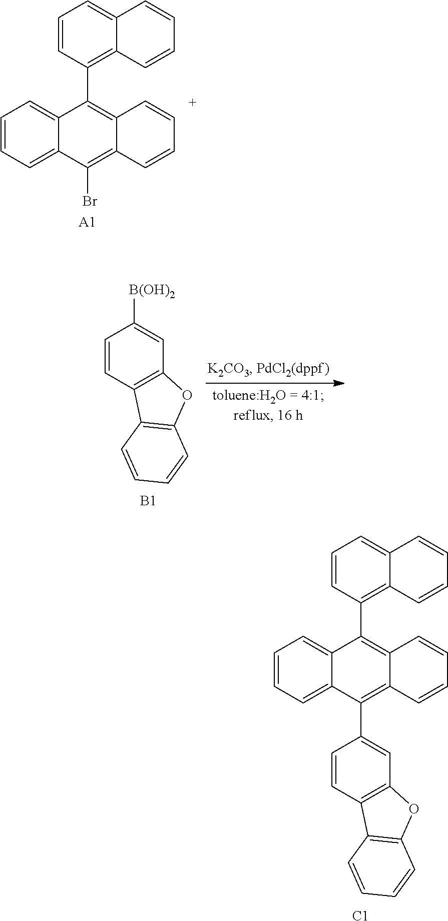

A1 (3.83 g, 10 mmol) and B1 (2.12 g, 10 mmol) were added to 100 mL of a mixed solution of toluene and water (the volume ratio of toluene to water was 4:1). Under nitrogen protection, potassium carbonate (1.65 g, 10 mmol) and [1,1′-bis(diphenylphosphino) ferrocene]dichloropalladium (II) (73 mg, 0.1 mmol) were added. The reaction system was heated to reflux for 16 hours. After cooling to room temperature, the reaction was quenched with ice water and separated to obtain an organic phase. After filtration, the organic phase was dried with anhydrous magnesium sulfate and subjected to rotary evaporation to remove the solvent to obtain a crude product. The crude product was purified by column chromatography to finally obtain the product C1: 3.05 g (yield: 65%), MS (m/z) (M+): 470.6.

### Example 8

This example provided second host compound C2, and the synthesis route for this compound was as follows:

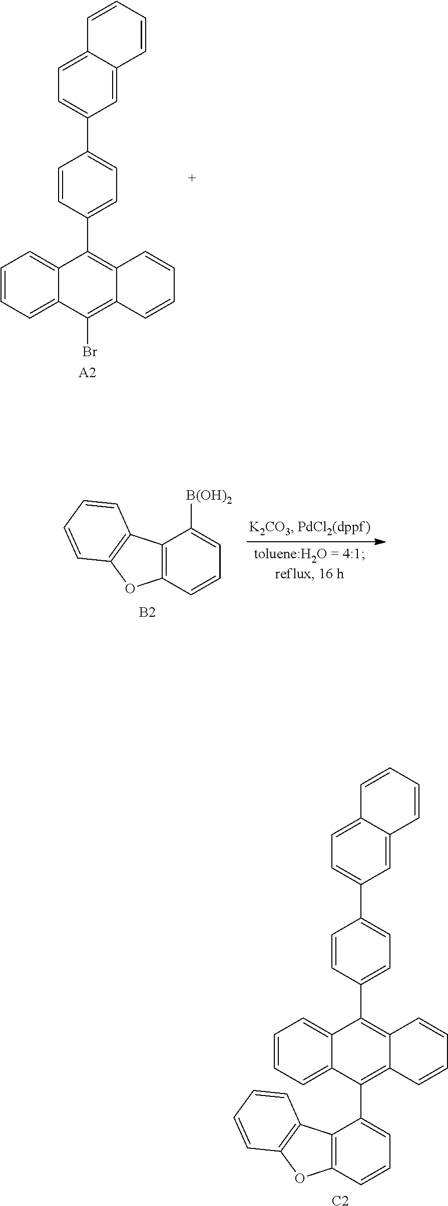

The same method as in Example 7 was used, except that A2 (4.59 g, 10 mmol) was substituted for A1, and B2 (2.12 g, 10 mmol) was substituted for B1 to finally obtain the product C2: 4.10 g (yield: 75%), MS (m/z) (M+): 546.7.

### Example 9

This example provided second host compound C3, and the synthesis route for this compound was as follows:

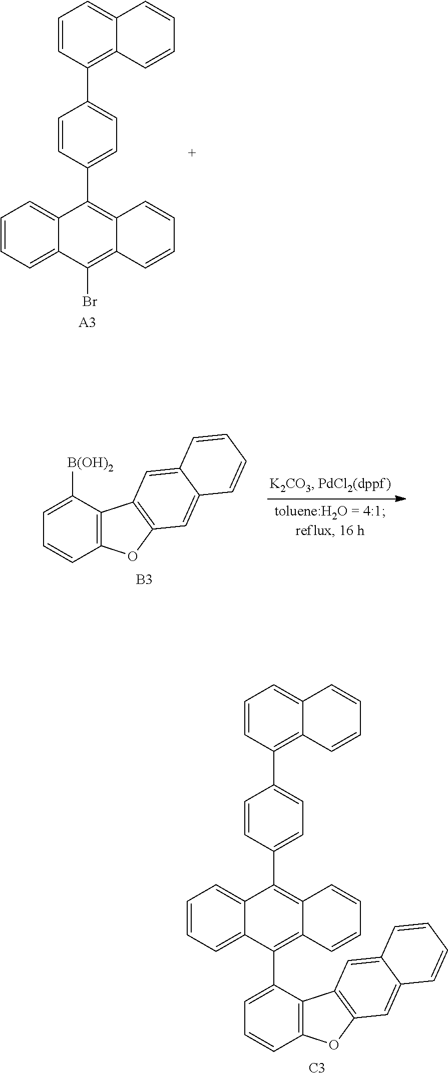

The same method as in Example 7 was used, except that A3 (4.59 g, 10 mmol) was substituted for A1, and B3 (2.62 g, 10 mmol) was substituted for B1 to finally obtain the product C3: 3.16 g (yield: 53%), MS (m/z) (M+): 596.7.

### Example 10

This example provided second host compound C4, and the synthesis route for this compound was as follows:

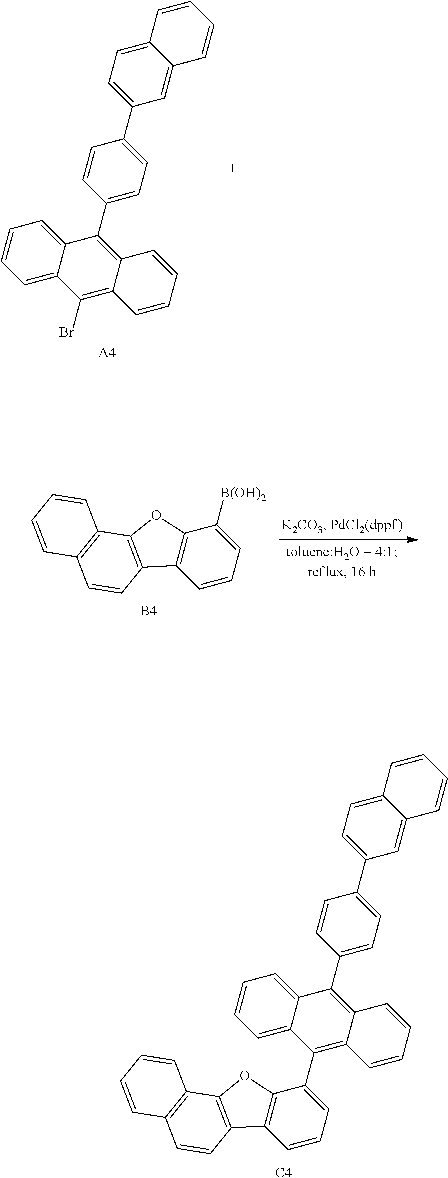

The same method as in Example 7 was used, except that A4 (4.59 g, 10 mmol) was substituted for A1, and B4 (2.62 g, 10 mmol) was substituted for B1 to finally obtain the product C4: 3.10 g (yield: 52%), MS (m/z) (M+): 596.7.

### Example 11

This example provided second host compound C5, and the synthesis route for this compound was as follows:

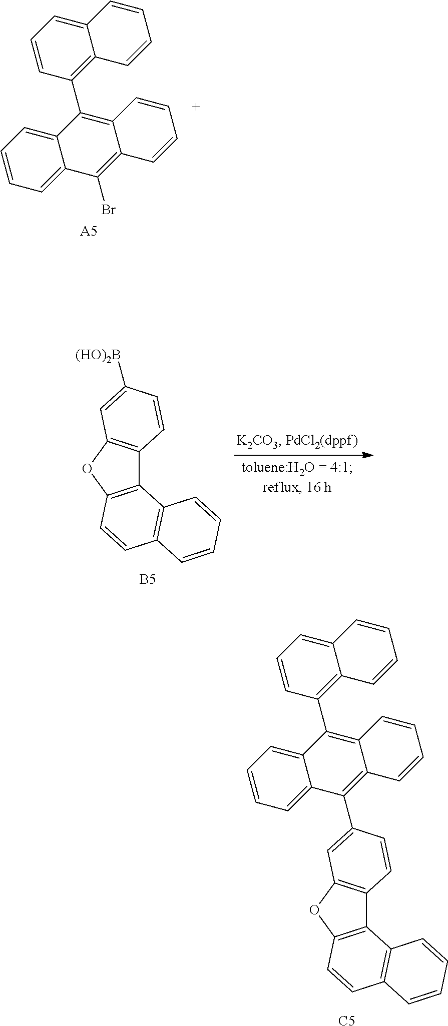

The same method as in Example 7 was used, except that A5 (3.83 g, 10 mmol) was substituted for A1, and B5 (2.62 g, 10 mmol) was substituted for B1 to finally obtain the product C5: 4.27 g (yield: 82%), MS (m/z) (M+): 520.6.

### Example 12

This example provided second host compound C6, and the synthesis route for this compound was as follows:

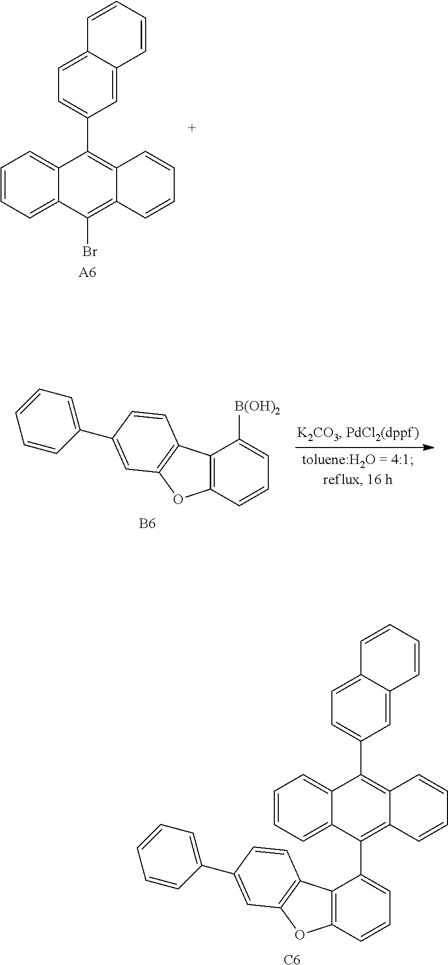

The same method as in Example 7 was used, except that A6 (3.83 g, 10 mmol) was substituted for A1, and B6 (2.88 g, 10 mmol) was substituted for B1 to finally obtain the product C6: 3.01 g (yield: 55%), MS (m/z) (M+): 546.7.

### Example 13

This example provided second host compound C7, and the synthesis route for this compound was as follows:

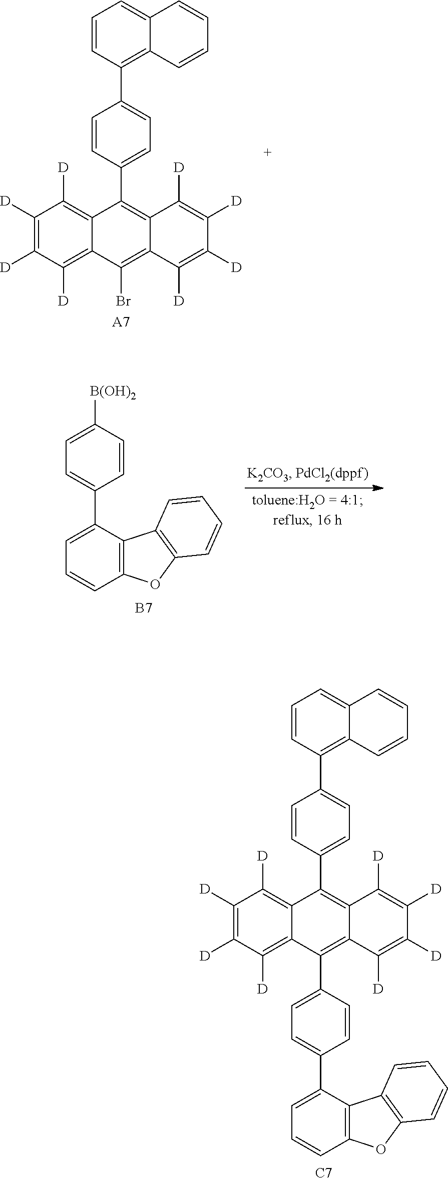

The same method as in Example 7 was used, except that A7 (4.67 g, 10 mmol) was substituted for A1, and B7 (2.88 g, 10 mmol) was substituted for B1 to finally obtain the product C7: 4.60 g (yield: 73%), MS (m/z) (M+): 630.8.

### Example 14

This example provided second host compound C8, and the synthesis route for this compound was as follows:

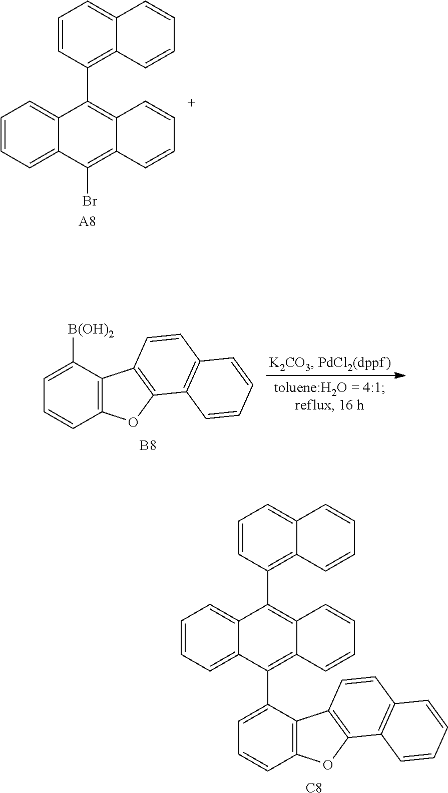

The same method as in Example 7 was used, except that A8 (3.83 g, 10 mmol) was substituted for A1, and B8 (2.62 g, 10 mmol) was substituted for B1 to finally obtain the product C8: 3.59 g (yield: 69%), MS (m/z) (M+): 520.6.

### Synthesis of Intermediate of Doping Material

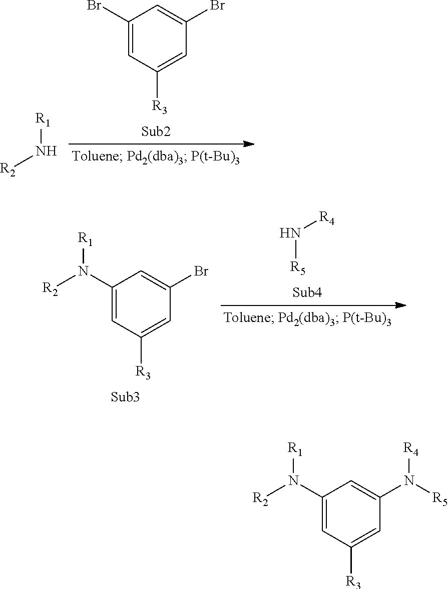

The general reaction formula of the intermediate synthesis was as above. The target intermediate could be obtained by means of a common two-step Buchwald-Hartwig coupling synthesis method.

A specific example was as follows:

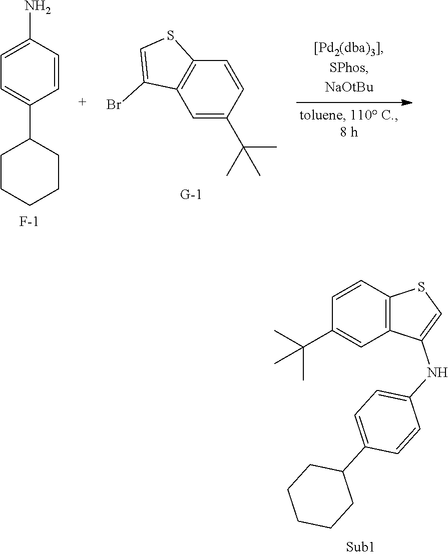

F-1 (8.75 g, 0.05 mol), G-1 (12.11 g, 0.045 mol), and sodium tert-butoxide (5.28 g, 0.055 mol) were added to toluene (200 mL). Subsequently, under nitrogen protection, bis(dibenzylideneacetone) palladium (0.46 g, 0.5 mmol) and SPhos (0.41 g, 1 mmol) were introduced. Subsequently, the reaction system was heated to 110° C., refluxed for 8 hours, and cooled to room temperature, the reaction was then quenched by adding water and separated to obtain an organic phase, the organic phase was filtered, then dried with anhydrous magnesium sulfate, and subjected to rotary evaporation to remove the solvent to obtain a crude product, and the crude product was purified by column chromatography to obtain the product Sub1: 11.47 g, yield: 70%, MS (m/z) (M+): 364.

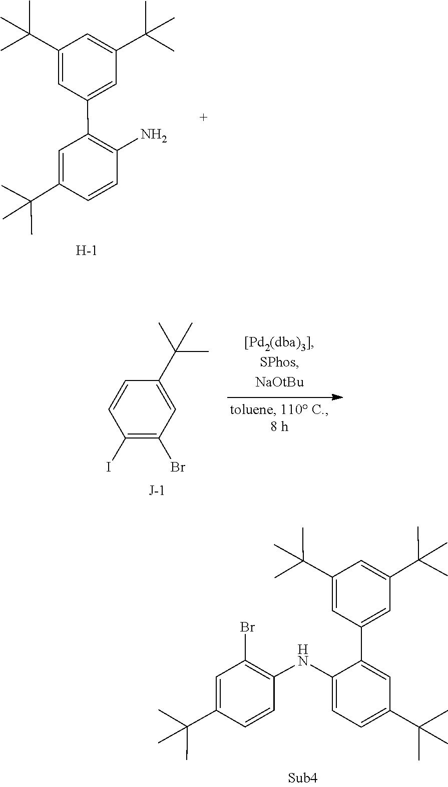

H-1 (16.9 g, 0.05 mol), J-1 (15.26 g, 0.045 mol), and sodium tert-butoxide (5.28 g, 0.055 mol) were added to toluene (500 mL). Under nitrogen protection, bis(dibenzylideneacetone) palladium (0.46 g, 0.5 mmol) and SPhos (0.41 g, 1 mmol) were introduced. Subsequently, the reaction system was heated to 110° C. and refluxed for 8 hours, and cooled to room temperature, and the reaction was then quenched by adding water and separated to obtain an organic phase. The organic phase was filtered, then dried with anhydrous sodium sulfate, and subjected to rotary evaporation to remove the solvent to obtain a crude product, and the crude product was purified by column chromatography to obtain the product Sub4: 20.01 g, yield: 81%, MS (m/z) (M+): 549.

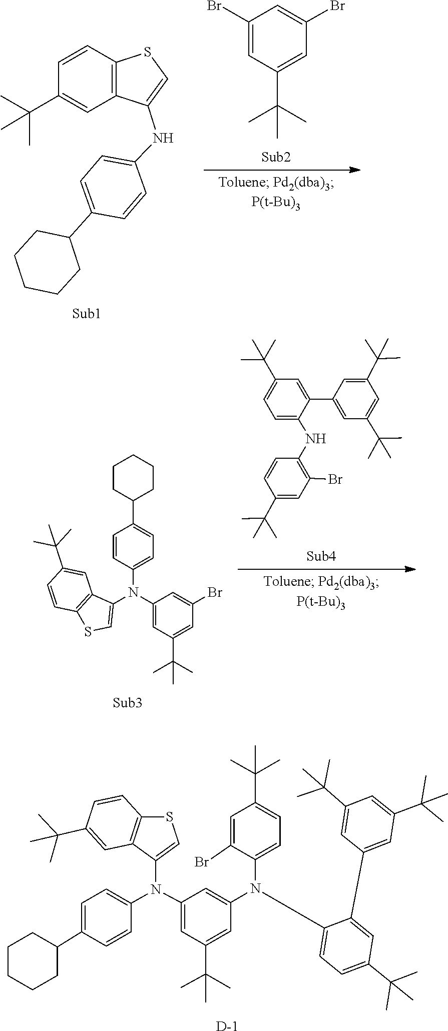

Sub1 (3.64 g, 10 mmol), Sub2 (2.63 g, 9 mmol), and sodium tert-butoxide (1.05 g, 11 mmol) were added to toluene (50 ml). Under nitrogen protection, bis(dibenzylideneacetone) palladium (274.28 mg, 0.30 mmol) and tri-tert-butylphosphine (121.2 mg, 0.6 mmol) were then introduced. Subsequently, the reaction system was heated to reflux for 10 hours and cooled to room temperature, the reaction was then quenched by adding water and separated to obtain an organic phase, the organic phase was filtered, then dried with anhydrous sodium sulfate, and subjected to rotary evaporation to remove the solvent to obtain a crude product, and the crude product was purified by column chromatography to finally obtain the product Sub3: 4.14 g (yield: 80%), MS (m/z) (M+): 575.

Sub3 (5.75 g, 10 mmol), Sub4 (5.49 g, 10 mmol), and sodium tert-butoxide (1.05 g, 11 mmol) were added to toluene (100 ml). Under nitrogen protection, bis(dibenzylideneacetone) palladium (274.28 mg, 0.30 mmol) and tri-tert-butylphosphine (121.2 mg, 0.6 mmol) were introduced. Subsequently, the reaction system was heated to reflux for 10 hours and cooled to room temperature, the reaction was then quenched by adding water and separated to obtain an organic phase, the organic phase was filtered, then dried with anhydrous sodium sulfate, and subjected to rotary evaporation to remove the solvent to obtain a crude product, and the crude product was purified by column chromatography to finally obtain the product D1: 6.77 g (yield: 65%), MS (m/z) (M+): 1042.

After D1 was obtained from the above process, another doping material intermediate could be prepared by a method similar to that for D1.

### Example 15

This example provided Compound E1 as a doping material, and the synthesis route for this compound was as follows:

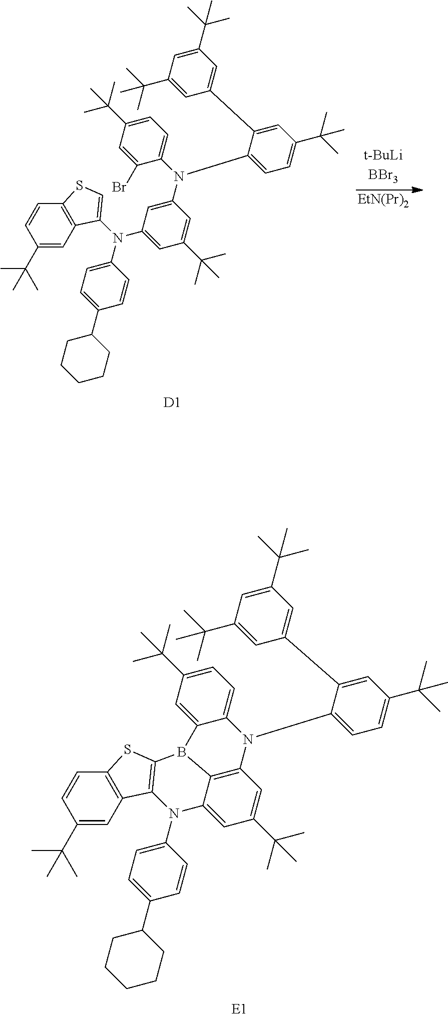

D1 (10.42 g, 10 mmol) was added to tert-butyl benzene (125 mL), the mixture was then cooled to 0° C. under nitrogen protection, and 12.4 mL of a solution of 1.7 M tert-butyl lithium (21 mmol) in pentane was added. The mixture was heated to 60° C., stirred for 2 hours, and then cooled to 0° C., and 2.0 mL of boron tribromide (21 mmol) was added and stirred for 0.5 h. At 0° C., 3.65 mL of N,N-diisopropylethylamine (21 mmol) was then added. The mixture was heated to 60° C., stirred for 2 h, and cooled to room temperature, and the reaction was then quenched by adding ice water and separated to obtain an organic phase. The organic phase was filtered, then dehydrated with anhydrous magnesium sulfate, and subjected to rotary evaporation to remove the organic solvent to obtain a crude product, and the crude product was recrystallized and purified from dichloromethane and n-heptane to finally obtain the product E1: 1.46 g (yield: 15%), MS (m/z) (M+): 971.

### Example 16

This example provided Compound E2 as a doping material, and the synthesis route for this compound was as follows:

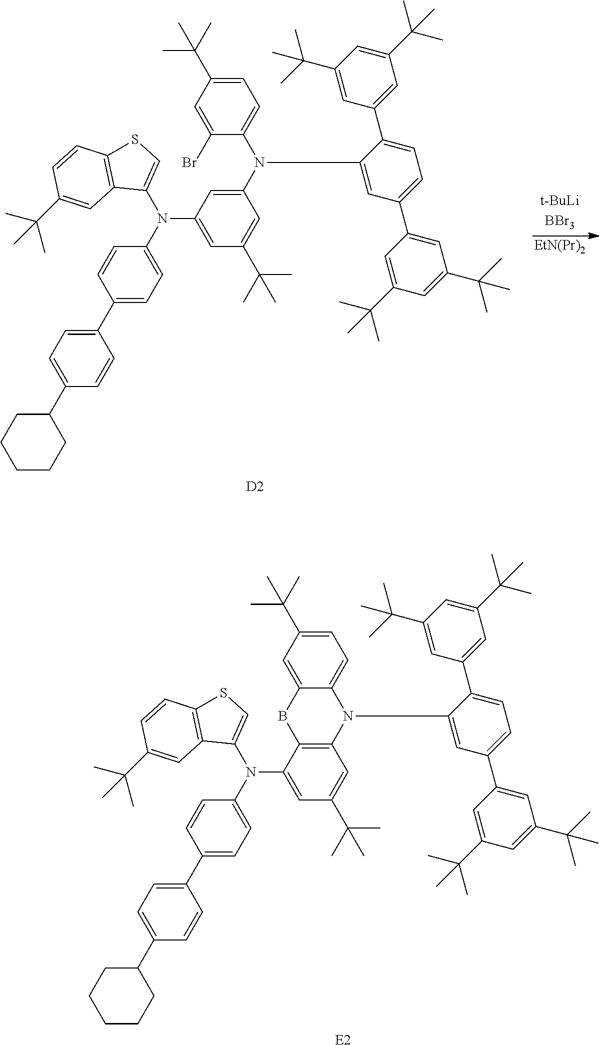

The same method as in Example 15 was used, except that D2 (12.51 g, 10 mmol) was substituted for D1 to finally obtain the product E2: 0.87 g (yield: 73.8%), MS (m/z) (M+): 1179.6.

### Example 17

This example provided Compound E3 as a doping material, and the synthesis route for this compound was as follows:

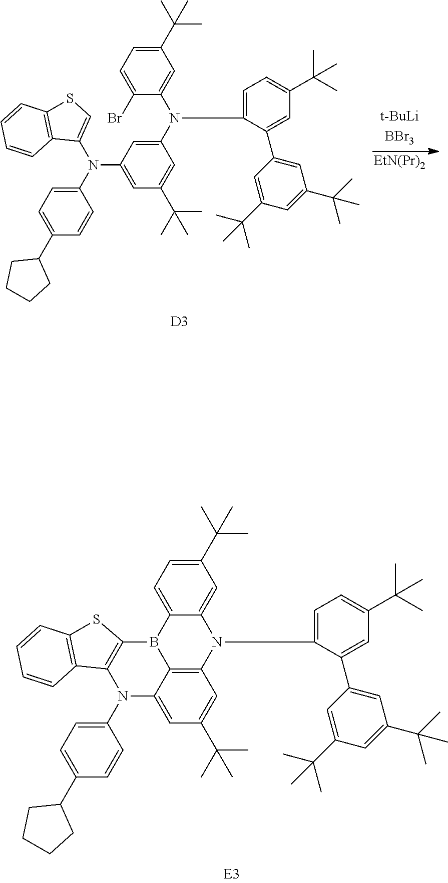 [SMILES: "EtN(Pr)2" ; CC(C)(C)c1cc(N(c2cc(C(C)(C)C)ccc2Br)c2ccc(C(C)(C)C)cc2-c2cc(C(C)(C)C)cc(C(C)(C)C)c2)cc(N(c2ccc(C3CCCC3)cc2)c2csc3ccccc23)c1 ; "D3" ; CC(C)(C)c1cc(-c2cc(C(C)(C)C)ccc2N2c3cc(C(C)(C)C)ccc3B3c4sc5ccccc5c4N(c4ccc(C5CCCC5)cc4)c4cc(C(C)(C)C)cc2c43)cc(C(C)(C)C)c1]

The same method as in Example 15 was used, except that D3 (9.72 g, 10 mmol) was substituted for D1 to finally obtain the product E3: 0.34 g (yield: 37.2%), MS (m/z) (M+): 901.2.

### Example 18

This example provided Compound E4 as a doping material, and the synthesis route for this compound was as follows:

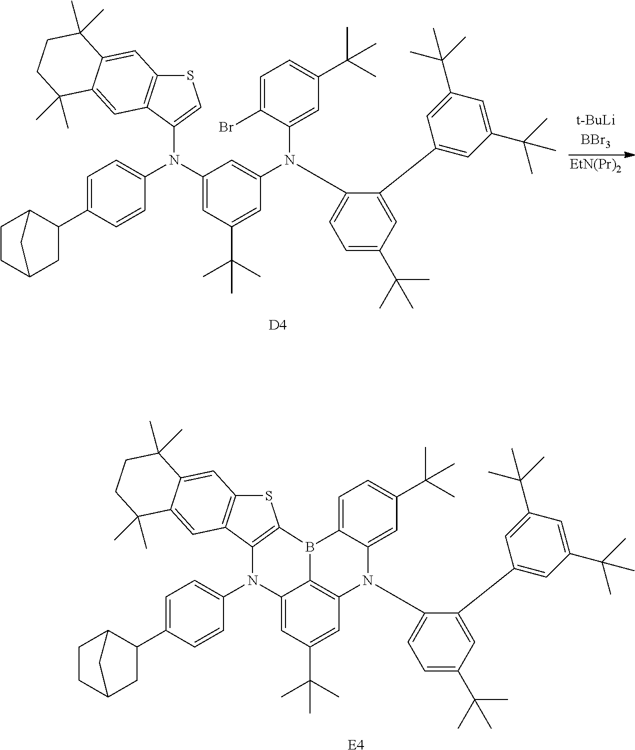

The same method as in Example 15 was used, except that D4 (11.09 g, 10 mmol) was substituted for D1 to finally obtain the product E4: 0.72 g (yield: 6.9%), MS (m/z) (M+): 1037.4.

### Example 19

This example provided Compound E5 as a doping material, and the synthesis route for this compound was as follows:

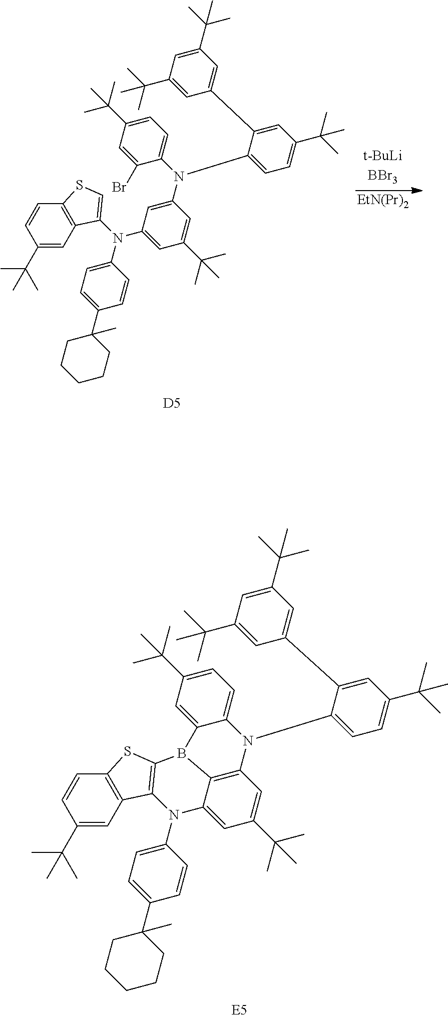

The same method as in Example 15 was used, except that D5 (10.56 g, 10 mmol) was substituted for D1 to finally obtain the product E5: 0.53 g (yield: 5.41%), MS (m/z) (M+): 985.3.

### Example 20

This example provided Compound E6 as a doping material, and the synthesis route for this compound was as follows:

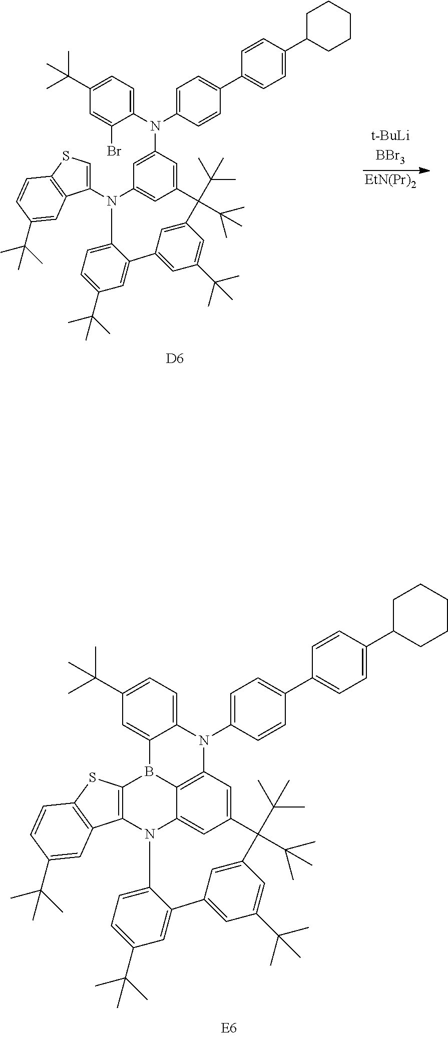 [SMILES: "EtN(Pr)2" ; CC(C)(C)c1cc2cc(c1)C(C(C)(C)C)(C(C)(C)C)c1cc(N(c3ccc(-c4ccc(C5CCCCC5)cc4)cc3)c3ccc(C(C)(C)C)cc3Br)cc(c1)N(c1csc3ccc(C(C)(C)C)cc13)c1ccc(C(C)(C)C)cc1-2 ; "D6" ; CC(C)(C)c1cc2cc(c1)C(C(C)(C)C)(C(C)(C)C)c1cc3c4c(c1)N(c1ccc(C(C)(C)C)cc1-2)c1c(sc2ccc(C(C)(C)C)cc12)B4c1cc(C(C)(C)C)ccc1N3c1ccc(-c2ccc(C3CCCCC3)cc2)cc1]

The same method as in Example 15 was used, except that D6 (11.19 g, 10 mmol) was substituted for D1 to finally obtain the product E6: 0.69 g (yield: 6.56%), MS (m/z) (M+): 1047.4.

### Example 21

This example provided Compound E7 as a doping material, and the synthesis route for this compound was as follows:

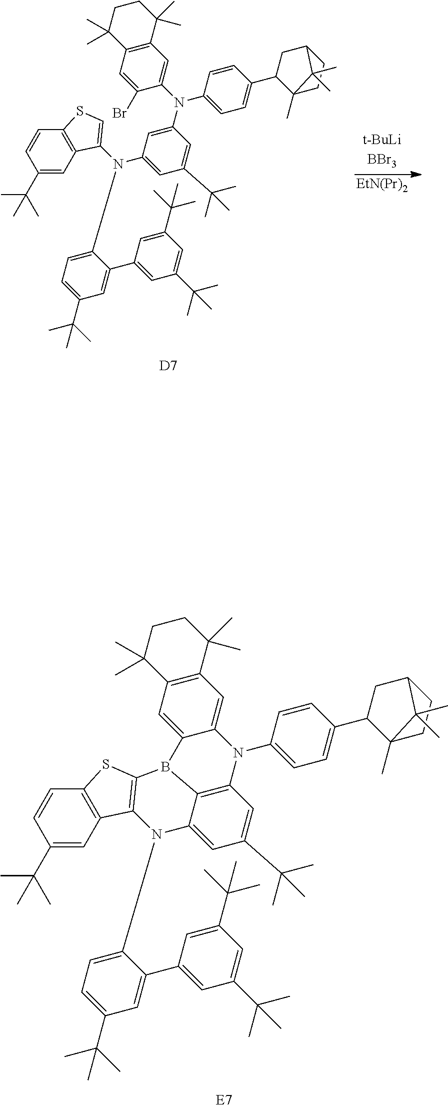

The same method as in Example 15 was used, except that D7 (11.51 g, 10 mmol) was substituted for D1 to finally obtain the product E7: 0.83 g (yield: 7.65%), MS (m/z) (M+): 1079.5.

### Example 22

This example provided Compound E8 as a doping material, and the synthesis route for this compound was as follows:

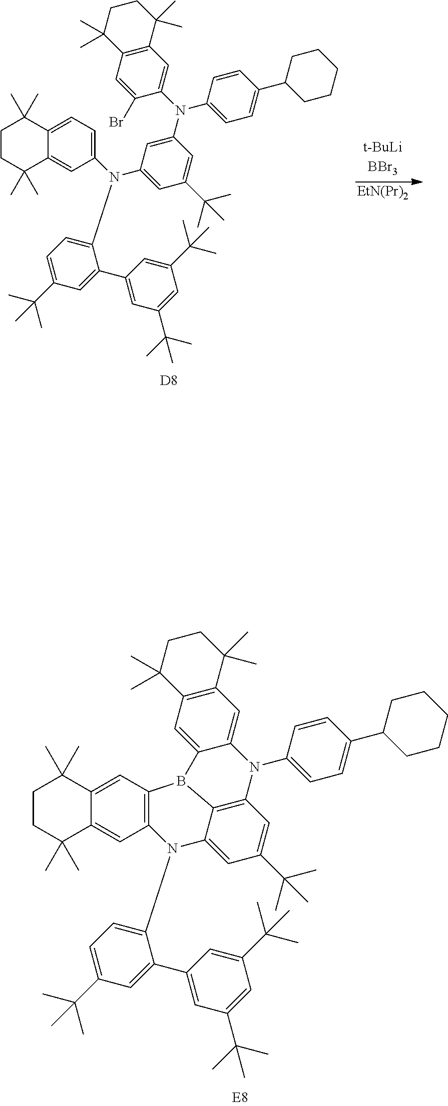

The same method as in Example 15 was used, except that D8 (10.95 g, 10 mmol) was substituted for D1 to finally obtain the product E8: 0.38 g (yield: 3.73%), MS (m/z) (M+): 1023.4.

### Example 23

This example provided Compound E9 as a doping material, and the synthesis route for this compound was as follows:

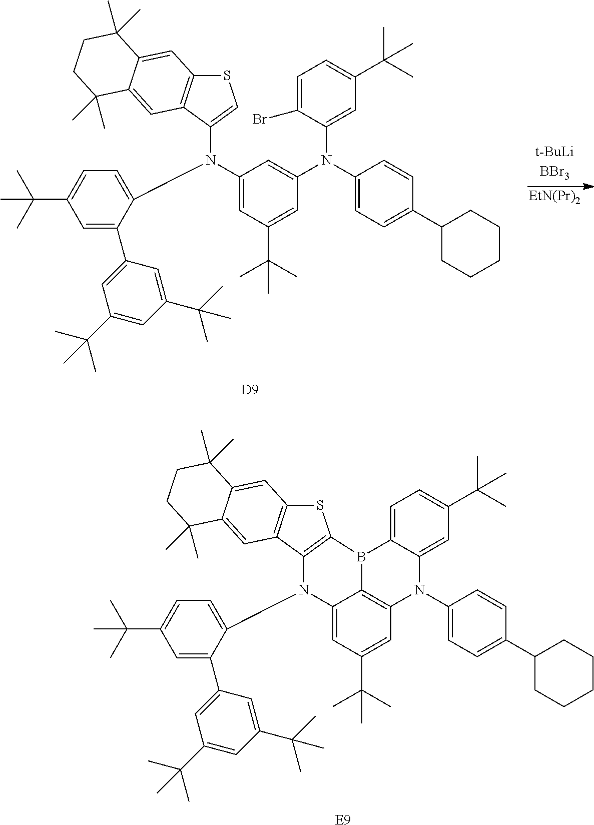

The same method as in Example 15 was used, except that D9 (10.97 g, 10 mmol) was substituted for D1 to finally obtain the product E9: 0.32 g (yield: 3.15%), MS (m/z) (M+): 1025.4.

### Example 24

This example provided Compound E10 as a doping material, and the synthesis route for this compound was as follows:

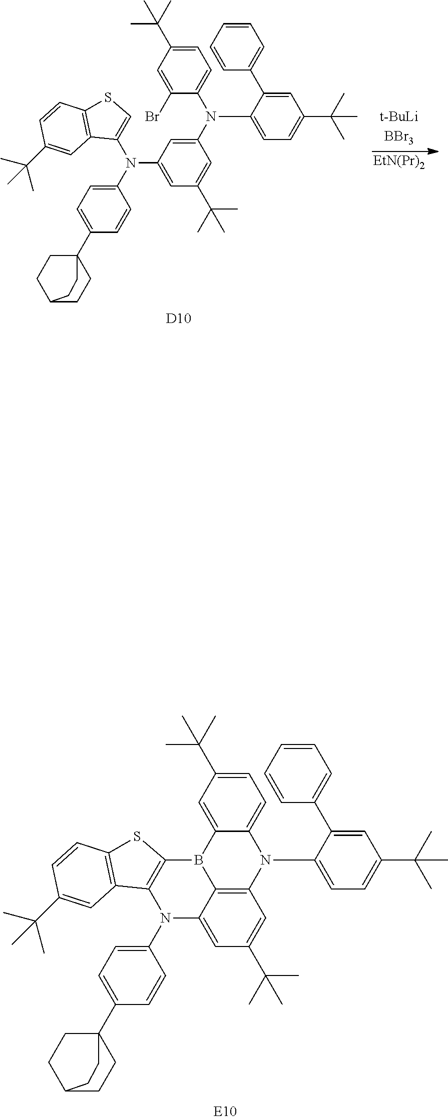

The same method as in Example 15 was used, except that D10 (9.56 g, 10 mmol) was substituted for D1 to finally obtain the product E10: 0.58 g (yield: 6.51%), MS (m/z) (M+): 885.1.

### COMPOSITION EXAMPLES

### Examples 25 to 112

Compositions T1 to T88 were obtained by combining the first host compound, the second host compound and a compound as the doping material according to Table 1 and uniformly mixing these materials, wherein the mass ratio of the first host compound to the second host compound to the compound as the doping material was 49:49:2.

TABLE 1

| | | First host | Second host | Doping |
|---|---|---|---|---|
| Example | Composition | compound | compound | material |
| 25 | T1 | C11 | C4 | E10 |
| 26 | T2 | C12 | C8 | E7 |
| 27 | T3 | C13 | C4 | E9 |
| 28 | T4 | C12 | C1 | E8 |
| 29 | T5 | C11 | C6 | E4 |
| 30 | T6 | C13 | C7 | E9 |
| 31 | T7 | C9 | C7 | E5 |
| 32 | T8 | C10 | C7 | E10 |
| 33 | T9 | C12 | C1 | E3 |
| 34 | T10 | C10 | C8 | E1 |
| 35 | T11 | C14 | C3 | E7 |
| 36 | T12 | C10 | C7 | E9 |
| 37 | T13 | C10 | C8 | E5 |
| 38 | T14 | C9 | C6 | E2 |
| 39 | T15 | C14 | C3 | E4 |
| 40 | T16 | C12 | C4 | E8 |
| 41 | T17 | C13 | C8 | E9 |
| 42 | T18 | C10 | C8 | E7 |
| 43 | T19 | C9 | C1 | E3 |
| 44 | T20 | C14 | C5 | E2 |
| 45 | T21 | C14 | C6 | E8 |
| 46 | T22 | C12 | C2 | E8 |
| 47 | T23 | C14 | C5 | E7 |
| 48 | T24 | C14 | C5 | E10 |
| 49 | T25 | C13 | C7 | E6 |
| 50 | T26 | C9 | C7 | E10 |
| 51 | T27 | C11 | C6 | E7 |
| 52 | T28 | C13 | C4 | E4 |
| 53 | T29 | C9 | C4 | E2 |
| 54 | T30 | C12 | C4 | E7 |
| 55 | T31 | C12 | C5 | E1 |
| 56 | T32 | C9 | C8 | E7 |
| 57 | T33 | C9 | C4 | E9 |
| 58 | T34 | C14 | C2 | E9 |
| 59 | T35 | C12 | C3 | E4 |
| 60 | T36 | C13 | C7 | E2 |
| 61 | T37 | C12 | C5 | E7 |
| 62 | T38 | C14 | C3 | E3 |
| 63 | T39 | C12 | C4 | E6 |
| 64 | T40 | C13 | C8 | E5 |
| 65 | T41 | C9 | C2 | E3 |
| 66 | T42 | C14 | C8 | E10 |
| 67 | T43 | C11 | C4 | E7 |
| 68 | T44 | C14 | C3 | E6 |
| 69 | T45 | C10 | C8 | E9 |
| 70 | T46 | C12 | C5 | E3 |
| 71 | T47 | C9 | C7 | E3 |
| 72 | T48 | C9 | C6 | E8 |
| 73 | T49 | C12 | C8 | E2 |
| 74 | T50 | C9 | C8 | E3 |
| 75 | T51 | C14 | C3 | E5 |
| 76 | T52 | C9 | C1 | E9 |
| 77 | T53 | C13 | C5 | E10 |
| 78 | T54 | C12 | C1 | E6 |
| 79 | T55 | C9 | C6 | E1 |
| 80 | T56 | C13 | C2 | E9 |
| 81 | T57 | C9 | C4 | E10 |
| 82 | T58 | C14 | C6 | E2 |
| 83 | T59 | C11 | C4 | E8 |
| 84 | T60 | C14 | C2 | E2 |
| 85 | T61 | C14 | C7 | E1 |
| 86 | T62 | C9 | C7 | E9 |
| 87 | T63 | C10 | C3 | E2 |
| 88 | T64 | C14 | C2 | E1 |
| 89 | T65 | C12 | C8 | E4 |
| 90 | T66 | C14 | C8 | E7 |
| 91 | T67 | C14 | C5 | E5 |
| 92 | T68 | C12 | C3 | E10 |
| 93 | T69 | C14 | C8 | E3 |
| 94 | T70 | C9 | C7 | E7 |
| 95 | T71 | C9 | C7 | E2 |
| 96 | T72 | C9 | C7 | E6 |
| 97 | T73 | C10 | C4 | E9 |
| 98 | T74 | C11 | C8 | E5 |
| 99 | T75 | C14 | C7 | E9 |
| 100 | T76 | C12 | C8 | E8 |
| 101 | T77 | C9 | C2 | E7 |
| 102 | T78 | C12 | C1 | E5 |
| 103 | T79 | C9 | C1 | E10 |
| 104 | T80 | C13 | C5 | E6 |
| 105 | T81 | C10 | C4 | E5 |
| 106 | T82 | C11 | C4 | E4 |
| 107 | T83 | C9 | C6 | E7 |
| 108 | T84 | C13 | C4 | E8 |
| 109 | T85 | C13 | C5 | E3 |
| 110 | T86 | C13 | C5 | E2 |
| 111 | T87 | C14 | C5 | E1 |
| 112 | T88 | C12 | C2 | E3 |

### Comparative Example 1

This comparative example provided Compound DBH1 that had been experimented during research, and the specific structural formula thereof was:

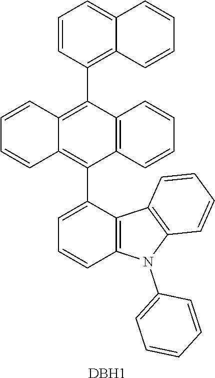 [SMILES: c1ccc(-n2c3ccccc3c3c(-c4c5ccccc5c(-c5cccc6ccccc56)c5ccccc45)cccc32)cc1 ; "DBH1"]

### Comparative Example 2

This comparative example provided Compound DBH2 that had been experimented during research, and the specific structural formula thereof was:

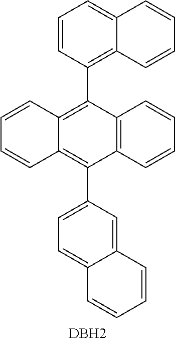

### Comparative Example 3

This comparative example provided Compound BD-1 that had been experimented during research, and the specific structural formula thereof was:

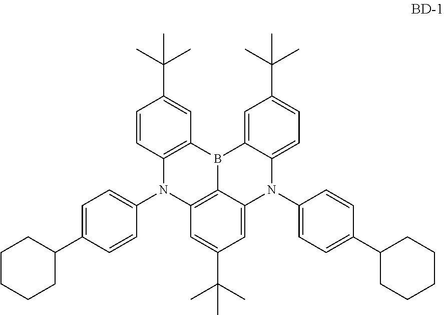 [SMILES: CC(C)(C)c1ccc2c(c1)B1c3cc(C(C)(C)C)ccc3N(c3ccc(C4CCCCC4)cc3)c3cc(C(C)(C)C)cc(c31)N2c1ccc(C2CCCCC2)cc1]

### Comparative Example 4

This comparative example provided Compound BD-2 that had been experimented during research, and the specific structural formula thereof was:

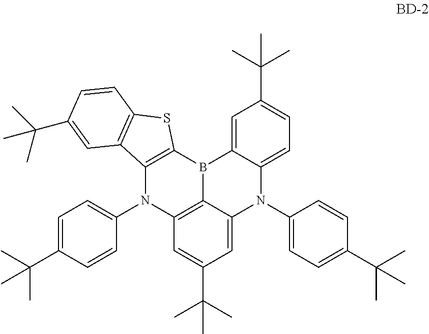 [SMILES: CC(C)(C)c1ccc(N2c3ccc(C(C)(C)C)cc3B3c4sc5ccc(C(C)(C)C)cc5c4N(c4ccc(C(C)(C)C)cc4)c4cc(C(C)(C)C)cc2c43)cc1]

### COMPARATIVE COMPOSITION EXAMPLES

### Comparative Examples 5 to 10

Compositions DT1 to DT6 were obtained by combining the first host compound, the second host compound and a compound as the doping material according to Table 2 and uniformly mixing these materials, wherein the mass ratio of the first host compound to the second host compound to the compound as the doping material in DT1 to DT4 was 49:49:2, and the mass ratio of the host compound to the compound as the doping material in DT5 to DT6 was 98:2.

TABLE 2

| Comparative | | First host | Second host | Doping |
|---|---|---|---|---|
| Example | Composition | compound | compound | material |
| 5 | DT1 | C11 | DBH2 | E10 |
| 6 | DT2 | DBH1 | C4 | E10 |
| 7 | DT3 | C11 | C4 | DBD1 |
| 8 | DT4 | C11 | C4 | DBD2 |
| 9 | DT5 | C11 | — | E10 |
| 10 | DT6 | — | C4 | E10 |

### Evaluation of Performance of Composition

(1) In order to explain the fact that the mixed host material composed of the first host compound and the second host compound in the composition provided by the present invention can balance the carrier transport, the hole mobility uh and electron mobility μe of the mixture composed of the first host compound and the second host compound were calculated respectively, and μe/μh was then calculated. The closer to 1 the value of μe/μh, the more balanced the carrier transport in the mixture composed of the first host compound and the second host compound.

TOF method is currently the most effective method to measure the carrier mobility of an organic semiconductor material. The method can not only test the relatively low mobility of organic small molecules and polymer materials that can be made into a thin film, but also test the mobility of crystal materials that have a relatively low mobility. A time-of-flight (TOF) experiment of carrier mobility was based on the photoconductivity of an organic semiconductor material, and the carrier mobility of the material was calculated by measuring the time it took for the photo-generated carriers in the sample to pass through the sample under an applied electric field and then using the relationship between the mobility and the applied electric field and the carrier drift velocity. The applied deflection voltage was V, the time it took for holes to pass through the thin film device was t, the thickness of the thin film was d, and the mobility u could be expressed as μ=d²/Vt.

The specific experimental steps were as follows: 1) the first host compounds provided by Examples 1 to 6 of the present invention and Comparative Example 1 and the second host compounds provided by Examples 7 to 14 and Comparative Example 2 were uniformly mixed according to Table 3 to obtain mixtures, wherein the mass ratio of the first host compound to the second host compound was 1:1;
\textcircled{2} the mixture was evaporated on ITO to form a 1000 nm thin film, and A1 was then evaporated on the thin film with a thickness of 150 nm to form a cathode, so as to prepare a sample to be tested; and
\textcircled{3} a deflection voltage of 20 volts was applied to the sample to be tested, the hole mobility μh and electron mobility μe of the material were calculated respectively by measuring the time it took for the photo-generated carriers in the sample to be tested to pass through the sample under an applied electric field, and μe/μh was further calculated. The specific calculation results were as shown in Table 3.

TABLE 3

| | First host | Second host | |
|---|---|---|---|
| Mixture | compound | compound | μe/μh |
| 1 | C13 | C5 | 0.975 |
| 2 | C14 | C3 | 0.964 |
| 3 | C12 | C3 | 0.944 |
| 4 | C12 | C2 | 0.942 |
| 5 | C10 | C3 | 1.066 |
| 6 | C14 | C7 | 1.067 |
| 7 | C14 | C1 | 0.905 |
| 8 | C9 | C1 | 1.120 |
| 9 | C14 | C2 | 1.122 |
| 10 | C12 | C5 | 1.129 |
| 11 | C11 | C4 | 0.87 |
| 12 | C10 | C7 | 1.146 |
| 13 | C11 | C6 | 1.151 |
| 14 | C10 | C4 | 1.161 |
| 15 | C12 | C4 | 0.825 |
| 16 | C13 | C7 | 0.762 |
| 17 | C13 | C4 | 0.755 |
| 18 | C12 | C1 | 0.748 |
| 19 | C13 | C2 | 0.737 |
| 20 | C9 | C2 | 1.275 |
| 21 | C9 | C4 | 1.286 |
| 22 | C13 | C8 | 1.366 |
| 23 | C14 | C6 | 1.371 |
| 24 | C9 | C7 | 1.372 |
| 25 | C14 | C5 | 1.384 |
| 26 | C13 | C4 | 0.599 |
| 27 | C10 | C5 | 1.365 |
| 28 | C9 | C6 | 1.372 |
| 29 | C11 | C8 | 1.381 |
| 30 | C12 | C8 | 1.391 |
| 31 | C14 | C8 | 1.395 |
| 32 | C10 | C8 | 1.396 |
| 33 | C09 | C8 | 1.416 |
| 34 | C13 | DBH2 | 0.447 |
| 35 | DBH1 | C5 | 0.523 |
| 36 | DBH1 | DBH2 | 0.297 |

It could be seen from the data in Table 3 that the μe/μh value of the mixed host material composed of the first host compound and the second host compound provided by the present invention was closer to 1, and if either one of the first host compound and the second host compound in the mixed host material provided by the present invention was replaced by another commonly used compound of the same type, the μe/μh value thereof would significantly deviate from 1, indicating that the mixed host material provided by the present invention could effectively balance the carrier transport. The composition provided by the present invention comprised a first host compound containing a pyridylcarbazole structure and a second host compound containing a dibenzofuran structure. From the data in Table 3, it could be seen that the μe/μh value of the mixture composed of C13 and C5 provided in the present application was significantly greater than that of the mixture composed of DBH1 and C5 in the comparative example and was closer to 1. In the present invention, introducing pyridyl into the carbazole and then connecting the pyridylcarbazole fragment to the anthracene fragment can not only enhance the electron-pushing ability of the compound but also adjust the electron mobility of molecules. Compared with the anthracene-based compound (DBH1) containing only a carbazole group, the mixture formed by mixing the compound containing a pyridylcarbazole structure and the compound containing a furan structure provided by the present invention was more suitable for the transport of electrons and holes and could significantly balance the carrier transport.

(2) In order to explain the fact that the composition provided by the present invention was more conducive to the horizontal distribution of the doping material in the host material, the alignment factor of the composition provided by the present invention was tested by means of Hamamatsu Photonics C14234-11 molecular orientation characteristic measurement system. When the alignment factor was equal to 0.67, it was meant that the doping material presented isotropic distribution in the host material, and when the alignment factor was equal to 1, it was meant that the doping material was fully horizontally distributed.

In an organic electroluminescent device, the closer to 1 the alignment factor, the better the horizontal distribution of the molecules of the doping material in the host material, and the more the light emitted from the doping material tends to be emitted in the direction perpendicular to the substrate. By increasing the probability that the light emitted from the doping material was emitted in the direction perpendicular to the substrate, the energy loss caused during light transmission in the organic electroluminescent device could be reduced.

The alignment factors of the compositions provided in Examples 25 to 112 and Comparative Examples 5 to 10 were separately tested by means of Hamamatsu Photonics C14234-11 molecular orientation characteristic measurement system.

The specific experimental steps were as follows: (1) the composition was evaporated on a substrate made of a quartz material; (2) the system excited the test sample by means of an LED light source, causing the test sample to emit light, and the p polarized light in the emitted light was detected by polarizer and received by a detector; and (3) the alignment factor was read by means of an instrument. The specific results were as shown in Table 4.

TABLE 4

| | First host | Second host | Doping | Orientation |
|---|---|---|---|---|
| Composition | compound | compound | material | factor |
| T1 | C11 | C4 | E10 | 0.94 |
| T2 | C12 | C8 | E7 | 0.95 |
| T3 | C13 | C4 | E9 | 0.94 |
| T4 | C12 | C1 | E8 | 0.97 |
| T5 | C11 | C6 | E4 | 0.96 |
| T6 | C13 | C7 | E9 | 0.92 |
| T7 | C9 | C7 | E5 | 0.94 |
| T8 | C10 | C7 | E10 | 0.94 |
| T9 | C12 | C1 | E3 | 0.94 |
| T10 | C10 | C8 | E1 | 0.94 |
| T11 | C14 | C3 | E7 | 0.93 |
| T12 | C10 | C7 | E9 | 0.93 |
| T13 | C10 | C8 | E5 | 0.93 |
| T14 | C9 | C6 | E2 | 0.96 |
| T15 | C14 | C3 | E4 | 0.93 |
| T16 | C12 | C4 | E8 | 0.94 |
| T17 | C13 | C8 | E9 | 0.93 |
| T18 | C10 | C8 | E7 | 0.91 |
| T19 | C9 | C1 | E3 | 0.91 |
| T20 | C14 | C5 | E2 | 0.96 |
| T21 | C14 | C6 | E8 | 0.93 |
| T22 | C12 | C2 | E8 | 0.96 |
| T23 | C14 | C5 | E7 | 0.9 |
| T24 | C14 | C5 | E10 | 0.92 |
| T25 | C13 | C7 | E6 | 0.93 |
| T26 | C9 | C7 | E10 | 0.93 |
| T27 | C11 | C6 | E7 | 0.96 |
| T28 | C13 | C4 | E4 | 0.92 |
| T29 | C9 | C4 | E2 | 0.91 |
| T30 | C12 | C4 | E7 | 0.91 |
| T31 | C12 | C5 | E1 | 0.95 |
| T32 | C9 | C8 | E7 | 0.95 |
| T33 | C9 | C4 | E9 | 0.94 |
| T34 | C14 | C2 | E9 | 0.9 |
| T35 | C12 | C3 | E4 | 0.95 |
| T36 | C13 | C7 | E2 | 0.96 |
| T37 | C12 | C5 | E7 | 0.93 |
| T38 | C14 | C3 | E3 | 0.92 |
| T39 | C12 | C4 | E6 | 0.92 |
| T40 | C13 | C8 | E5 | 0.95 |
| T41 | C9 | C2 | E3 | 0.96 |
| T42 | C14 | C8 | E10 | 0.91 |
| T43 | C11 | C4 | E7 | 0.94 |
| T44 | C14 | C3 | E6 | 0.9 |
| T45 | C10 | C8 | E9 | 0.94 |
| T46 | C12 | C5 | E3 | 0.96 |
| T47 | C9 | C7 | E3 | 0.93 |
| T48 | C9 | C6 | E8 | 0.92 |
| T49 | C12 | C8 | E2 | 0.92 |
| T50 | C9 | C8 | E3 | 0.94 |
| T51 | C14 | C3 | E5 | 0.93 |
| T52 | C9 | C1 | E9 | 0.97 |
| T53 | C13 | C5 | E10 | 0.95 |
| T54 | C12 | C1 | E6 | 0.95 |
| T55 | C9 | C6 | E1 | 0.91 |
| T56 | C13 | C2 | E9 | 0.96 |
| T57 | C9 | C4 | E10 | 0.93 |
| T58 | C14 | C6 | E2 | 0.9 |
| T59 | C11 | C4 | E8 | 0.95 |
| T60 | C14 | C2 | E2 | 0.94 |
| T61 | C14 | C7 | E1 | 0.95 |
| T62 | C9 | C7 | E9 | 0.92 |
| T63 | C10 | C3 | E2 | 0.91 |
| T64 | C14 | C2 | E1 | 0.91 |
| T65 | C12 | C8 | E4 | 0.94 |
| T66 | C14 | C8 | E7 | 0.92 |
| T67 | C14 | C5 | E5 | 0.95 |
| T68 | C12 | C3 | E10 | 0.95 |
| T69 | C14 | C8 | E3 | 0.94 |
| T70 | C9 | C7 | E7 | 0.9 |
| T71 | C9 | C7 | E2 | 0.94 |
| T72 | C9 | C7 | E6 | 0.9 |
| T73 | C10 | C4 | E9 | 0.94 |
| T74 | C11 | C8 | E5 | 0.92 |
| T75 | C14 | C7 | E9 | 0.94 |
| T76 | C12 | C8 | E8 | 0.92 |
| T77 | C9 | C2 | E7 | 0.91 |
| T78 | C12 | C1 | E5 | 0.92 |
| T79 | C9 | C1 | E10 | 0.93 |
| T80 | C13 | C5 | E6 | 0.91 |
| T81 | C10 | C4 | E5 | 0.9 |
| T82 | C11 | C4 | E4 | 0.96 |
| T83 | C9 | C6 | E7 | 0.95 |
| T84 | C13 | C4 | E8 | 0.93 |
| T85 | C13 | C5 | E3 | 0.91 |
| T86 | C13 | C5 | E2 | 0.92 |
| T87 | C14 | C5 | E1 | 0.93 |
| T88 | C12 | C2 | E3 | 0.95 |
| DT1 | C11 | DBH2 | E10 | 0.88 |
| DT2 | DBH1 | C4 | E10 | 0.89 |
| DT3 | C11 | C4 | DBD1 | 0.86 |
| DT4 | C11 | C4 | DBD2 | 0.86 |
| DT5 | C11 | — | E10 | 0.81 |
| DT6 | — | C4 | E10 | 0.83 |

From the data in Table 4, it could be seen that compared with the compositions of the comparative examples, the alignment factor of the composition provided by the present invention was closer to 1. If any one of the compound as the doping material, the first host compound, or the second host compound in the composition T1 provided by the present invention was replaced with another commonly used compound of the same type or removed, the alignment factor thereof would significantly deviate from 1, indicating that the doping material provided by the present invention and the mixed host material provided by the present invention had good alignment, and the molecules of the doping material provided by the present invention could be horizontally distributed in the mixed host material provided by the present invention. According to the test results, it could be seen that the alignment factor of the composition was not only affected by the structure of the doping material, but also by the structure of the host material. In the composition provided by the present invention, by separately defining the structures of the doping material, the first host compound, and the second host compound and by means of the interaction between the doping material and the first and second host compounds, the molecules of the doping material in the composition can be horizontally distributed in the mixed host composed of the first host compound and the second host compound, the light emitted from the doping material tends to be emitted in the direction perpendicular to the substrate, thereby reducing the energy loss caused during light transmission in the organic electroluminescent device.

### Device Example 1

This example provided a blue-light organic electroluminescent device, the preparation method of which was as follows: firstly, on an ITO layer (anode) formed on a substrate, HTL and p-dopant (at a mass ratio of HTL to p-dopant of 97:3) were deposited in vacuo to a thickness of 10 nm to form a hole injection layer; secondly, on the above hole injection layer, HTL was deposited in vacuo to a thickness of 120 nm to form a hole transport layer; thirdly, on the above hole transport layer, B prime was deposited in vacuo to a thickness of 5 nm to form a luminescent auxiliary layer; again, on the above luminescent auxiliary layer, a mixture of a host material and a doping material was vacuum-deposited with a thickness of 20 nm to form a luminescent layer, wherein C11 and C4 jointly acted as a host material, C11 was the first host compound, C4 was the second host compound, and E10 was the doping material, and the mass ratio of the first host compound to the second host compound to the doping material was 49:49:2; next, on the above luminescent layer, HBL was deposited in vacuo to a thickness of 5 nm to form a hole barrier layer; a mixture of ET and Liq (the mass ratio of ET to Liq was 1:1) was deposited in vacuo to a thickness of 30 nm to form an electron transport layer; then, on the above electron transport layer, LiF was deposited to a thickness of 0.2 nm to form an electron injection layer; and finally, on the above electron injection layer, aluminum (Al) was deposited to a thickness of 150 nm to form a cathode, thereby preparing a blue-light organic electroluminescent device.

The molecular structural formulas of the materials of the layers other than the host materials and the doping material in the luminescent layer were as follows:

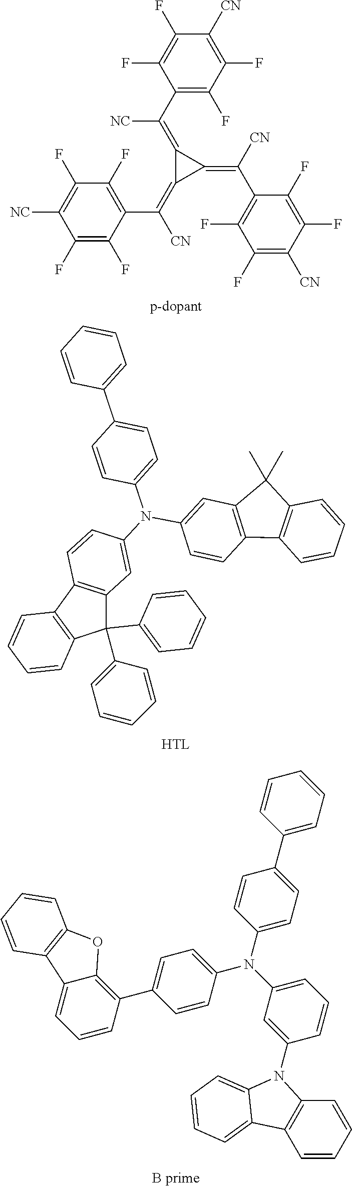 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; CC1(C)c2ccccc2-c2ccc(N(c3ccc(-c4ccccc4)cc3)c3ccc4c(c3)C(c3ccccc3)(c3ccccc3)c3ccccc3-4)cc21 ; c1ccc(-c2ccc(N(c3ccc(-c4cccc5c4oc4ccccc45)cc3)c3cccc(-n4c5ccccc5c5ccccc54)c3)cc2)cc1]

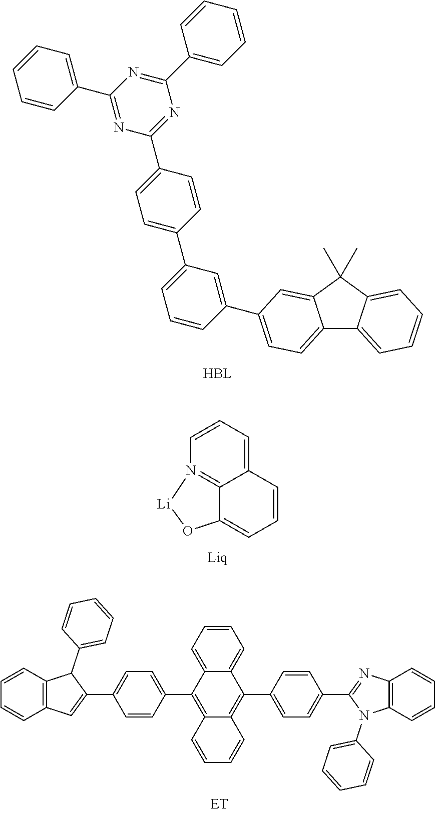

The electrode preparation method and the deposition method for each functional layer in this implementation were both conventional methods in the art, such as vacuum thermal evaporation or ink-jet printing. No more unnecessary repetition would be given here.

### Device Examples 2-100

The method was the same as that in Device Example 1, except that the first host compound, the second host compound, and the doping material were combined according to Table 5.

TABLE 5
Comparison table of host materials and
doping materials in device examples

| | First host | Second host | Doping |
|---|---|---|---|
| Device Example | compound | compound | material |
| Device Example 2 | C12 | C8 | E7 |
| Device Example 3 | C13 | C4 | E9 |
| Device Example 4 | C12 | C1 | E8 |
| Device Example 5 | C11 | C6 | E4 |
| Device Example 6 | C13 | C7 | E9 |
| Device Example 7 | C9 | C7 | E5 |
| Device Example 8 | C10 | C7 | E10 |
| Device Example 9 | C12 | C1 | E3 |
| Device Example 10 | C10 | C8 | E1 |
| Device Example 11 | C14 | C3 | E7 |
| Device Example 12 | C10 | C7 | E9 |
| Device Example 13 | C10 | C8 | E5 |
| Device Example 14 | C9 | C6 | E2 |
| Device Example 15 | C14 | C3 | E4 |
| Device Example 16 | C12 | C4 | E8 |
| Device Example 17 | C13 | C8 | E9 |
| Device Example 18 | C10 | C8 | E7 |
| Device Example 19 | C9 | C1 | E3 |
| Device Example 20 | C14 | C5 | E2 |
| Device Example 21 | C14 | C6 | E8 |
| Device Example 22 | C12 | C2 | E8 |
| Device Example 23 | C14 | C5 | E7 |
| Device Example 24 | C14 | C5 | E10 |
| Device Example 25 | C13 | C7 | E6 |
| Device Example 26 | C9 | C7 | E10 |
| Device Example 27 | C11 | C6 | E7 |
| Device Example 28 | C14 | C6 | E8 |
| Device Example 29 | C13 | C4 | E4 |
| Device Example 30 | C9 | C4 | E2 |
| Device Example 31 | C12 | C4 | E7 |
| Device Example 32 | C12 | C5 | E1 |
| Device Example 33 | C12 | C2 | E8 |
| Device Example 34 | C9 | C8 | E7 |
| Device Example 35 | C9 | C4 | E9 |
| Device Example 36 | C14 | C2 | E9 |
| Device Example 37 | C12 | C3 | E4 |
| Device Example 38 | C13 | C7 | E2 |
| Device Example 39 | C12 | C5 | E7 |
| Device Example 40 | C14 | C3 | E3 |
| Device Example 41 | C12 | C4 | E6 |
| Device Example 42 | C13 | C8 | E5 |
| Device Example 43 | C9 | C2 | E3 |
| Device Example 44 | C14 | C8 | E10 |
| Device Example 45 | C11 | C4 | E7 |
| Device Example 46 | C14 | C3 | E6 |
| Device Example 47 | C13 | C8 | E9 |
| Device Example 48 | C10 | C8 | E9 |
| Device Example 49 | C12 | C5 | E3 |
| Device Example 50 | C9 | C7 | E3 |
| Device Example 51 | C14 | C3 | E7 |
| Device Example 52 | C12 | C5 | E3 |
| Device Example 53 | C9 | C6 | E8 |
| Device Example 54 | C12 | C8 | E2 |
| Device Example 55 | C14 | C3 | E3 |
| Device Example 56 | C9 | C8 | E3 |
| Device Example 57 | C14 | C3 | E5 |
| Device Example 58 | C9 | C1 | E9 |
| Device Example 59 | C13 | C5 | E10 |
| Device Example 60 | C12 | C1 | E6 |
| Device Example 61 | C9 | C6 | E1 |
| Device Example 62 | C14 | C3 | E5 |
| Device Example 63 | C13 | C2 | E9 |
| Device Example 64 | C9 | C4 | E10 |
| Device Example 65 | C13 | C4 | E9 |
| Device Example 66 | C14 | C6 | E2 |
| Device Example 67 | C11 | C4 | E8 |
| Device Example 68 | C14 | C2 | E2 |
| Device Example 69 | C14 | C7 | E1 |
| Device Example 70 | C9 | C7 | E9 |
| Device Example 71 | C10 | C3 | E2 |
| Device Example 72 | C14 | C2 | E1 |
| Device Example 73 | C12 | C8 | E4 |
| Device Example 74 | C14 | C8 | E7 |
| Device Example 75 | C14 | C5 | E5 |
| Device Example 76 | C12 | C3 | E10 |
| Device Example 77 | C14 | C8 | E3 |
| Device Example 78 | C10 | C7 | E10 |
| Device Example 79 | C9 | C7 | E7 |
| Device Example 80 | C14 | C2 | E2 |
| Device Example 81 | C9 | C7 | E2 |
| Device Example 82 | C9 | C7 | E6 |
| Device Example 83 | C10 | C4 | E9 |
| Device Example 84 | C11 | C8 | E5 |
| Device Example 85 | C14 | C7 | E9 |
| Device Example 86 | C12 | C8 | E8 |
| Device Example 87 | C9 | C2 | E7 |
| Device Example 88 | C12 | C1 | E5 |
| Device Example 89 | C9 | C1 | E10 |
| Device Example 90 | C13 | C5 | E6 |
| Device Example 91 | C10 | C4 | E5 |
| Device Example 92 | C11 | C4 | E4 |
| Device Example 93 | C10 | C3 | E2 |
| Device Example 94 | C13 | C7 | E2 |
| Device Example 95 | C9 | C6 | E7 |
| Device Example 96 | C13 | C4 | E8 |
| Device Example 97 | C13 | C5 | E3 |
| Device Example 98 | C13 | C5 | E2 |
| Device Example 99 | C14 | C5 | E1 |
| Device Example 100 | C12 | C2 | E3 |

### Comparative Device Examples 1-6

The method was the same as that in Device Example 1, except that the luminescent layer material was replaced with the compositions of Comparative Examples 5 to 10. The host materials and doping materials in the comparative device examples were found in Table 6.

TABLE 6
Comparison table of host materials and doping
materials in comparative device examples

| Comparative Device | First host | Second host | Doping |
|---|---|---|---|
| Example | compound | compound | material |
| Comparative Device | C11 | DBH2 | E10 |
| Example 1 | | | |
| Comparative Device | DBH1 | C4 | E10 |
| Example 2 | | | |
| Comparative Device | C11 | C4 | DBD1 |
| Example 3 | | | |
| Comparative Device | C11 | C4 | DBD2 |
| Example 4 | | | |
| Comparative Device | C11 | — | E10 |
| Example 5 | | | |
| Comparative Device | — | C4 | E10 |
| Example 6 | | | |

### Device Performance Effect Example

The organic electroluminescent devices provided by Device Examples 1-100 and Comparative Device Examples 1-6 were tested by standard methods. In this regard, the organic electroluminescent devices were measured at a current density of J=10 mA/cm² for the driving voltage, brightness, electroluminescent current efficiency (measured as cd/A), and external quantum efficiency (EQE, measured by percentage), calculated, as a function of luminous density, from the current/voltage/luminous density characteristic curves (IVL characteristic curves) showing Lambertian emission characteristics, luminous spectrum. The lifetime LT was defined as the time for the brightness to decrease from the initial luminous brightness L₀ to a specific proportion L₁, during working at a constant current J; The expressions J=50 mA/cm² and L₁=90% meant that during working at 50 mA/cm², the luminous brightness decreased to 90% of the initial value L₀ thereof after the time LT. Similarly, the expressions J=20 mA/cm² and L₁=80% meant that during working at 20 mA/cm², the luminous brightness decreased to 80% of the initial value L₀ thereof after the time LT.

The test instruments and methods for testing the performance of the above OLED devices were as follows:
the brightness was tested by means of spectrum scanner PhotoResearch PR-635;
the current density and turn-on voltage were tested by digital SourceMeter Keithley 2400; and
lifetime test: LT-96ch lifetime test device was used.

The performance test results of the above devices were listed in Table 7.

TABLE 7
Performance test results of blue-light devices

| | | | @J = 20 | |
|---|---|---|---|---|
| | Vop | | mA/cm² | |
| | (V) | EQE(%) | LT95 (h) | Color |
| Device Example 1 | 3.78 | 8.34 | 159 | Blue |
| Device Example 2 | 3.81 | 7.72 | 135 | Blue |
| Device Example 3 | 3.79 | 8.23 | 156 | Blue |
| Device Example 4 | 3.74 | 8.23 | 150 | Blue |
| Device Example 5 | 3.73 | 8.32 | 158 | Blue |
| Device Example 6 | 3.8 | 8.22 | 152 | Blue |
| Device Example 7 | 3.76 | 8.11 | 147 | Blue |
| Device Example 8 | 3.82 | 8.3 | 161 | Blue |
| Device Example 9 | 3.8 | 8.19 | 156 | Blue |
| Device Example 10 | 3.82 | 7.25 | 114 | Blue |
| Device Example 11 | 3.72 | 8.41 | 160 | Blue |
| Device Example 12 | 3.77 | 8.33 | 158 | Blue |
| Device Example 13 | 3.77 | 7.21 | 116 | Blue |
| Device Example 14 | 3.71 | 7.89 | 138 | Blue |
| Device Example 15 | 3.71 | 8.43 | 161 | Blue |
| Device Example 16 | 3.8 | 8.29 | 159 | Blue |
| Device Example 17 | 3.82 | 8.07 | 146 | Blue |
| Device Example 18 | 3.72 | 7.19 | 112 | Blue |
| Device Example 19 | 3.76 | 8.29 | 158 | Blue |
| Device Example 20 | 3.83 | 8.39 | 160 | Blue |
| Device Example 21 | 3.85 | 8.09 | 151 | Blue |
| Device Example 22 | 3.75 | 8.45 | 158 | Blue |
| Device Example 23 | 3.75 | 8.04 | 148 | Blue |
| Device Example 24 | 3.77 | 8.05 | 149 | Blue |
| Device Example 25 | 3.85 | 8.21 | 154 | Blue |
| Device Example 26 | 3.71 | 8.1 | 150 | Blue |
| Device Example 27 | 3.83 | 8.32 | 158 | Blue |
| Device Example 28 | 3.74 | 8.07 | 150 | Blue |
| Device Example 29 | 3.72 | 8.22 | 157 | Blue |
| Device Example 30 | 3.78 | 8.16 | 154 | Blue |
| Device Example 31 | 3.83 | 8.26 | 154 | Blue |
| Device Example 32 | 3.72 | 8.35 | 158 | Blue |
| Device Example 33 | 3.83 | 8.39 | 165 | Blue |
| Device Example 34 | 3.71 | 7.01 | 108 | Blue |
| Device Example 35 | 3.82 | 8.18 | 154 | Blue |
| Device Example 36 | 3.73 | 8.31 | 162 | Blue |
| Device Example 37 | 3.78 | 8.4 | 162 | Blue |
| Device Example 38 | 3.77 | 8.25 | 157 | Blue |
| Device Example 39 | 3.7 | 8.34 | 158 | Blue |
| Device Example 40 | 3.83 | 8.42 | 166 | Blue |
| Device Example 41 | 3.83 | 8.28 | 160 | Blue |
| Device Example 42 | 3.81 | 8.13 | 153 | Blue |
| Device Example 43 | 3.76 | 8.19 | 156 | Blue |
| Device Example 44 | 3.82 | 7.48 | 125 | Blue |
| Device Example 45 | 3.81 | 8.33 | 162 | Blue |
| Device Example 46 | 3.83 | 8.41 | 163 | Blue |
| Device Example 47 | 3.72 | 8.1 | 147 | Blue |
| Device Example 48 | 3.73 | 7.22 | 116 | Blue |
| Device Example 49 | 3.7 | 8.38 | 157 | Blue |
| Device Example 50 | 3.84 | 8.07 | 147 | Blue |
| Device Example 51 | 3.81 | 8.38 | 162 | Blue |
| Device Example 52 | 3.81 | 8.34 | 155 | Blue |
| Device Example 53 | 3.78 | 7.87 | 141 | Blue |
| Device Example 54 | 3.83 | 7.69 | 135 | Blue |
| Device Example 55 | 3.84 | 8.39 | 165 | Blue |
| Device Example 56 | 3.79 | 7.02 | 106 | Blue |
| Device Example 57 | 3.75 | 8.43 | 161 | Blue |
| Device Example 58 | 3.72 | 8.38 | 163 | Blue |
| Device Example 59 | 3.84 | 8.46 | 159 | Blue |
| Device Example 60 | 3.75 | 8.23 | 150 | Blue |
| Device Example 61 | 3.83 | 7.83 | 144 | Blue |
| Device Example 62 | 3.83 | 8.41 | 160 | Blue |
| Device Example 63 | 3.77 | 8.22 | 152 | Blue |
| Device Example 64 | 3.8 | 8.18 | 154 | Blue |
| Device Example 65 | 3.72 | 8.16 | 151 | Blue |
| Device Example 66 | 3.8 | 8.07 | 153 | Blue |
| Device Example 67 | 3.72 | 8.35 | 155 | Blue |
| Device Example 68 | 3.82 | 8.37 | 156 | Blue |
| Device Example 69 | 3.76 | 8.4 | 160 | Blue |
| Device Example 70 | 3.7 | 8.08 | 151 | Blue |
| Device Example 71 | 3.84 | 8.36 | 163 | Blue |
| Device Example 72 | 3.79 | 8.3 | 158 | Blue |
| Device Example 73 | 3.83 | 7.72 | 138 | Blue |
| Device Example 74 | 3.72 | 7.45 | 126 | Blue |
| Device Example 75 | 3.81 | 8.1 | 152 | Blue |
| Device Example 76 | 3.75 | 8.4 | 161 | Blue |
| Device Example 77 | 3.74 | 7.48 | 123 | Blue |
| Device Example 78 | 3.77 | 8.33 | 154 | Blue |
| Device Example 79 | 3.77 | 8.06 | 146 | Blue |
| Device Example 80 | 3.76 | 8.35 | 157 | Blue |
| Device Example 81 | 3.75 | 8.11 | 149 | Blue |
| Device Example 82 | 3.8 | 8.07 | 149 | Blue |
| Device Example 83 | 3.83 | 8.3 | 158 | Blue |
| Device Example 84 | 3.78 | 7.76 | 140 | Blue |
| Device Example 85 | 3.74 | 8.39 | 158 | Blue |
| Device Example 86 | 3.77 | 7.71 | 134 | Blue |
| Device Example 87 | 3.79 | 8.14 | 156 | Blue |
| Device Example 88 | 3.82 | 8.18 | 154 | Blue |
| Device Example 89 | 3.84 | 8.35 | 156 | Blue |
| Device Example 90 | 3.78 | 8.39 | 166 | Blue |
| Device Example 91 | 3.71 | 8.29 | 157 | Blue |
| Device Example 92 | 3.78 | 8.37 | 158 | Blue |
| Device Example 93 | 3.77 | 8.39 | 162 | Blue |
| Device Example 94 | 3.77 | 8.2 | 154 | Blue |
| Device Example 95 | 3.75 | 7.87 | 142 | Blue |
| Device Example 96 | 3.72 | 8.19 | 154 | Blue |
| Device Example 97 | 3.75 | 8.43 | 163 | Blue |
| Device Example 98 | 3.85 | 8.44 | 160 | Blue |
| Device Example 99 | 3.78 | 8.08 | 149 | Blue |
| Device Example 100 | 3.74 | 8.44 | 165 | Blue |
| Comparative Device | 4.32 | 6.62 | 85 | Blue |
| Example 1 | | | | |
| Comparative Device | 4.22 | 6.76 | 75 | Blue |
| Example 2 | | | | |
| Comparative Device | 3.99 | 6.41 | 106 | Blue |
| Example 3 | | | | |
| Comparative Device | 4.05 | 6.39 | 90 | Blue |
| Example 4 | | | | |
| Comparative Device | 4.61 | 6.13 | 82 | Blue |
| Example 5 | | | | |
| Comparative Device | 4.52 | 6.06 | 76 | Blue |
| Example 6 | | | | |

From the device performance test results in Table 7 above, it could be seen that compared with the devices of the comparative examples, the efficiency and lifetime of the organic electroluminescent device provided by the present invention were significantly improved. If any one of the doping material, the first host compound, or the second host compound in the organic electroluminescent device provided by the present invention was replaced with another commonly used compound of the same type or removed, the efficiency and lifetime thereof would both significantly increase. The reason lay in that in the first host compound provided by the present invention, introducing pyridyl into the carbazole and then connecting the pyridylcarbazole fragment to the anthracene fragment can not only enhance the electron-pushing ability of the compound but also adjust the electron mobility of molecules. Compared with the anthracene-based compound containing only a carbazole group, the mixed host material composed of the first host compound and the second host compound provided in the present invention was more suitable for the transport of electrons and holes and can significantly improve the balance of carriers in the blue light host material, so that the recombination center was located in the center of the luminescent layer, which could avoid the energy loss caused by the deviation of the recombination center, reduced the risk of material decomposition in adjacent layers, and could thus improve the efficiency and lifetime of the organic electroluminescent device. In the present invention, by introducing a pyridine fragment into the carbazole group in the first host compound, which pyridine can form hydrogen bonds with the molecule of the second host compound and the molecule of the doping material, the film formation becomes more stable, and the luminescence lifetime of the organic electroluminescent device can thus be improved. In the composition provided by the present invention, by separately defining the structures of the doping material, the first host compound, and the second host compound and by means of the interaction between the doping material and the first and second host compounds, the molecules of the doping material in the composition can be horizontally distributed in the mixed host composed of the first host compound and the second host compound, the light emitted from the doping material tends to be emitted in the direction perpendicular to the substrate, thereby reducing the energy loss caused during light transmission in the organic electroluminescent device, thus improving the efficiency of the organic electroluminescent device. In the present invention, by means of the rational combination of the double host materials and the doping material, the interaction of the first host compound, the second host compound, and the compound as a doping material imparts the organic electroluminescent device with the significant advantages of a high efficiency and a long lifetime. The use of the composition provided by the present invention as the luminescent layer material could improve the luminous efficiency of a blue organic electroluminescent device, prolong the lifetime of the blue organic electroluminescent device and overcome the defects in the prior art.

The above description is only preferred embodiments of the present invention, and the scope of protection of the present invention is not limited thereto. Any changes, substitutions, etc. readily conceivable to any of those familiar with the technical field within the technical scope of the disclosure of the present invention should be included in the scope of protection of the present invention. Therefore, for the scope of protection of the present invention, the scope of protection of the claims shall prevail.

1. An organic electroluminescent compound, characterized in that the organic electroluminescent compound has a general structural formula as shown in Formula I: 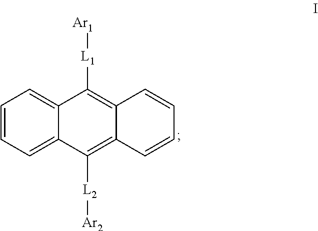
wherein Ar₁ is selected from a substituted or unsubstituted aryl group with a carbon atom number of 6 to 60 and a substituted or unsubstituted fused aryl group with a carbon atom number of 10 to 60;
L₁ and L₂ are each independently selected from a single bond and a substituted or unsubstituted arylene group with a carbon atom number of 6 to 30;
Ar₂ is 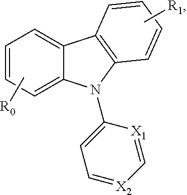
in which X₁ and X₂ are selected from C or N, and R₀ and R₁ respectively represent a single substituent to the maximum allowable number of substituents, and each of the substituents is independently selected from hydrogen, deuterium, a substituted or unsubstituted alkyl group with a carbon atom number of 1 to 10, and a substituted or unsubstituted alkenyl group with a carbon atom number of 2 to 10, wherein two or more of the substituents can be bonded to each other via a linker group or a single bond to form a benzene ring or a fused ring; and
when any one of Ar₁, L₁, L₂, R₀, and R₁ has substituents, Ar₁, L₁, L₂, R₀, and R₁ can have one or more substituents, and each of the substituents is independently selected from deuterium, an aryl group with a carbon atom number of 6 to 30, and a fused cyclic aryl group with a carbon atom number of 10 to 30.

2. The organic electroluminescent compound according to claim 1, characterized in that Ar₂ is selected from any one of

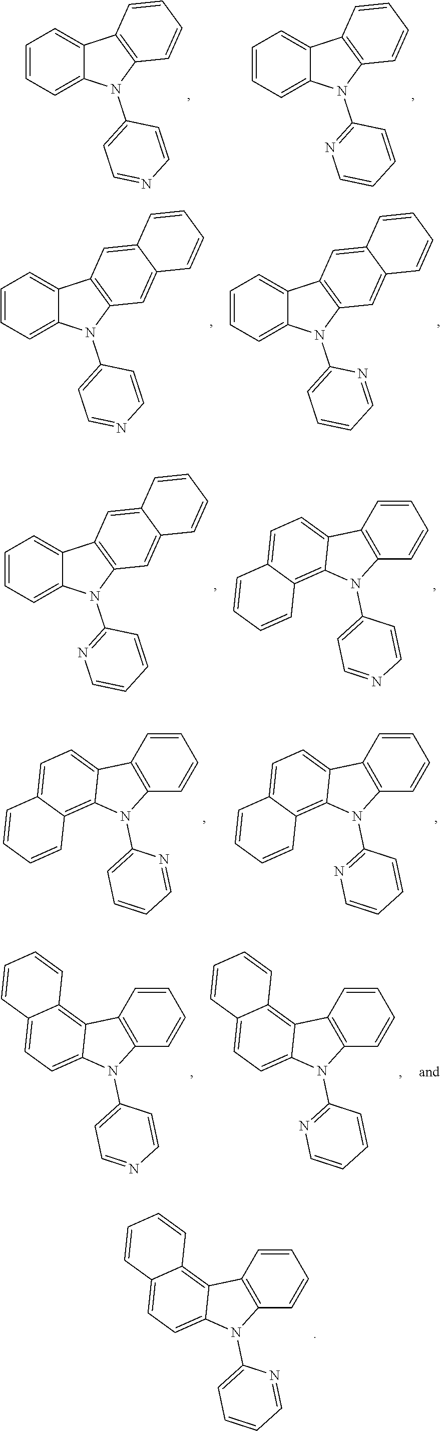

3. The organic electroluminescent compound according to claim 1, characterized in that the organic electroluminescent compound is selected from any one of the following structures:

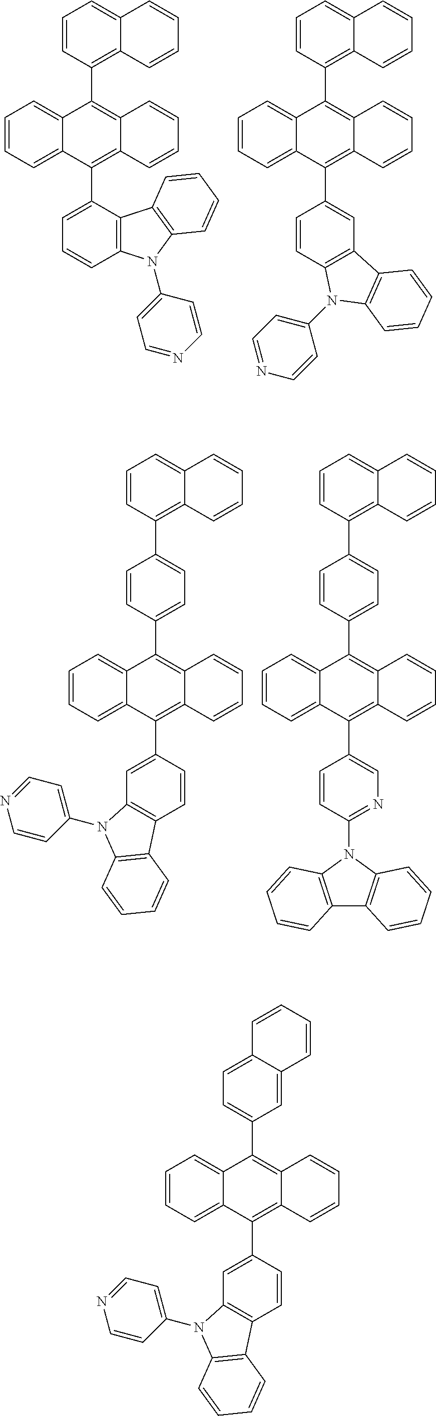

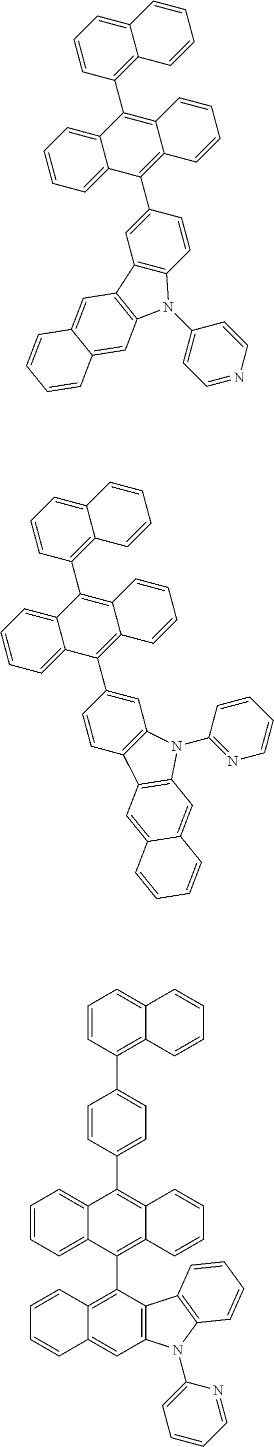 [SMILES: c1ccc2cc3c(cc2c1)c1cc(-c2c4ccccc4c(-c4cccc5ccccc45)c4ccccc24)ccc1n3-c1ccncc1 ; c1ccc(-n2c3cc(-c4c5ccccc5c(-c5cccc6ccccc56)c5ccccc45)ccc3c3cc4ccccc4cc32)nc1 ; c1ccc(-n2c3ccccc3c3c(-c4c5ccccc5c(-c5ccc(-c6cccc7ccccc67)cc5)c5ccccc45)c4ccccc4cc32)nc1]

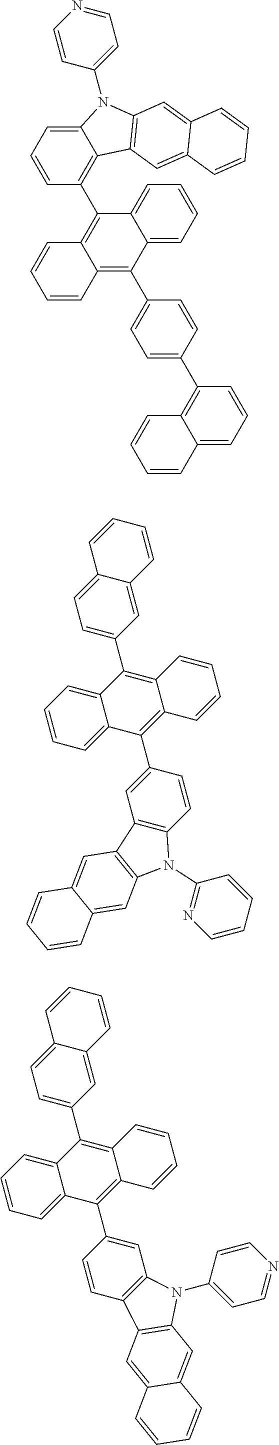

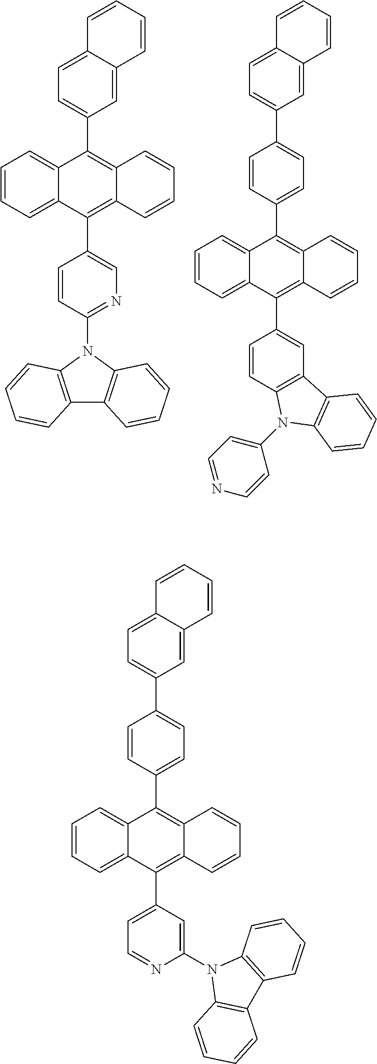 [SMILES: c1ccc2cc(-c3c4ccccc4c(-c4ccc(-n5c6ccccc6c6ccccc65)nc4)c4ccccc34)ccc2c1 ; c1ccc2cc(-c3ccc(-c4c5ccccc5c(-c5ccc6c(c5)c5ccccc5n6-c5ccncc5)c5ccccc45)cc3)ccc2c1 ; c1ccc2cc(-c3ccc(-c4c5ccccc5c(-c5ccnc(-n6c7ccccc7c7ccccc76)c5)c5ccccc45)cc3)ccc2c1]

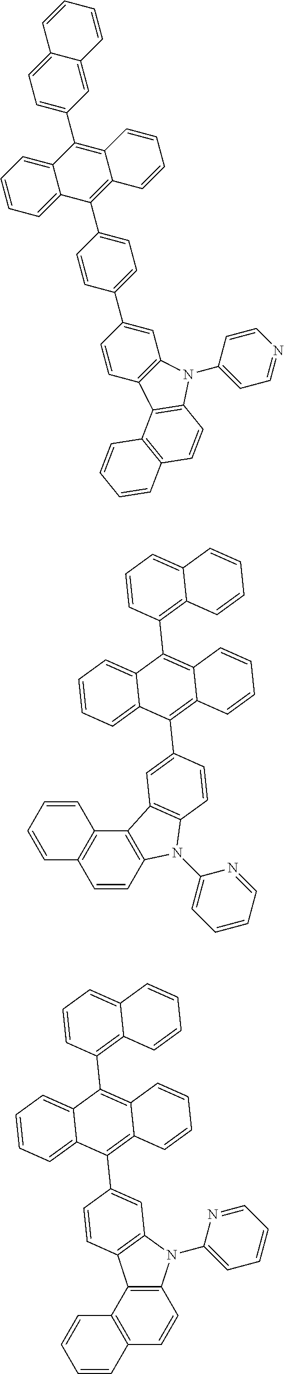 [SMILES: c1ccc2cc(-c3c4ccccc4c(-c4ccc(-c5ccc6c7c8ccccc8ccc7n(-c7ccncc7)c6c5)cc4)c4ccccc34)ccc2c1 ; c1ccc(-n2c3ccc(-c4c5ccccc5c(-c5cccc6ccccc56)c5ccccc45)cc3c3c4ccccc4ccc32)nc1 ; c1ccc(-n2c3cc(-c4c5ccccc5c(-c5cccc6ccccc56)c5ccccc45)ccc3c3c4ccccc4ccc32)nc1]

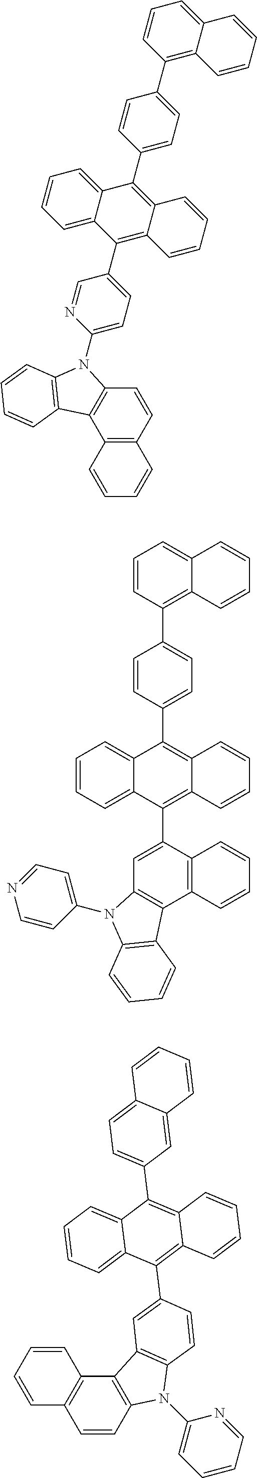 [SMILES: c1ccc2c(-c3ccc(-c4c5ccccc5c(-c5ccc(-n6c7ccccc7c7c8ccccc8ccc76)nc5)c5ccccc45)cc3)cccc2c1 ; c1ccc2c(-c3ccc(-c4c5ccccc5c(-c5cc6c(c7ccccc57)c5ccccc5n6-c5ccncc5)c5ccccc45)cc3)cccc2c1 ; c1ccc(-n2c3ccc(-c4c5ccccc5c(-c5ccc6ccccc6c5)c5ccccc45)cc3c3c4ccccc4ccc32)nc1]

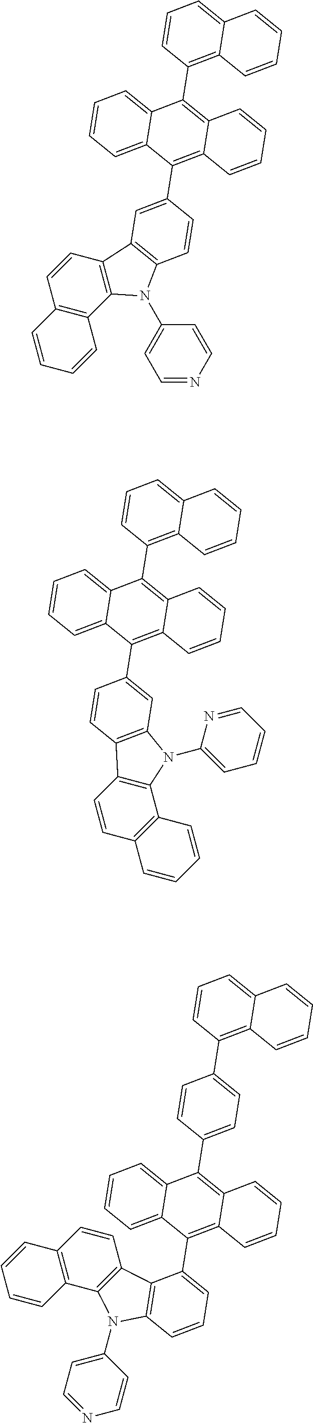 [SMILES: c1ccc2c(-c3c4ccccc4c(-c4ccc5c(c4)c4ccc6ccccc6c4n5-c4ccncc4)c4ccccc34)cccc2c1 ; c1ccc(-n2c3cc(-c4c5ccccc5c(-c5cccc6ccccc56)c5ccccc45)ccc3c3ccc4ccccc4c32)nc1 ; c1ccc2c(-c3ccc(-c4c5ccccc5c(-c5cccc6c5c5ccc7ccccc7c5n6-c5ccncc5)c5ccccc45)cc3)cccc2c1]

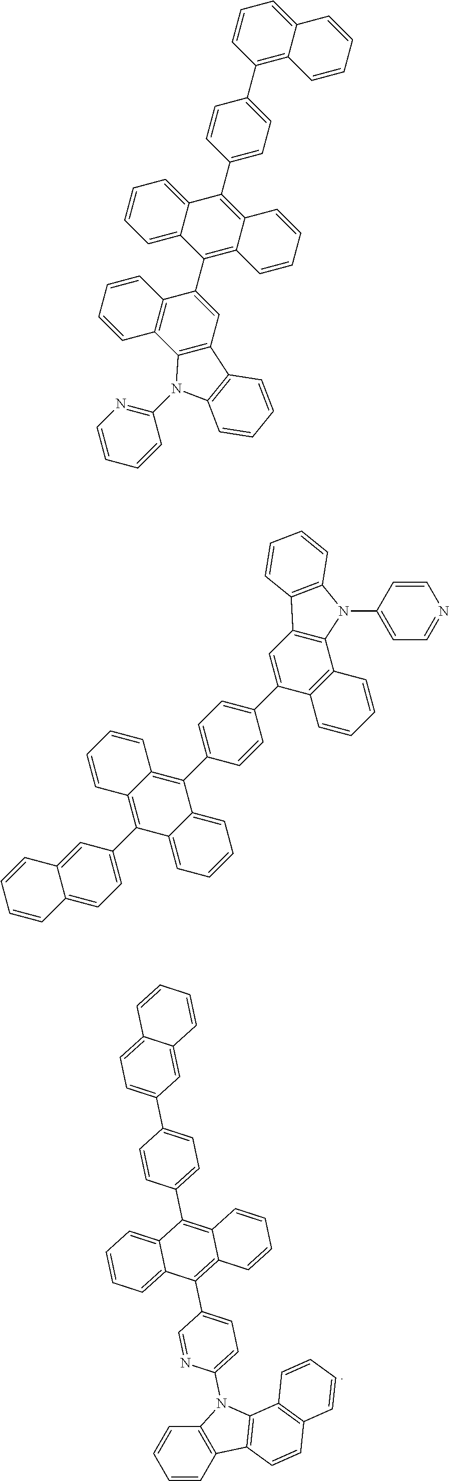 [SMILES: c1ccc(-n2c3ccccc3c3cc(-c4c5ccccc5c(-c5ccc(-c6cccc7ccccc67)cc5)c5ccccc45)c4ccccc4c32)nc1 ; c1ccc2cc(-c3c4ccccc4c(-c4ccc(-c5cc6c7ccccc7n(-c7ccncc7)c6c6ccccc56)cc4)c4ccccc34)ccc2c1 ; c1ccc2cc(-c3ccc(-c4c5ccccc5c(-c5ccc(-n6c7ccccc7c7ccc8ccccc8c76)nc5)c5ccccc45)cc3)ccc2c1]

4. A composition, characterized in that the composition comprises one or more organic electroluminescent compounds according to claim 1, and the composition further comprises one or more compounds represented by Formula II and one or more compounds represented by Formula III: 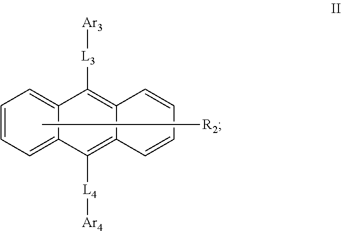
wherein Ar₃ is selected from a substituted or unsubstituted aryl group with a carbon atom number of 6 to 60 and a substituted or unsubstituted fused aryl group with a carbon atom number of 10 to 60;
R₂ represents a single substituent to the maximum allowable number of substituents, and each of the substituents is independently selected from hydrogen or deuterium;
L₃ and L₄ are each independently selected from a single bond and a substituted or unsubstituted arylene group with a carbon atom number of 6 to 30;
Ar₄ is 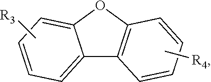
in which R₃ and R₄ represent a single substituent to the maximum allowable number of substituents, and each of the substituents is independently selected from hydrogen, deuterium, a substituted or unsubstituted alkyl group with a carbon atom number of 1 to 10, and a substituted or unsubstituted alkenyl group with a carbon atom number of 2 to 10, wherein two or more of the substituents can be bonded to each other via a linker group or a single bond to form a benzene ring or a fused ring; and
when any one of Ar₃, L₃, L₄, R₃, and R₄ has substituents, Ar₃, L₃, L₄, R₃, and R₄ can have one or more substituents, and each of the substituents is independently selected from deuterium, an aryl group with a carbon atom number of 6 to 30, and a fused cyclic aryl group with a carbon atom number of 10 to 30; and 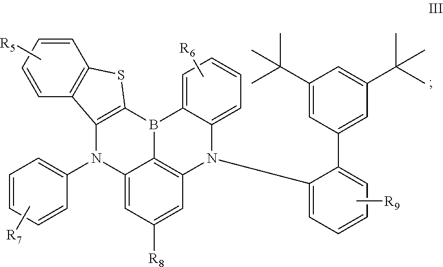
wherein R₅, R₆, and R₈ each independently represent a single substituent to the maximum allowable number of substituents, and each of the substituents is independently selected from any one of or a combination of any two or more of hydrogen, deuterium, a substituted or unsubstituted alkyl group with a carbon atom number of 1 to 10, a substituted or unsubstituted cycloalkyl group with a carbon atom number of 3 to 30, a substituted or unsubstituted aryl group with a carbon atom number of 6 to 30, and a substituted or unsubstituted arylamine group with a carbon atom number of 12 to 30, wherein two or more of the substituents can be bonded to each other via a linker group or a single bond to form an aliphatic ring, an aromatic ring, a heteroaromatic ring, a fused ring, or a fused heterocyclic ring;
R₇ represents a substituted or unsubstituted cycloalkyl group with a carbon atom number of 3 to 30 or a substituted or unsubstituted bridged cycloalkyl group with a carbon atom number of 4 to 30; and when R₇ has substituents, R₇ can have one or more substituents, each of which is independently selected from an alkyl group with a carbon atom number of 1 to 10 or an aryl group with a carbon atom number of 6 to 30, and two or more of the substituents can be bonded to each other via a linker group or a single bond to form an aliphatic ring;
R₉ represents a single substituent to the maximum allowable number of substituents, and each of the substituents is independently selected from any one of or a combination of any two or more of hydrogen, deuterium, a substituted or unsubstituted alkyl group with a carbon atom number of 1 to 10, and a substituted or unsubstituted aryl group with a carbon atom number of 6 to 30, wherein two or more of the substituents can be bonded to each other via a linker group or a single bond to form an aliphatic ring, an aromatic ring, a heteroaromatic ring, a fused ring, or a fused heterocyclic ring; and
when any one of R₅, R₆, R₈, and R₉ has substituents, R₅, R₆, R₈, and R₉ can have one or more substituents, and each of the substituents is independently selected from any one of deuterium, an alkyl group with a carbon atom number of 1 to 10, and a cycloalkyl group with a carbon atom number of 3 to 30.

5. The composition according to claim 4, characterized in that Ar₄ is selected from any one of

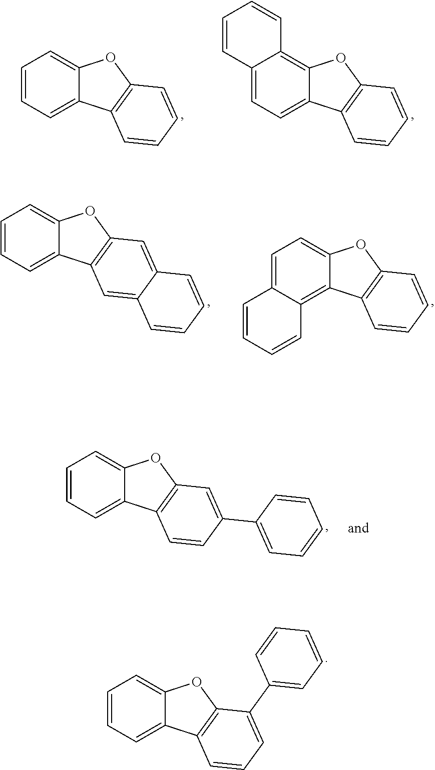

6. The composition according to claim 4, characterized in that the compound represented by Formula II is selected from any one of the following compounds:

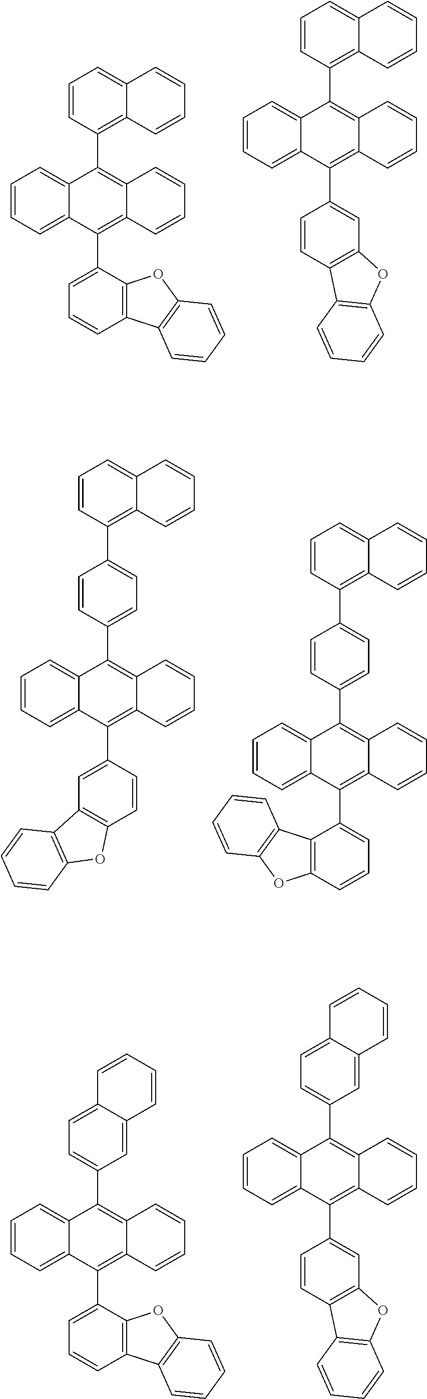 [SMILES: c1ccc2c(-c3c4ccccc4c(-c4cccc5c4oc4ccccc45)c4ccccc34)cccc2c1 ; c1ccc2c(-c3c4ccccc4c(-c4ccc5c(c4)oc4ccccc45)c4ccccc34)cccc2c1 ; c1ccc2c(-c3ccc(-c4c5ccccc5c(-c5ccc6oc7ccccc7c6c5)c5ccccc45)cc3)cccc2c1 ; c1ccc2c(-c3ccc(-c4c5ccccc5c(-c5cccc6oc7ccccc7c56)c5ccccc45)cc3)cccc2c1 ; c1ccc2cc(-c3c4ccccc4c(-c4cccc5c4oc4ccccc45)c4ccccc34)ccc2c1 ; c1ccc2cc(-c3c4ccccc4c(-c4ccc5c(c4)oc4ccccc45)c4ccccc34)ccc2c1]

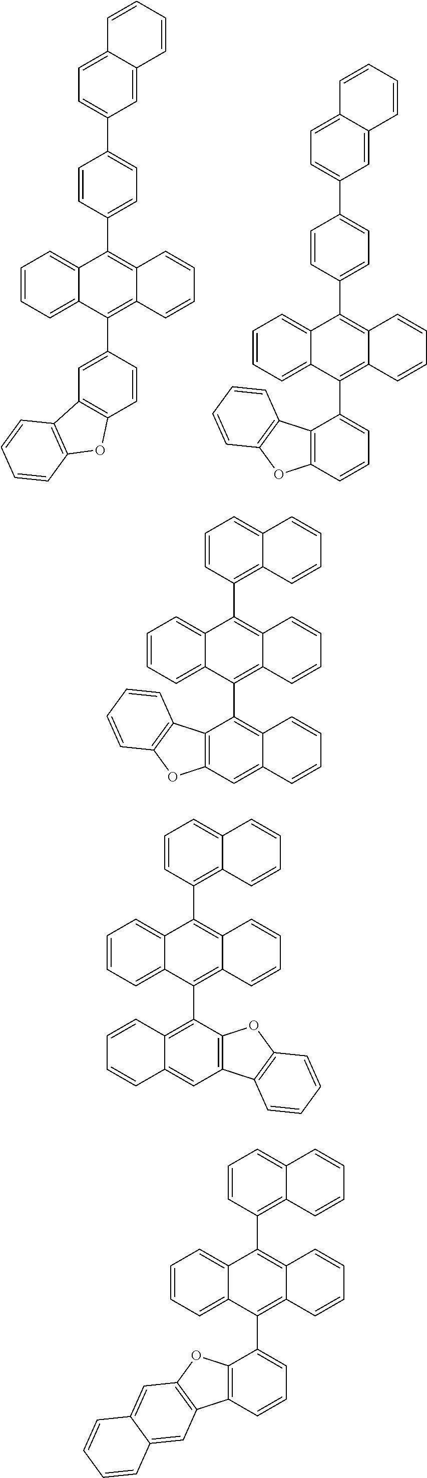

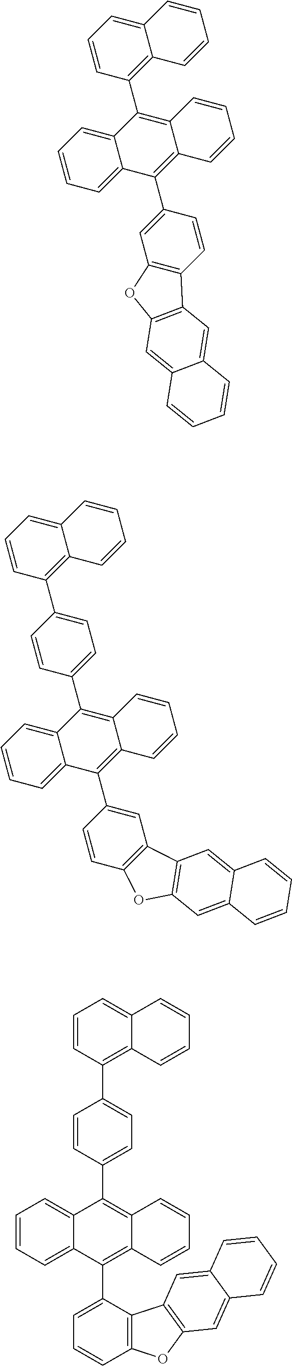 [SMILES: c1ccc2cc3c(cc2c1)oc1cc(-c2c4ccccc4c(-c4cccc5ccccc45)c4ccccc24)ccc13 ; c1ccc2cc3c(cc2c1)oc1ccc(-c2c4ccccc4c(-c4ccc(-c5cccc6ccccc56)cc4)c4ccccc24)cc13 ; c1ccc2cc3c(cc2c1)oc1cccc(-c2c4ccccc4c(-c4ccc(-c5cccc6ccccc56)cc4)c4ccccc24)c13]

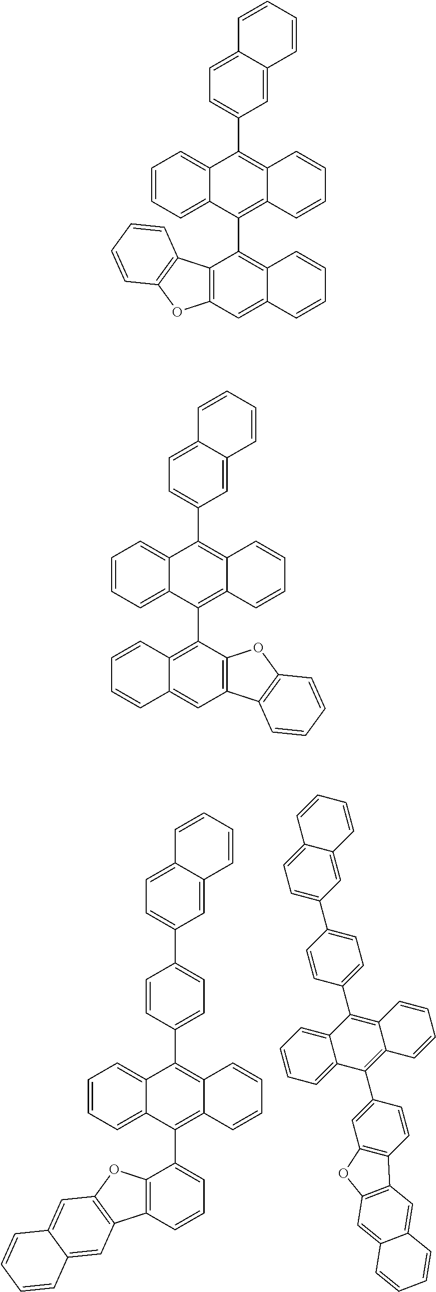

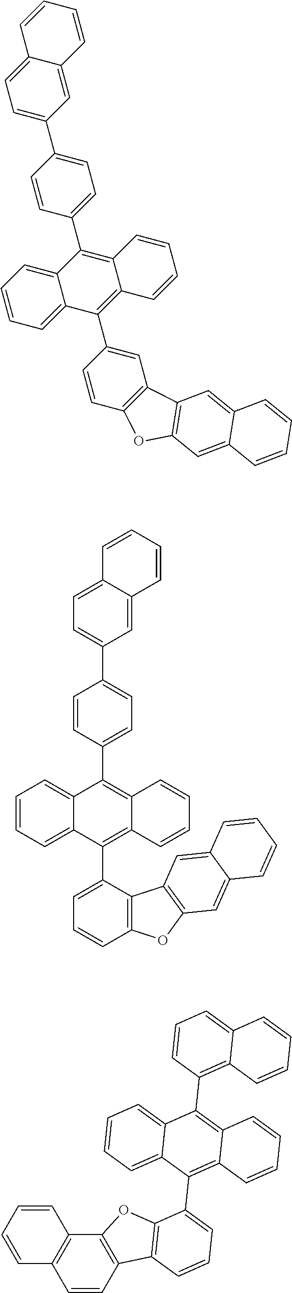 [SMILES: c1ccc2cc(-c3ccc(-c4c5ccccc5c(-c5ccc6oc7cc8ccccc8cc7c6c5)c5ccccc45)cc3)ccc2c1 ; c1ccc2cc(-c3ccc(-c4c5ccccc5c(-c5cccc6oc7cc8ccccc8cc7c56)c5ccccc45)cc3)ccc2c1 ; c1ccc2c(-c3c4ccccc4c(-c4cccc5c4oc4c6ccccc6ccc54)c4ccccc34)cccc2c1]

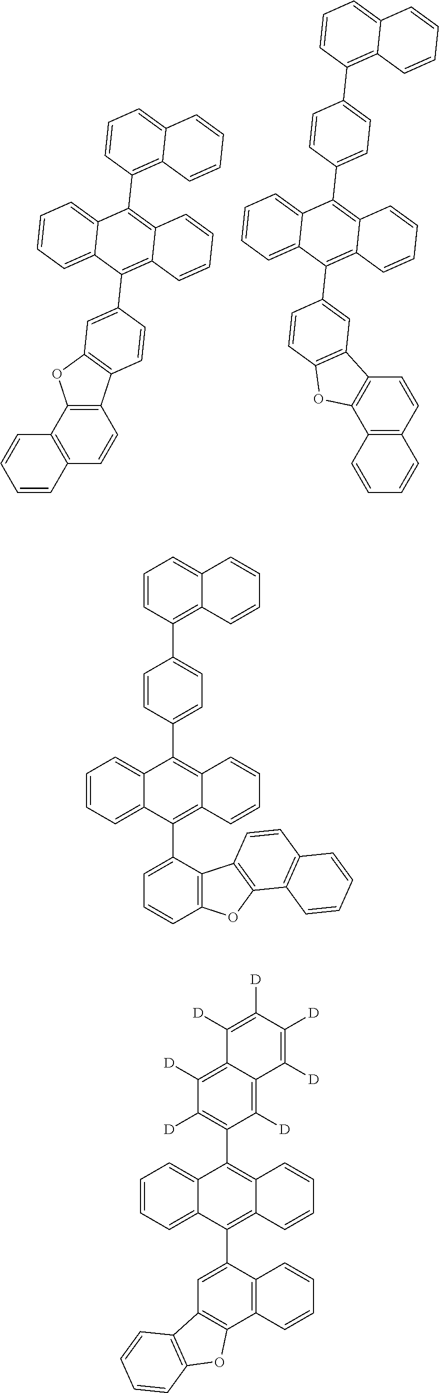

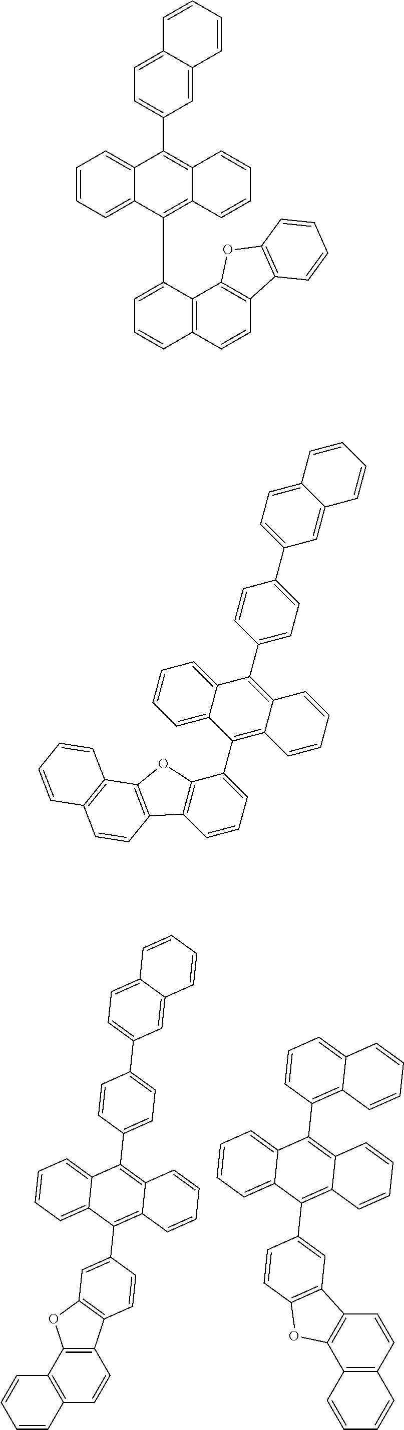

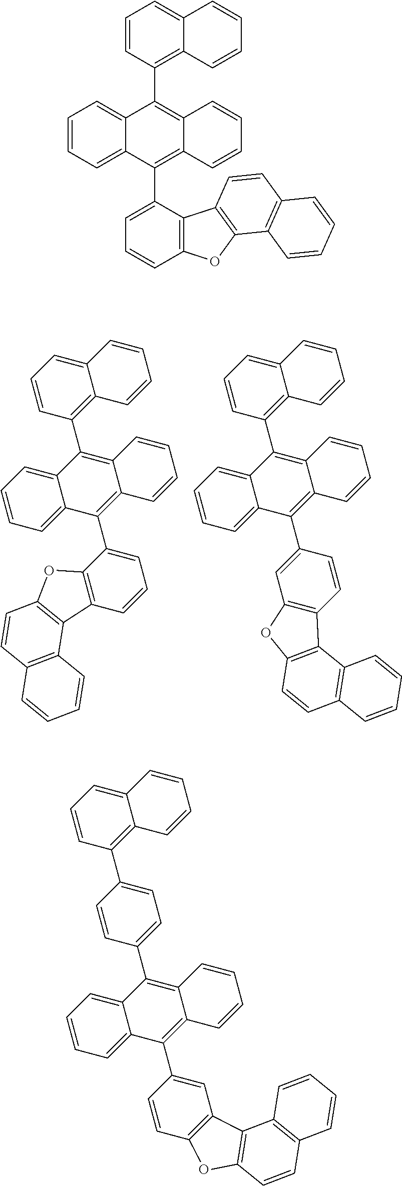

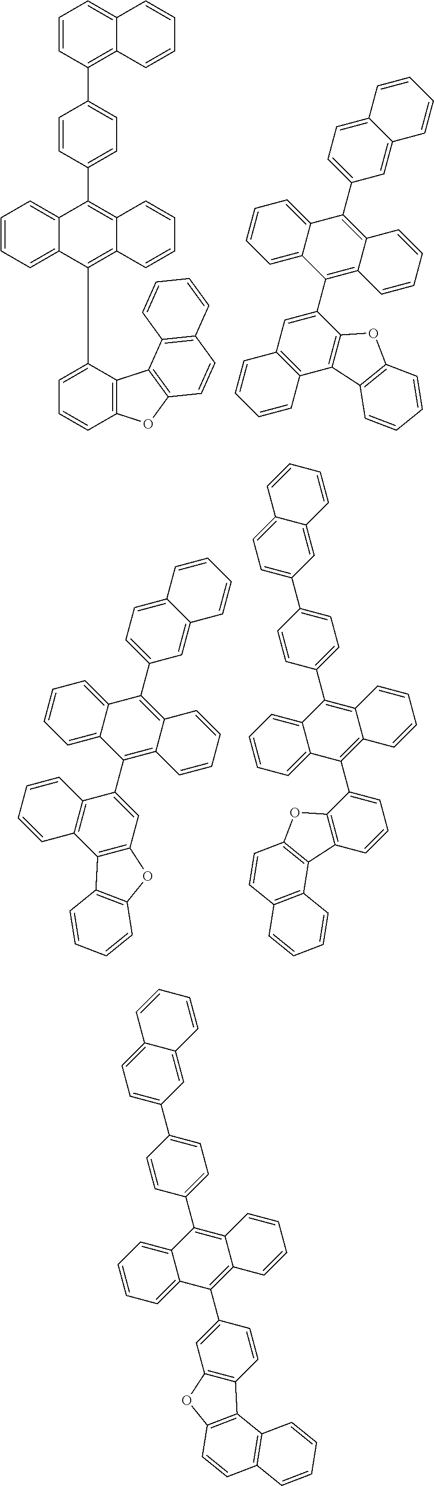

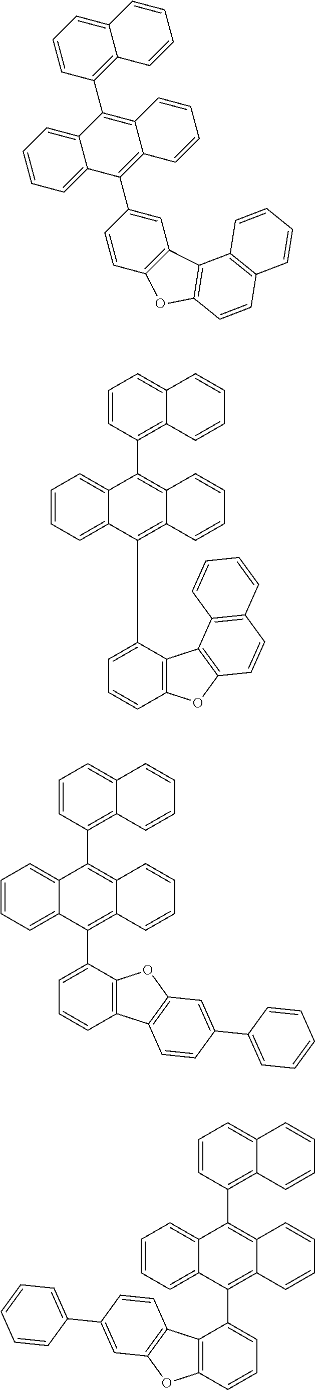 [SMILES: c1ccc2c(-c3c4ccccc4c(-c4ccc5oc6ccc7ccccc7c6c5c4)c4ccccc34)cccc2c1 ; c1ccc2c(-c3c4ccccc4c(-c4cccc5oc6ccc7ccccc7c6c45)c4ccccc34)cccc2c1 ; c1ccc(-c2ccc3c(c2)oc2c(-c4c5ccccc5c(-c5cccc6ccccc56)c5ccccc45)cccc23)cc1 ; c1ccc(-c2ccc3c(c2)oc2cccc(-c4c5ccccc5c(-c5cccc6ccccc56)c5ccccc45)c23)cc1]

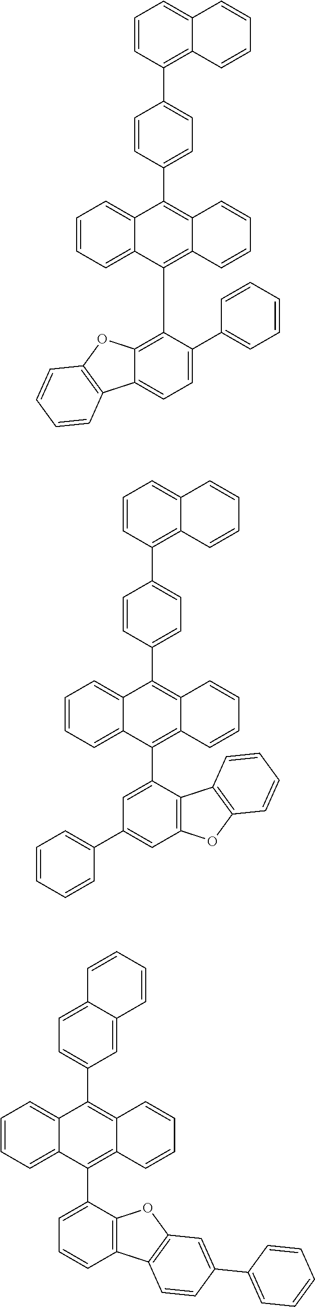 [SMILES: c1ccc(-c2ccc3c(oc4ccccc43)c2-c2c3ccccc3c(-c3ccc(-c4cccc5ccccc45)cc3)c3ccccc23)cc1 ; c1ccc(-c2cc(-c3c4ccccc4c(-c4ccc(-c5cccc6ccccc56)cc4)c4ccccc34)c3c(c2)oc2ccccc23)cc1 ; c1ccc(-c2ccc3c(c2)oc2c(-c4c5ccccc5c(-c5ccc6ccccc6c5)c5ccccc45)cccc23)cc1]

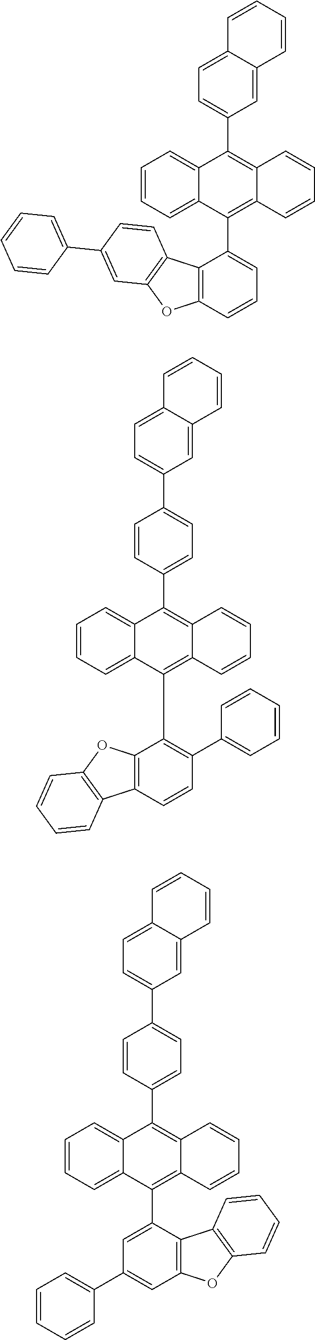 [SMILES: c1ccc(-c2ccc3c(c2)oc2cccc(-c4c5ccccc5c(-c5ccc6ccccc6c5)c5ccccc45)c23)cc1 ; c1ccc(-c2ccc3c(oc4ccccc43)c2-c2c3ccccc3c(-c3ccc(-c4ccc5ccccc5c4)cc3)c3ccccc23)cc1 ; c1ccc(-c2cc(-c3c4ccccc4c(-c4ccc(-c5ccc6ccccc6c5)cc4)c4ccccc34)c3c(c2)oc2ccccc23)cc1]

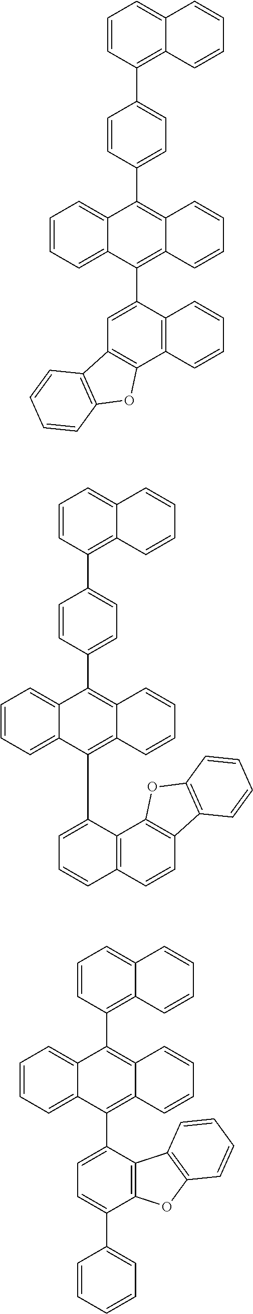 [SMILES: c1ccc2c(-c3ccc(-c4c5ccccc5c(-c5cc6c7ccccc7oc6c6ccccc56)c5ccccc45)cc3)cccc2c1 ; c1ccc2c(-c3ccc(-c4c5ccccc5c(-c5cccc6ccc7c8ccccc8oc7c56)c5ccccc45)cc3)cccc2c1 ; c1ccc(-c2ccc(-c3c4ccccc4c(-c4cccc5ccccc45)c4ccccc34)c3c2oc2ccccc23)cc1]

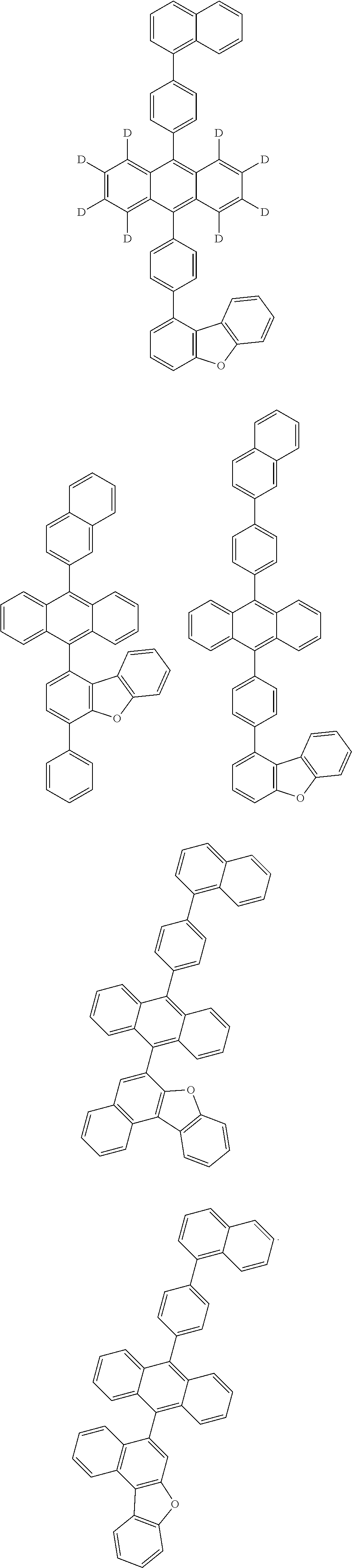

7. The composition according to claim 4, characterized in that the compound represented by Formula III is selected from any one of the following structures represented by Formulas III-1 to III-2:

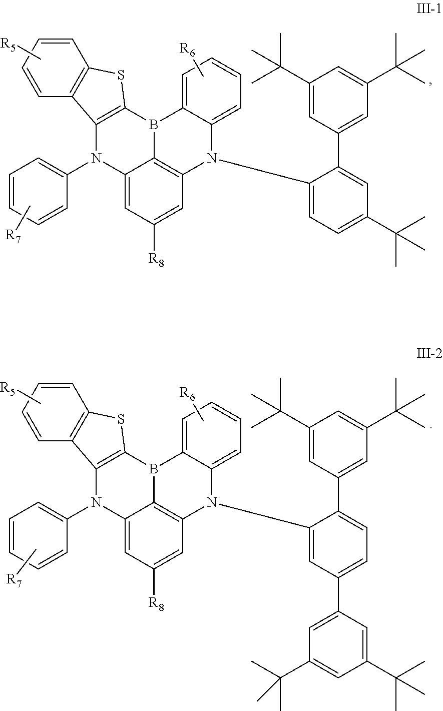

8. The composition according to claim 4, characterized in that the compound represented by Formula III is selected from any one of the following compounds:

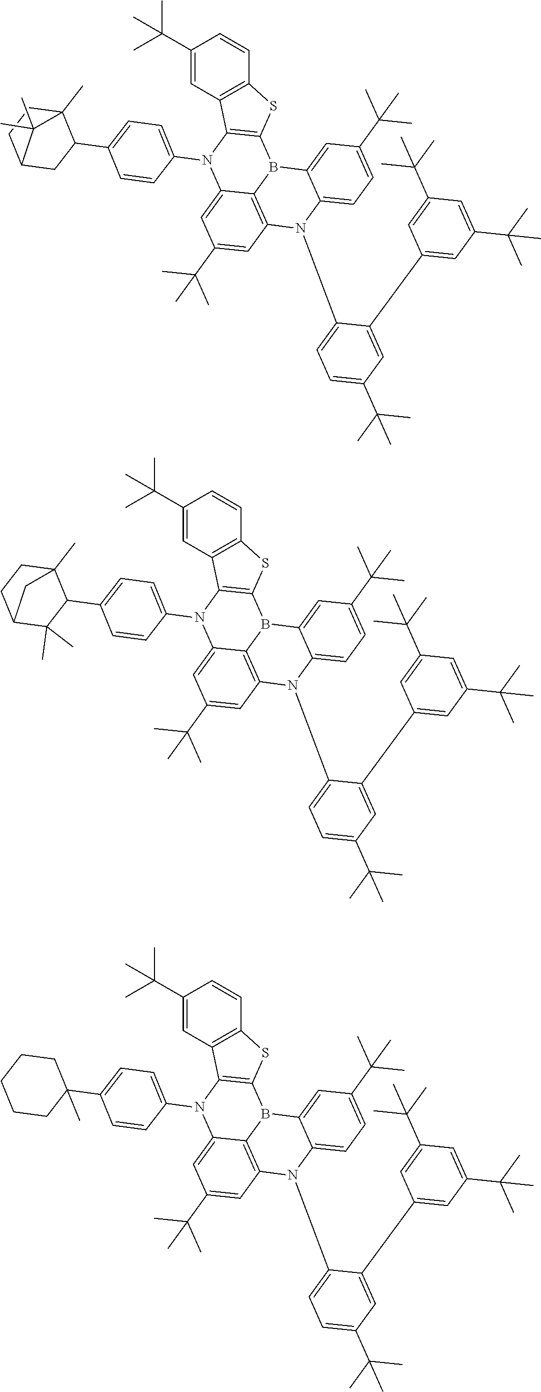 [SMILES: CC(C)(C)c1cc(-c2cc(C(C)(C)C)ccc2N2c3ccc(C(C)(C)C)cc3B3c4sc5ccc(C(C)(C)C)cc5c4N(c4ccc(C5CC6CCC5(C)C6(C)C)cc4)c4cc(C(C)(C)C)cc2c43)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(C5C6(C)CCC(C6)C5(C)C)cc4)c4cc(C(C)(C)C)cc2c43)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(C5(C)CCCCC5)cc4)c4cc(C(C)(C)C)cc2c43)cc(C(C)(C)C)c1]

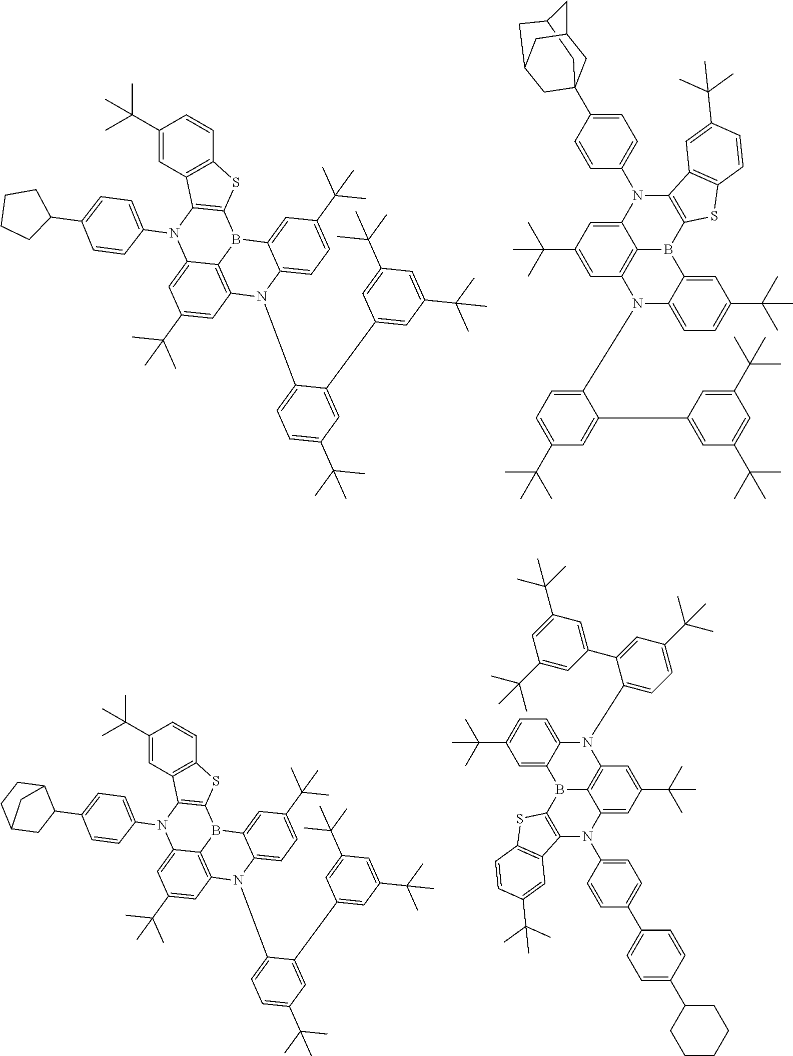

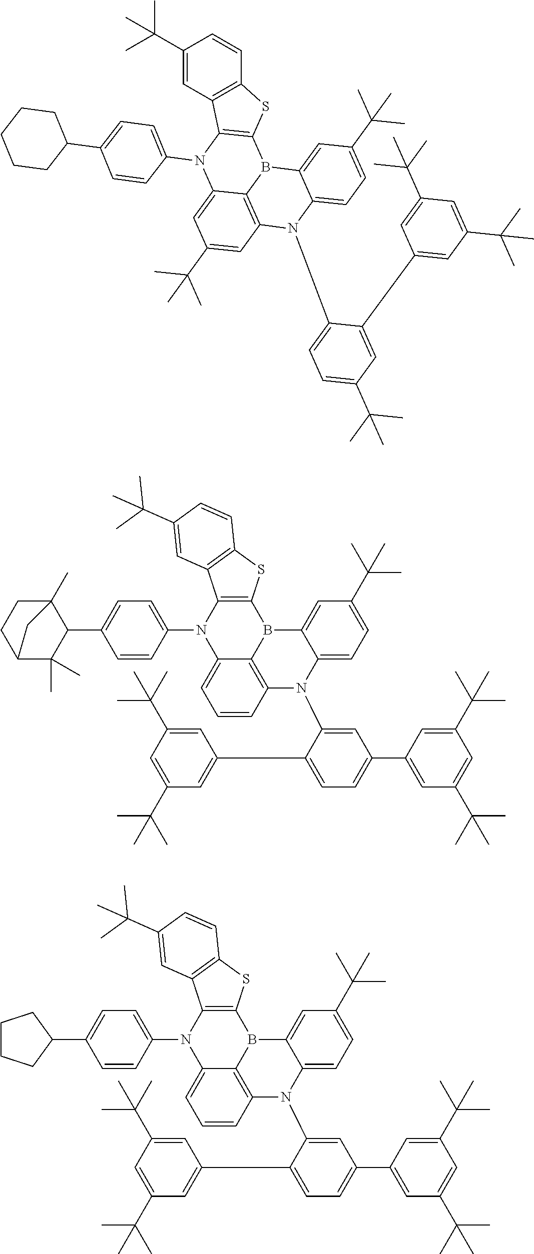

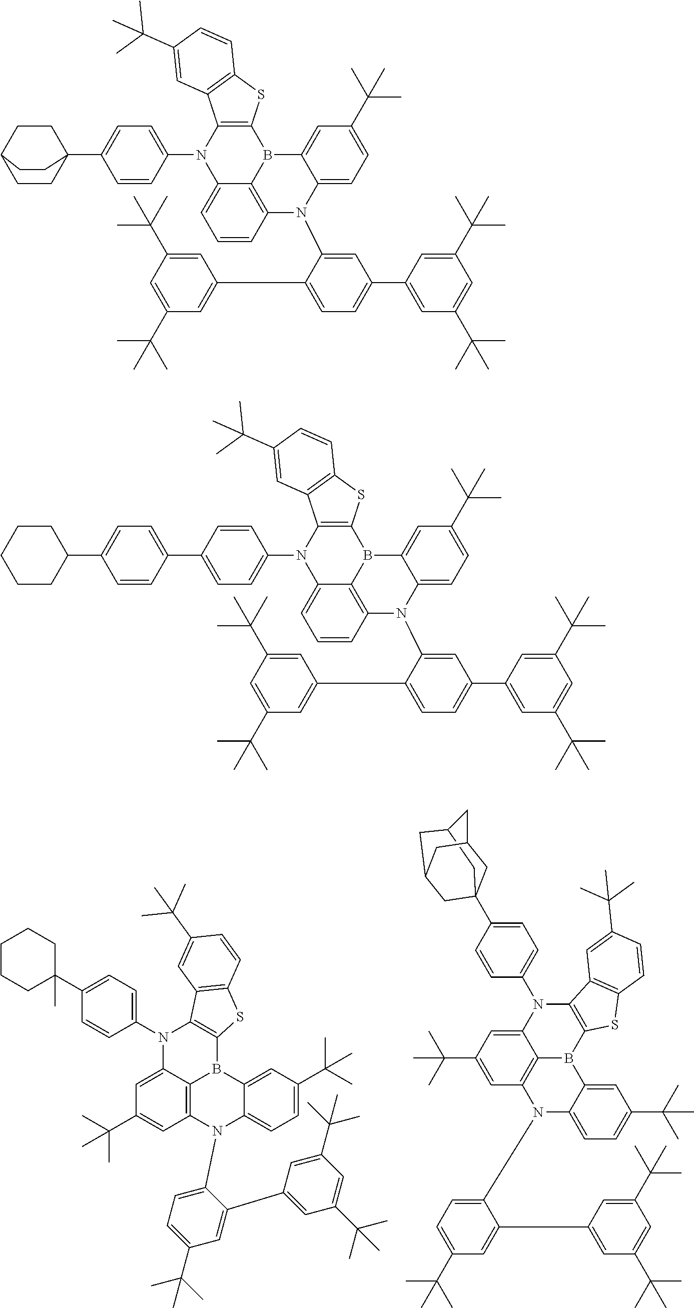

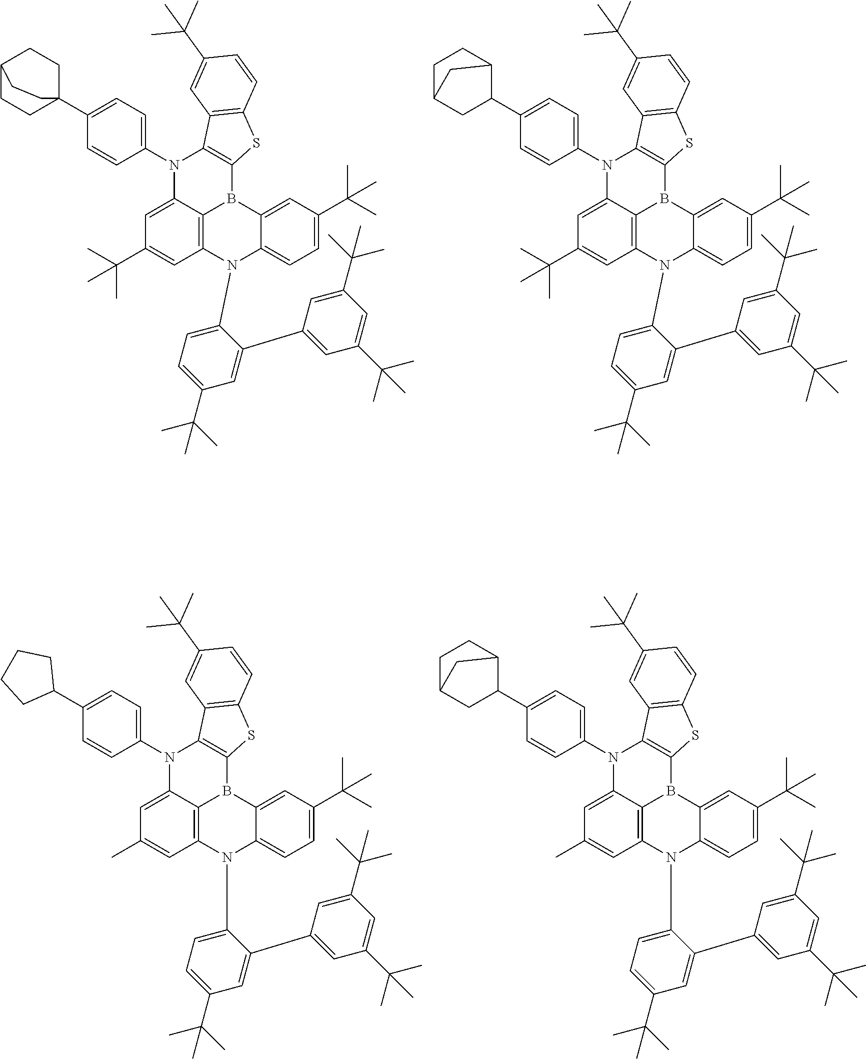

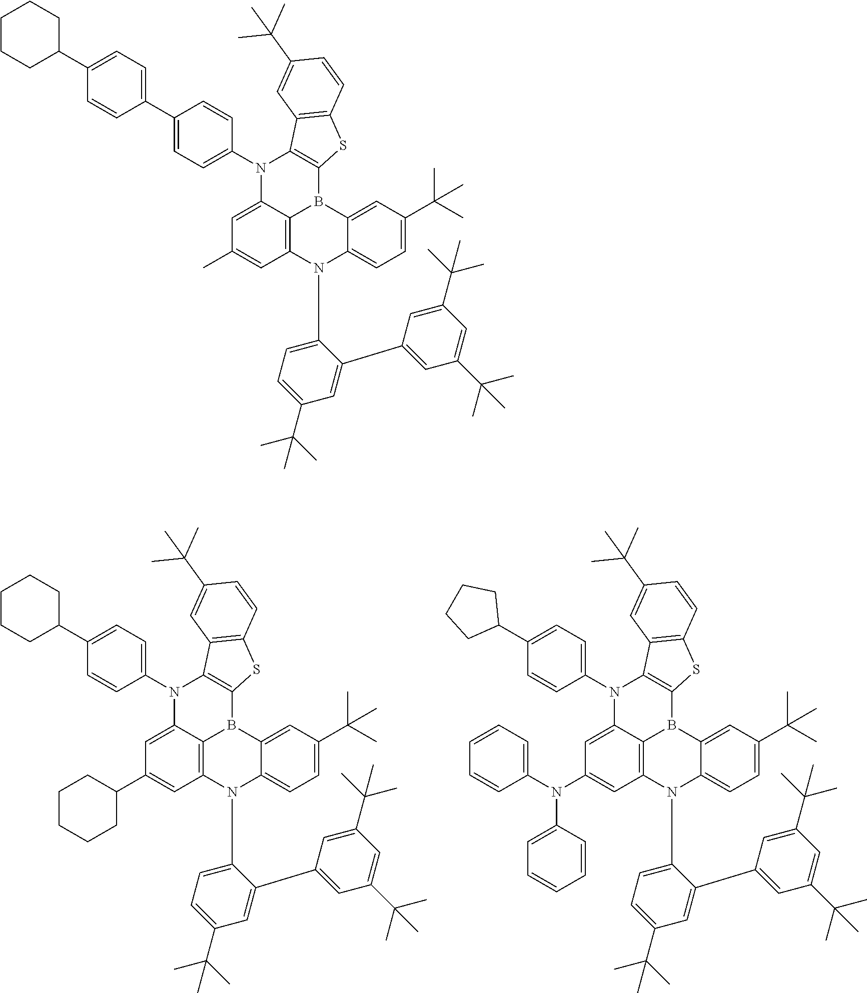 [SMILES: Cc1cc2c3c(c1)N(c1ccc(-c4ccc(C5CCCCC5)cc4)cc1)c1c(sc4ccc(C(C)(C)C)cc14)B3c1cc(C(C)(C)C)ccc1N2c1ccc(C(C)(C)C)cc1-c1cc(C(C)(C)C)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(C5CCCCC5)cc4)c4cc(C5CCCCC5)cc2c43)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(C5CCCC5)cc4)c4cc(N(c5ccccc5)c5ccccc5)cc2c43)cc(C(C)(C)C)c1]

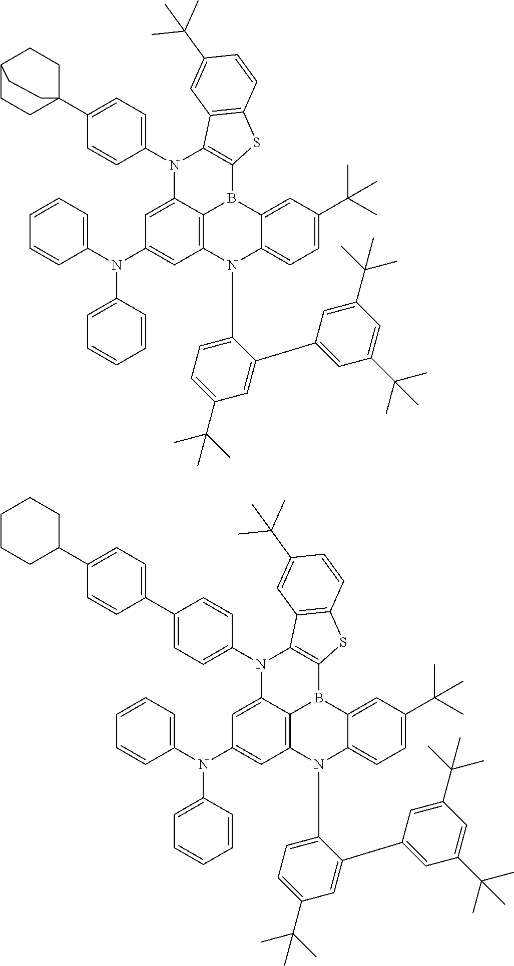 [SMILES: CC(C)(C)c1cc(-c2cc(C(C)(C)C)ccc2N2c3ccc(C(C)(C)C)cc3B3c4sc5ccc(C(C)(C)C)cc5c4N(c4ccc(C56CCC(CC5)CC6)cc4)c4cc(N(c5ccccc5)c5ccccc5)cc2c43)cc(C(C)(C)C)c1 ; CC(C)(C)c1cc(-c2cc(C(C)(C)C)ccc2N2c3ccc(C(C)(C)C)cc3B3c4sc5ccc(C(C)(C)C)cc5c4N(c4ccc(-c5ccc(C6CCCCC6)cc5)cc4)c4cc(N(c5ccccc5)c5ccccc5)cc2c43)cc(C(C)(C)C)c1]

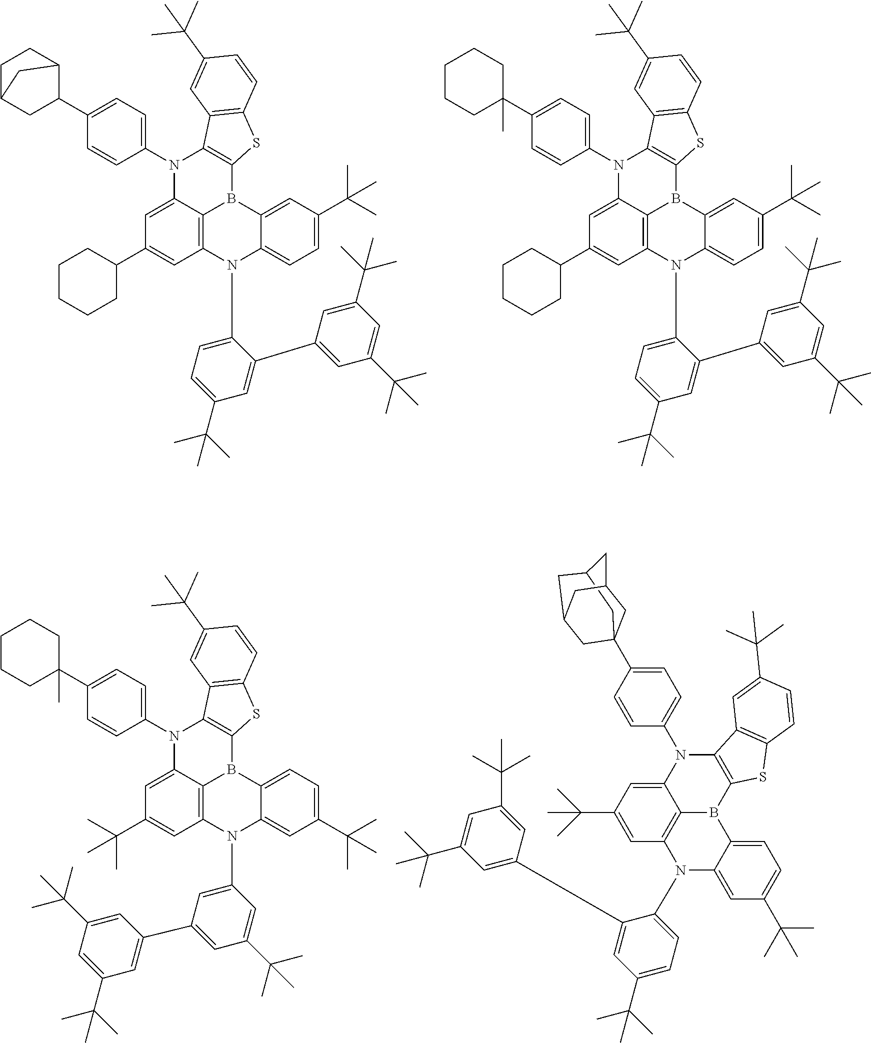

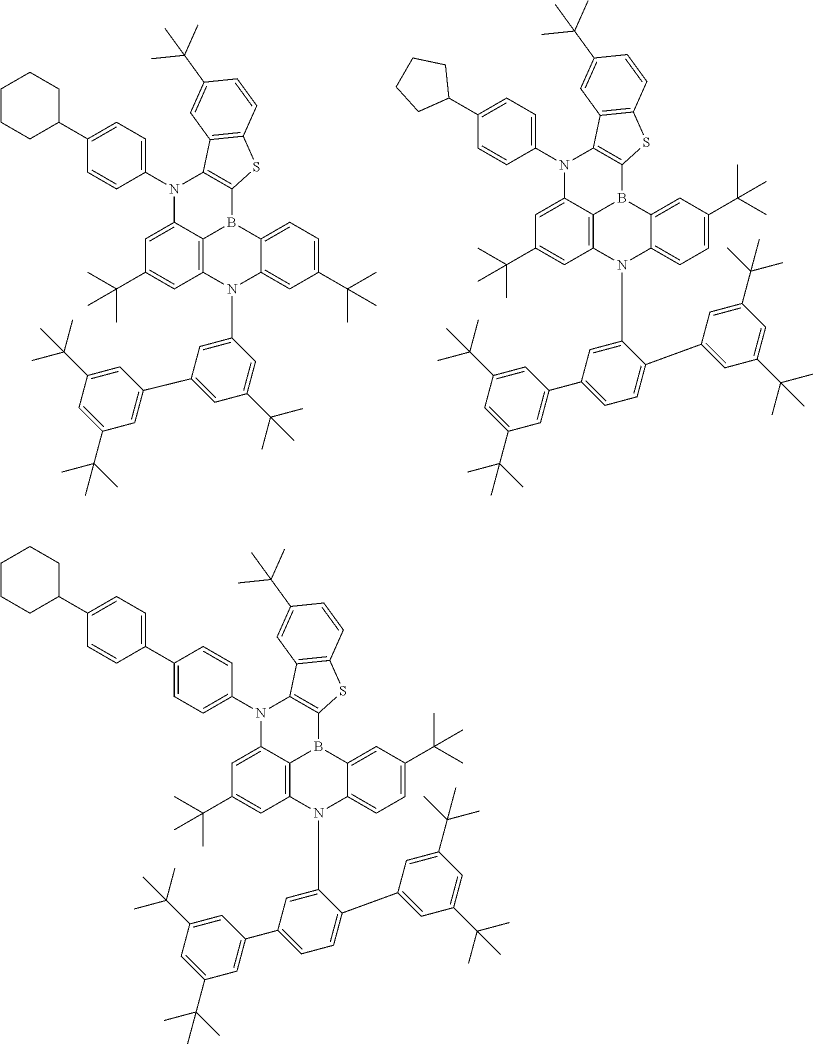 [SMILES: CC(C)(C)c1cc(-c2cc(C(C)(C)C)cc(C(C)(C)C)c2)cc(N2c3cc(C(C)(C)C)ccc3B3c4sc5ccc(C(C)(C)C)cc5c4N(c4ccc(C5CCCCC5)cc4)c4cc(C(C)(C)C)cc2c43)c1 ; CC(C)(C)c1cc(-c2ccc(-c3cc(C(C)(C)C)cc(C(C)(C)C)c3)c(N3c4ccc(C(C)(C)C)cc4B4c5sc6ccc(C(C)(C)C)cc6c5N(c5ccc(C6CCCC6)cc5)c5cc(C(C)(C)C)cc3c54)c2)cc(C(C)(C)C)c1 ; CC(C)(C)c1cc(-c2ccc(-c3cc(C(C)(C)C)cc(C(C)(C)C)c3)c(N3c4ccc(C(C)(C)C)cc4B4c5sc6ccc(C(C)(C)C)cc6c5N(c5ccc(-c6ccc(C7CCCCC7)cc6)cc5)c5cc(C(C)(C)C)cc3c54)c2)cc(C(C)(C)C)c1]

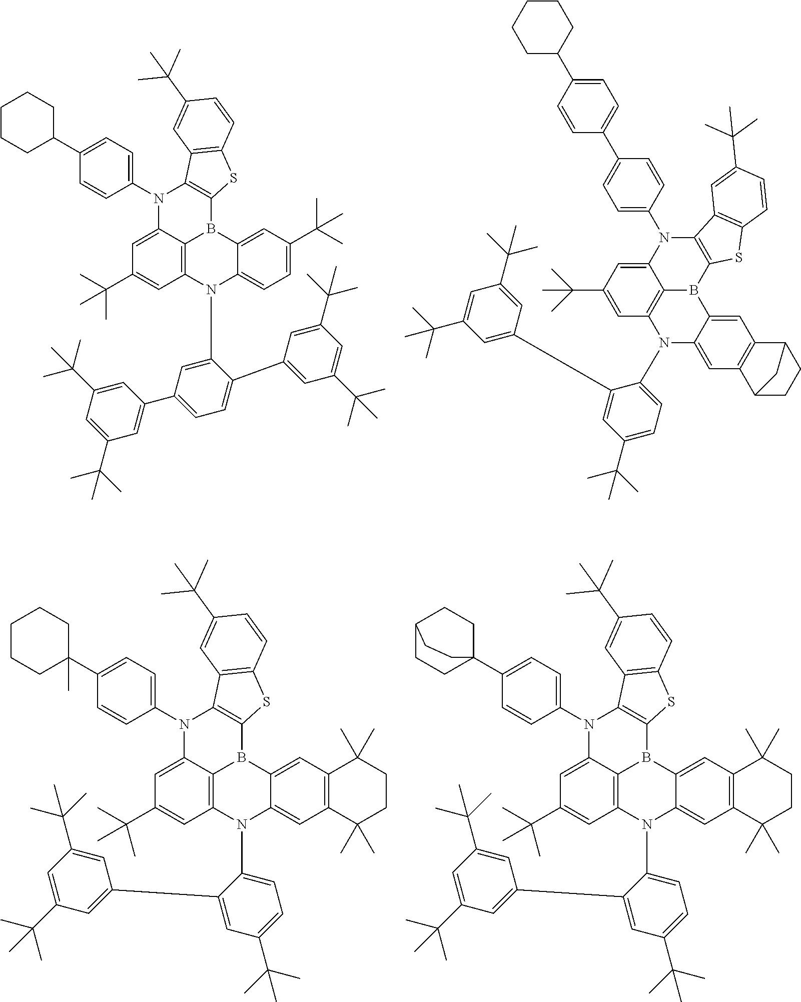

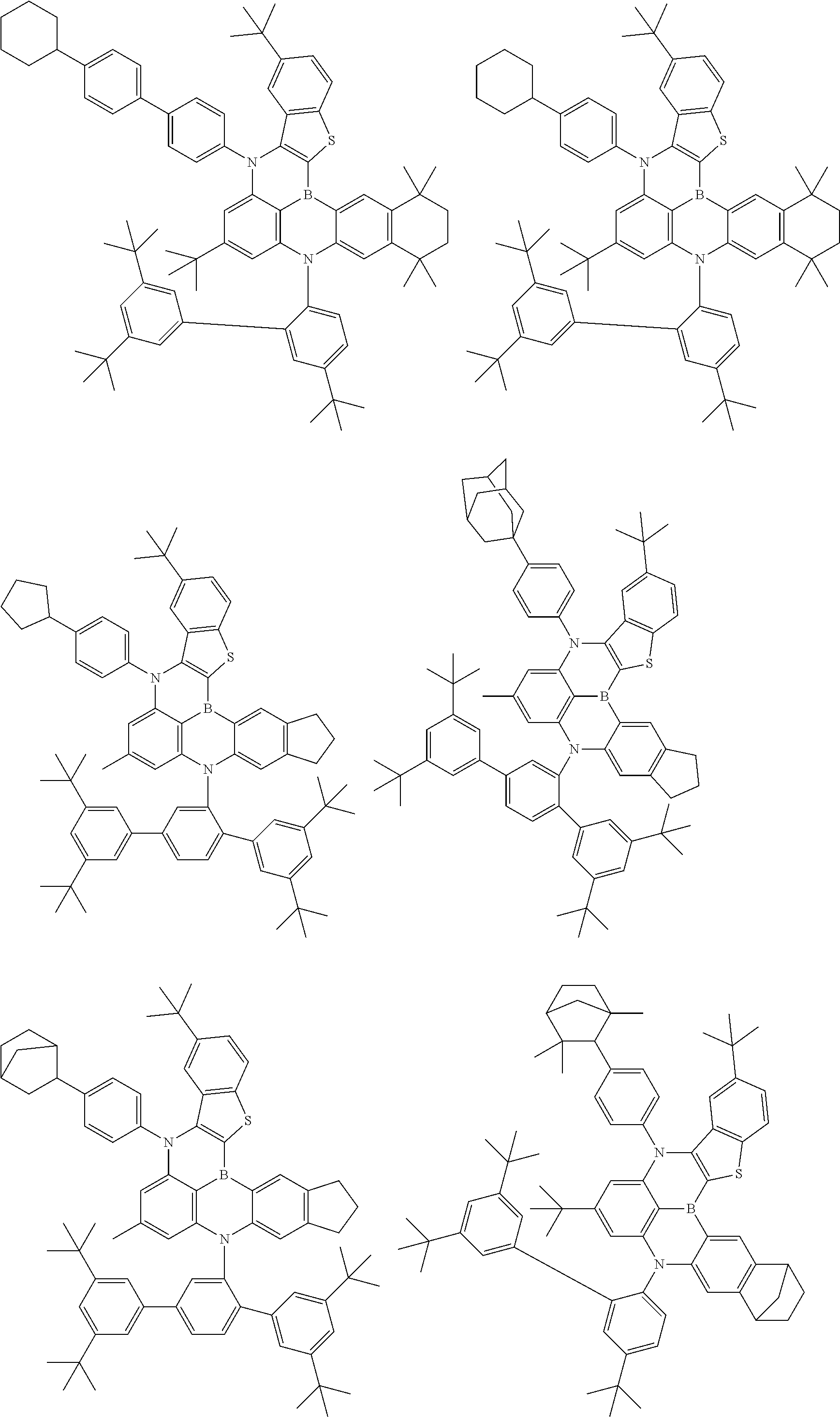

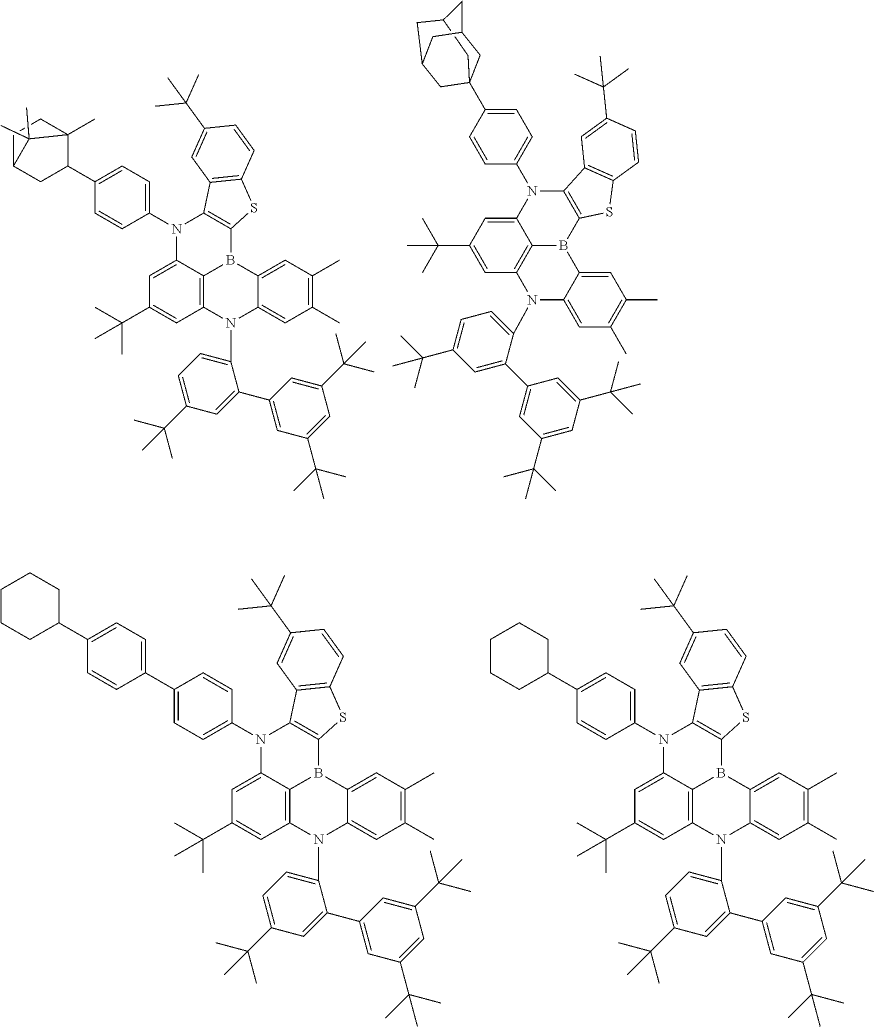

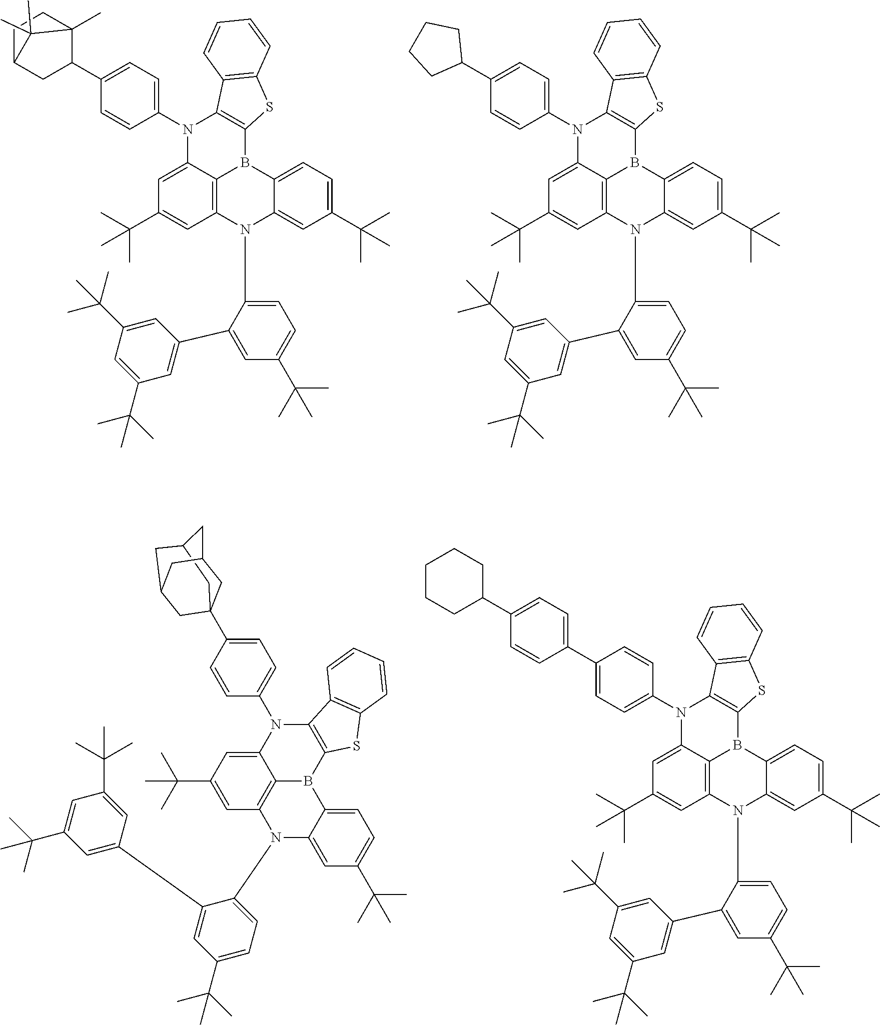

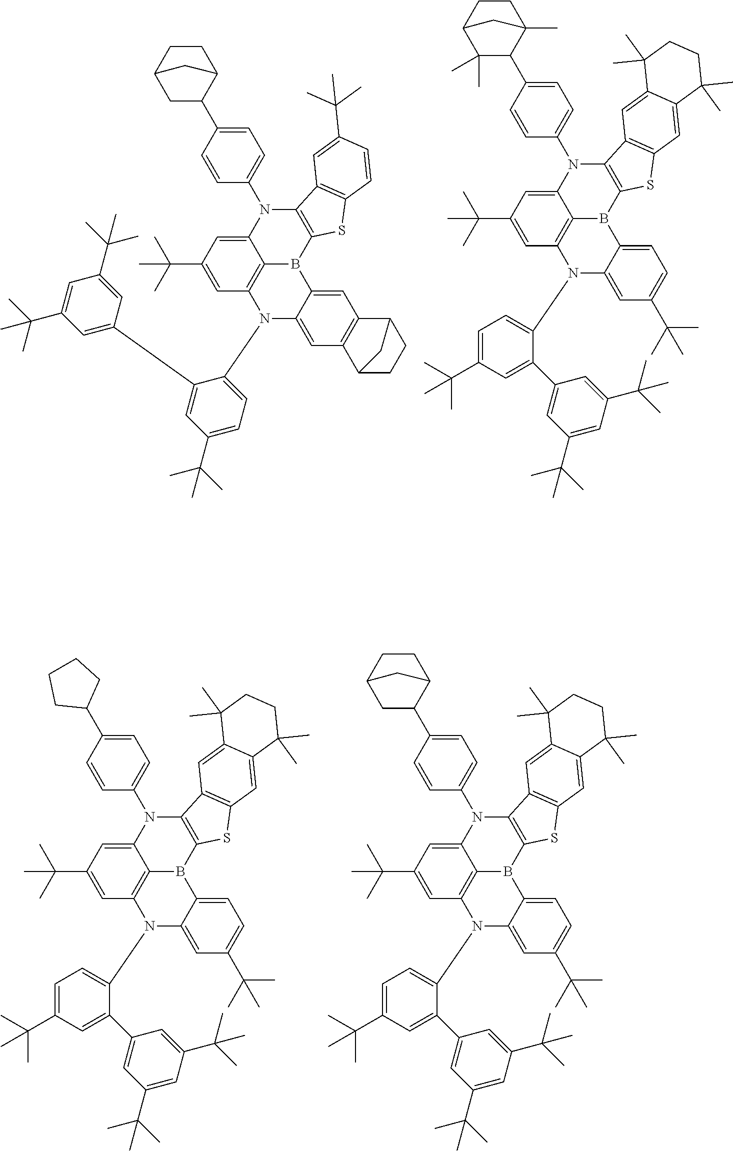

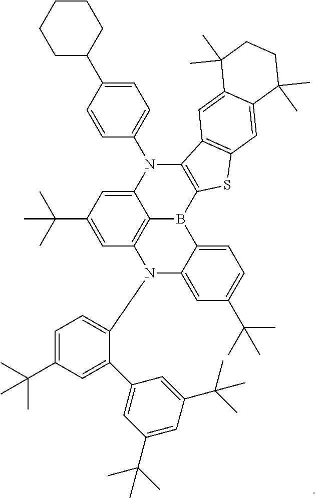 [SMILES: CC(C)(C)c1cc(-c2cc(C(C)(C)C)ccc2N2c3cc(C(C)(C)C)ccc3B3c4sc5cc6c(cc5c4N(c4ccc(C5CCCCC5)cc4)c4cc(C(C)(C)C)cc2c43)C(C)(C)CCC6(C)C)cc(C(C)(C)C)c1]

9. An organic electroluminescent device, characterized by comprising sequentially a first electrode disposed on a substrate plate, a second electrode disposed opposite to the first electrode, and one or more organic functional layers disposed between the first electrode and the second electrode,
wherein the organic functional layer comprises a luminescent layer comprising the composition according to claim 4.

10. The organic electroluminescent device according to claim 9, characterized in that the luminescent layer comprises a host material and a doping material, wherein the host material comprises one or more compounds represented by Chemical Formula I and one or more compounds represented by Chemical Formula II, and the doping material comprises one or more compounds represented by chemical formula III.
